SEQUENCE LISTING (618 sequences)

SEQ ID NO: 1 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 243 aa, synthetic construct
DIKLQQSGAELARPGASVKMSCKTSGYTFTHYTMHWVKQRPGQGLEWIGYINPSRGHTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 3 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWSSNPLTFGAGTKLELK

SEQ ID NO: 4 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIEDTSKVASGVPYRFSGSGSGTSYSLTISSMEAEDAATYYCQQWSSNPLTFGAGTKLELK

SEQ ID NO: 5 - protein, 243 aa, synthetic construct
DIKLQQSGAELARPGASVKMSCKTSGYTFTHYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 6 - protein, 243 aa, synthetic construct
DIKLQQSGAELARPGASVKMSCKTSGYTHTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 7 - protein, 243 aa, synthetic construct
DIKLQQSGAELARPGASVKMSCKTSGYTFTHYTMHWVKQRPGQGLEWIGYINPSRHYTNHNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 8 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCHQWSSNPLTFGAGTKLELK

SEQ ID NO: 9 - protein, 243 aa, synthetic construct
DIKLQQSGAELARPGASVKMSCKTSGYTFTHYTMHWVKQRPGQGLEWIGYINPSRGYTNHNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 10 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQHWSSNPLTFGAGTKLELK

SEQ ID NO: 11 - protein, 243 aa, synthetic construct
DIKLQQSGAELARPGASVKMSCKTSGYTFTHYTMHWVKQRPGQGLEWIGYINPSRGYTNYNQKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 12 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSHPLTFGAGTKLELK

SEQ ID NO: 13 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVHYMNWYQQKSGTSPKRWIYDTSKVASGVPYRFSGSGSGTSYSLTISSMEAEDAATYYCQQWSSNPLTFGAGTKLELK

SEQ ID NO: 14 - protein, 243 aa, synthetic construct
DIKLQQSGAELARPGASVKMSCKTSGYTFTHYTMHWVKQRPGQGLEWIGYINPSRGYTNYHQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 15 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIEDTSKVASGVPYRFSGSGSGTSYSLTISSMEAEDAATYYCQQWSSNPLTFGAGTKLELK

SEQ ID NO: 16 - protein, 243 aa, synthetic construct
DIKLQQSGAELARPGASVKMSCKTSGYTFTRYTMHWVKQRPGQGLEWIGYINPSRGYENYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 17 - protein, 243 aa, synthetic construct
DIKLQQSGAELARPGASVKMSCKTSGYTFTHYTMHWVKQRPGQGLEWIGYINPSRGYTHYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 18 - protein, 243 aa, synthetic construct
DIKLQQSGAELARPGASVKMSCKTSGYTFTHYTMHWVKQRPGQGLEWIGYINPHRGYTNYNHKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 19 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 20 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWSSNPLTFGAGTKLELK

SEQ ID NO: 21 - protein, 243 aa, synthetic construct
DIKLQQSGAELARPGASVKMSCKTSGYTFWRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 22 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 23 - protein, 119 aa, synthetic construct
DIKLQQSGAELARPGASVKMSCKTSGYTFTHYTMHWVKQRPGQGLEWIGYINPSRGHTNYNQKFKDKATLTTDKSSSTAYMQLSSLTSEDSAVYYCARYYDDHYCLDYWGQGTTLTVSS

SEQ ID NO: 24 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 25 - protein, 119 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 26 - protein, 119 aa, synthetic construct
DIKLQQSGAELARPGASVKMSCKTSGYTFTHYTMHWVKQRPGQGLEWIGYINPSRGYTNENQKFKDKATLTTDKSSSTAYMQLSSLTSEDSAVYYCARYYDDHYCLDYWGQGTTLTVSS

SEQ ID NO: 27 - protein, 119 aa, synthetic construct
DIKLQQSGAELARPGASVKMSCKTSGYTHTHYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 28 - protein, 119 aa, synthetic construct
DIKLQQSGAELARPGASVKMSCKTSGYTFTHYTMHWVKQRPGQGLEWIGYINPSRHYTNHNQKFKDKATLTTDKSSSTAYMQLSSLTSEDSAVYYCARYYDDHYCLDYWGQGTTLTVSS

SEQ ID NO: 29 - protein, 119 aa, synthetic construct
DIKLQQSGAELARPGASVKMSCKTSGYTFTHYTMHWVKQRPGQGLEWIGYINPSRGYTNHNQKFKDKATLTTDKSSSTAYMQLSSLTSEDSAVYYCARYYDDHYCLDYWGQGTTLTVSS

SEQ ID NO: 30 - protein, 119 aa, synthetic construct
DIKLQQSGAELARPGASVKMSCKTSGYTFTHYTMHWVKQRPGQGLEWIGYINPSRGYTNYNQKVKDKATLTTDKSSSTAYMQLSSLTSEDSAVYYCARYYDDHYCLDYWGQGTTLTVSS

SEQ ID NO: 31 - protein, 119 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSS

SEQ ID NO: 32 - protein, 119 aa, synthetic construct
DIKLQQSGAELARPGASVKMSCKTSGYTFTHYTMHWVKQRPGQGLEWIGYINPSRGYTNYHQKFKDKATLTTDKSSSTAYMQLSSLTSEDSAVYYCARYYDDHYCLDYWGQGTTLTVSS

SEQ ID NO: 33 - protein, 119 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSS

SEQ ID NO: 34 - protein, 119 aa, synthetic construct
DIKLQQSGAELARPGASVKMSCKTSGYTFTRYTMHWVKQRPGQGLEWIGYINPSRGYENYNQKFKDKATLTTDKSSSTAYMQLSSLTSEDSAVYYCARYYDDHYCLDYWGQGTTLTVSS

SEQ ID NO: 35 - protein, 119 aa, synthetic construct
DIKLQQSGAELARPGASVKMSCKTSGYTFTHYTMHWVKQRPGQGLEWIGYINPSRGYTHYNQKFKDKATLTTDKSSSTAYMQLSSLTSEDSAVYYCARYYDDHYCLDYWGQGTTLTVSS

SEQ ID NO: 36 - protein, 119 aa, synthetic construct
DIKLQQSGAELARPGASVKMSCKTSGYTFTHYTMHWVKQRPGQGLEWIGYINPHRGYTNYNHKFKDKATLTTDKSSSTAYMQLSSLTSEDSAVYYCARYYDDHYCLDYWGQGTTLTVSS

SEQ ID NO: 37 - protein, 119 aa, synthetic construct
DIKLQQSGAELARPGASVKMSCKTSGYTFWRYTMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 38 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 39 - protein, 106 aa, synthetic construct; identical to SEQ ID NO: 38

SEQ ID NO: 40 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQEWSSNPLTFGAGTKLELK

SEQ ID NO: 41 - protein, 106 aa, synthetic construct
DIQLTQSPAIMSASPGEKVTMTCRASSSVSYMNWYQQKSGTSPKRWIEDTSKVASGVPYRFSGSGSGTSYSLTISSMEAEDAATYYCQQWSSNPLTFGAGTKLELK

SEQ ID NO: 42 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCHQWSSNPLTFGAGTKLELK

SEQ ID NO: 43 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQHWSSNPLTFGAGTKLELK

SEQ ID NO: 44 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSHPLTFGAGTKLELK

SEQ ID NO: 45 - protein, 106 aa, synthetic construct
DIQLTQSPAIMSASPGEKVTMTCRASSSVHYMNWYQQKSGTSPKRWIYDTSKVASGVPYRFSGSGSGTSYSLTISSMEAEDAATYYCQQWSSNPLTFGAGTKLELK

SEQ ID NO: 46 - protein, 18 aa, synthetic construct
VEGGSGGSGGSGGSGGVD

SEQ ID NO: 47 - protein, 5 aa, synthetic construct
RYTMH

SEQ ID NO: 48 - protein, 5 aa, synthetic construct
HYTMH

SEQ ID NO: 49 - protein, 17 aa, synthetic construct
YINPSRGYTNYNQKFKD

SEQ ID NO: 50 - protein, 17 aa, synthetic construct
YINPSRGHTNYNQKFKD

SEQ ID NO: 51 - protein, 17 aa, synthetic construct
YINPSRGYTNENQKFKD

SEQ ID NO: 52 - protein, 17 aa, synthetic construct
YINPSRHYTNHNQKFKD

SEQ ID NO: 53 - protein, 17 aa, synthetic construct
YINPSRGYTNHNQKFKD

SEQ ID NO: 54 - protein, 17 aa, synthetic construct
YINPSRGYTNYNQKVKD

SEQ ID NO: 55 - protein, 17 aa, synthetic construct
YINPSRGYTNYHQKFKD

SEQ ID NO: 56 - protein, 17 aa, synthetic construct
YINPSRGYHNYNQKFKD

SEQ ID NO: 57 - protein, 17 aa, synthetic construct
YINPSRGYENYNQKFKD

SEQ ID NO: 58 - protein, 17 aa, synthetic construct
YINPSRGYTHYNQKFKD

SEQ ID NO: 59 - protein, 17 aa, synthetic construct
YINPHRGYTNYNHKFKD

SEQ ID NO: 60 - protein, 10 aa, synthetic construct
YYDDHYCLDY

SEQ ID NO: 61 - protein, 10 aa, synthetic construct
YTDDHYCLDY

SEQ ID NO: 62 - protein, 10 aa, synthetic construct
RASSSVSYMN

SEQ ID NO: 63 - protein, 10 aa, synthetic construct
RASSSVHYMN

SEQ ID NO: 64 - protein, 7 aa, synthetic construct
DTSKVAS

SEQ ID NO: 65 - protein, 9 aa, synthetic construct
QQWSSNPLT

SEQ ID NO: 66 - protein, 9 aa, synthetic construct
QEWSSNPLT

SEQ ID NO: 67 - protein, 9 aa, synthetic construct
HQWSSNPLT

SEQ ID NO: 68 - protein, 9 aa, synthetic construct
QHWSSNPLT

SEQ ID NO: 69 - protein, 9 aa, synthetic construct
QQWSSHPLT

SEQ ID NO: 70 - protein, 5 aa, synthetic construct
  VARIANT 1: note = R or H
XYTMH

SEQ ID NO: 71 - protein, 17 aa, synthetic construct
  VARIANT 1: note = Y or E
  VARIANT 7: note = G or H
  VARIANT 8: note = Y or H
  VARIANT 11: note = Y, E, or H
XINPSRXXTNXNQKFKD

SEQ ID NO: 72 - protein, 10 aa, synthetic construct
  VARIANT 2: note = Y or T
YXDDHYCLDY

SEQ ID NO: 73 - protein, 9 aa, synthetic construct
  VARIANT 2: note = Q or E
QXWSSNPLT

SEQ ID NO: 74 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = T, H, or E
  VARIANT 10: note = N or H
  VARIANT 11: note = Y, E, or H
  VARIANT 12: note = N or H
  VARIANT 13: note = Q or H
  VARIANT 15: note = F or V
XINPXRXXXXXXXKXKD

SEQ ID NO: 75 - protein, 10 aa, synthetic construct
  VARIANT 7: note = S or H
RASSSVXYMN

SEQ ID NO: 76 - protein, 9 aa, synthetic construct
  VARIANT 1: note = Q or H
  VARIANT 2: note = Q or E
  VARIANT 6: note = N or H
XXWSSXPLT

SEQ ID NO: 77 - protein, 17 aa, synthetic construct
  VARIANT 7: note = G or H
  VARIANT 8: note = Y or H
  VARIANT 11: note = Y, E, or H
EINPSRXXTNXNQKFKD

SEQ ID NO: 78 - protein, 17 aa, synthetic construct
  VARIANT 1: note = Y or E
  VARIANT 8: note = Y or H
  VARIANT 11: note = Y, E, or H
XINPSRHXTNXNQKFKD

SEQ ID NO: 79 - protein, 17 aa, synthetic construct
  VARIANT 1: note = Y or E
  VARIANT 7: note = G or H
  VARIANT 11: note = Y, E, or H
XINPSRXHTNXNQKFKD

SEQ ID NO: 80 - protein, 17 aa, synthetic construct
  VARIANT 5: note = S or R
  VARIANT 7: note = G or H
  VARIANT 8: note = Y or H
  VARIANT 9: note = T, H, or E
  VARIANT 10: note = N or H
  VARIANT 11: note = Y, E, or H
  VARIANT 12: note = N or H
  VARIANT 13: note = Q or H
  VARIANT 15: note = F or V
EINPXRXXXXXXXKXKD

SEQ ID NO: 81 - protein, 17 aa, synthetic construct
  VARIANT 1: note = Y or E
  VARIANT 7: note = G or H
  VARIANT 8: note = Y or H
  VARIANT 9: note = T, H, or E
  VARIANT 10: note = N or H
  VARIANT 11: note = Y, E, or H
  VARIANT 12: note = N or H
  VARIANT 13: note = Q or H
  VARIANT 15: note = F or V
XINPRRXXXXXXXKXKD

SEQ ID NO: 82 - protein, 17 aa, synthetic construct
  VARIANT 1: note = Y or E
  VARIANT 5: note = S or R
  VARIANT 8: note = Y or H
  VARIANT 9: note = T, H, or E
  VARIANT 10: note = N or H
  VARIANT 11: note = Y, E, or H
  VARIANT 12: note = N or H
  VARIANT 13: note = Q or H
  VARIANT 15: note = F or V
XINPXRHXXXXXXKXKD

SEQ ID NO: 83 - protein, 17 aa, synthetic construct
  VARIANT 1: note = Y or E
  VARIANT 5: note = S or R
  VARIANT 7: note = G or H
  VARIANT 9: note = T, H, or E
  VARIANT 10: note = N or H
  VARIANT 11: note = Y, E, or H
  VARIANT 12: note = N or H
  VARIANT 13: note = Q or H
  VARIANT 15: note = F or V
XINPXRXHXXXXXKXKD

SEQ ID NO: 84 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = T, H, or E
  VARIANT 11: note = Y, E, or H
  VARIANT 12: note = N or H
  VARIANT 13: note = Q or H
  VARIANT 15: note = F or V
XINPXRXXXHXXXKXKD

SEQ ID NO: 85 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = T, H, or E
  VARIANT 10: note = N or H
  VARIANT 11: note = Y, E, or H
  VARIANT 13: note = Q or H
  VARIANT 15: note = F or V
XINPXRXXXXXHXKXKD

SEQ ID NO: 86 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = T, H, or E
  VARIANT 10: note = N or H
  VARIANT 11: note = Y, E, or H
  VARIANT 12: note = N or H
  VARIANT 15: note = F or V
XINPXRXXXXXXHKXKD

SEQ ID NO: 87 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = T, H, or E
  VARIANT 10: note = N or H
  VARIANT 11: note = Y, E, or H
  VARIANT 12: note = N or H
  VARIANT 13: note = Q or H
XINPXRXXXXXXXKVKD

SEQ ID NO: 88 - protein, 9 aa, synthetic construct
  VARIANT 2: note = Q or E
  VARIANT 6: note = N or H
HXWSSXPLT

SEQ ID NO: 89 - protein, 9 aa, synthetic construct
  VARIANT 1: note = Q or H
  VARIANT 6: note = N or H
XEWSSXPLT

SEQ ID NO: 90 - protein, 9 aa, synthetic construct
  VARIANT 1: note = Q or H
  VARIANT 2: note = Q or E
XXWSSHPLT

SEQ ID NO: 91 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 92 - protein, 50 aa, synthetic construct
  VARIANT 1..50: note = This sequence may encompass 1-10 SGGGG repeating units
SGGGGSGGGGSGGGGSGGGGSGGGGSGGGGSGGGGSGGGGSGGGGSGGGG

SEQ ID NO: 93 - protein, 54 aa, synthetic construct
  VARIANT 5..54: note = This region may encompass 1-10 SGGGG repeating units
GGGGSGGGGSGGGGSGGGGSGGGGSGGGGSGGGGSGGGGSGGGGSGGGGSGGGG

SEQ ID NO: 94 - protein, 32 aa, synthetic construct
  VARIANT 3..32: note = This region may encompass 1-10 SGG repeating units
GGSGGSGGSGGSGGSGGSGGSGGSGGSGGSGG

SEQ ID NO: 95 - protein, 5 aa, synthetic construct
GGGGS

SEQ ID NO: 96 - protein, 243 aa, synthetic construct
DIKLQQSGAELARPGASVKMSCKTSGYTFTRYTMHWVKQRPGQGLEWIGYINPSRGYHNHNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 97 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIEDTSKVASGVPYRFSGSGSGTSYSLTISSMEAEDAATYYCQQWSSNPLTFGAGTKLELK

SEQ ID NO: 98 - protein, 243 aa, synthetic construct
DIKLQQSGAELARPGASVKMSCKTSGYTFTRHTMHWVKQRPGQGLEWIGYINPSRGYTNHNQKFHDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 99 - protein, 243 aa, synthetic construct
DIKLQQSGAELARPGASVKMSCKTSGYTFTRYTMHWVKQRPGQGLEWIGYINPSRGYHHYNQHFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 100 - protein, 243 aa, synthetic construct
DIKLQQSGAELARPGASVKMSCKTSGYTFTRHTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 101 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 102 - protein, 243 aa, synthetic construct
DIKLQQSGAELARPGASVKMSCKTSGYEFTRYTMHWVKQRPGQGLEWIGYINPSRGYTHYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 103 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDEHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 104 - protein, 243 aa, synthetic construct
DIKLQQSGAELARPGASVKMSCKTSGYTFTRYTMHWVKQRPGQGLEWIGYINPHREYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 105 - protein, 243 aa, synthetic construct
DIKLQQSGAELARPGASVKMSCKTSGYTFTRYTMHWVKQRPGQGLEWIGYINPHRGYEHYNQKFKEKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 106 - protein, 243 aa, synthetic construct
DIKLQQSGAELARPGASVKMSCKTSGYGFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 107 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 108 - protein, 243 aa, synthetic construct
DIKLQQSGAELARPGASVKMSCKTSGYGFTRYTMHWVKQRPGQGLEWIGYINPHRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 109 - protein, 243 aa, synthetic construct
DIKLQQSGAELARPGASVKMSCKTSGYTFTRYTMHWVKQRPGQGLEWIGYINPSRGYTNYNQHFHDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 110 - protein, 243 aa, synthetic construct
DIKLQQSGAELARPGASVKMSCKTSGYTFTRYTMHWVKQRPGQGLEWIGYINPSRGYENYNHKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 111 - protein, 243 aa, synthetic construct
DIKLQQSGAELARPGASVKMSCKTSGYTFTRYTMHWVKQRPGQGLEWIGYINPSRGYENYHQKFKHKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 112 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSHSYMNWYQQKSGTSPKRWIYDTSKVASGVPYRFSGSGSGTSYSLTISSMEAEDAATYYCQQWSSNPLTFGAGTKLELK

SEQ ID NO: 113 - protein, 243 aa, synthetic construct
DIKLQQSGAELARPGASVKMSCKTSGYTFTRYTMHWVKQRPGQGLEWIGYINPSRGYTNYNQKFHDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 114 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDEYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 115 - protein, 243 aa, synthetic construct
DIKLQQSGAELARPGASVKMSCKTSGYHFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 116 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVHYMNWYQQKSGTSPKRWIYDTSKVASGVPYRFSGSGSGTSYSLTISSMEAEDAATYYCQQWSSNPLTFGAGTKLELK

SEQ ID NO: 117 - protein, 243 aa, synthetic construct
DIKLQQSGAELARPGASVKMSCKTSGYTFTRYTMHWVKQRPGQGLEWIGYINPSREYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 118 - protein, 243 aa, synthetic construct
DIKLQQSGAELARPGASVKMSCKTSGYTFTRYTMHWVKQRPGQGLEWIGYINPSRGYTHENQHFKEKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 119 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQHWSSNPLTFGAGTKLELK

SEQ ID NO: 120 - protein, 243 aa, synthetic construct
DIKLQQSGAELARPGASVKMSCKTSGYGFTRYTMHWVKQRPGQGLEWIGYINPSRGYTNYNQKFKVKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 121 - protein, 243 aa, synthetic construct
DIKLQQSGAELARPGASVKMSCKTSGYTFWRYTMHWVKQRPGQGLEWIGYINPSRHYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 122 - protein, 243 aa, synthetic construct
DIKLQQSGAELARPGASVKMSCKTSGYTFTRYTMHWVKQRPGQGLEWIGYHNPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 123 - protein, 243 aa, synthetic construct
DIKLQQSGAELARPGASVKMSCKTSGHTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 124 - protein, 243 aa, synthetic construct
DIKLQQSGAELARPGASVKMSCKTSGYTFTRYTQHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 125 - protein, 243 aa, synthetic construct
DIKLQQSGAELARPGASVKMSCKTSGYTFTRYTMHWVKQRPGQGLEWIGYINPSRGYTHYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 126 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYEDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 127 - protein, 243 aa, synthetic construct
DIKLQQSGAELARPGASVKMSCKTSGYTFTRYTMHWVKQRPGQGLEWIGYINPSRGYTNYHQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 128 - protein, 243 aa, synthetic construct
DIKLQQSGAELARPGASVKMSCKTSGYTFTRYTMHWVKQRPGQGLEWIGYINPSRNYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 129 - protein, 243 aa, synthetic construct
DIKLQQSGAELARPGASVKMSCKTSGYTHTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 130 - protein, 243 aa, synthetic construct
DIKLQQSGAELARPGASVKMSCKTSGYTFTRYTMHWVKQRPGQGLEWIGYINPSRGYTNYNQHFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 131 - protein, 243 aa, synthetic construct
DIKLQQSGAELARPGASVKMSCKTSGYTFTRYTMHWVKQRPGQGLEWIGYINPSRGYTNYNEKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 132 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDHHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 133 - protein, 243 aa, synthetic construct
DIKLQQSGAELARPGASVKMSCKTSGYTFTRYTMHWVKQRPGQGLEWIGYINPSRGYTNHNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 134 - protein, 243 aa, synthetic construct
DIKLQQSGAELARPGASVKMSCKTSGYGFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 135 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 136 - protein, 243 aa, synthetic construct
DIKLQQSGAELARPGASVKMSCKTSGYTFWRYTMHWVKQRPGQGLEWIGYINPSRGYTNYNQKFEDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 137 - protein, 243 aa, synthetic construct
DIKLQQSGAELARPGASVKMSCKTSGYTFTRYTMHWVKQRPGQGLEWIGYINPSRGYTNYNQKFEDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 138 - protein, 243 aa, synthetic construct
DIKLQQSGAELARPGASVKMSCKTSGYTHTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 139 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYHCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 140 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDHHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 141 - protein, 243 aa, synthetic construct
DIKLQQSGAELARPGASVKMSCKTSGYTFTRYTMHWVKQRPGQGLEWIGYINPSRGYHNYNQKFKVKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 142 - protein, 243 aa, synthetic construct
DIKLQQSGAELARPGASVKMSCKTSGYTFHRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 143 - protein, 243 aa, synthetic construct
DIKLQQSGAELARPGASVKMSCKTSGYTFTRYTMHWVKQRPGQGLEWIGYINPSRGYTNYNQKHKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 144 - protein, 243 aa, synthetic construct
DIKLQQSGAELARPGASVKMSCKTSGYTFTRYTHHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 145 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDEHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 146 - protein, 243 aa, synthetic construct
DIKLQQSGAELARPGASVKMSCKTSGYTFTRYTMHWVKQRPGQGLEWIGYINPSRGYTNYNQEFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 147 - protein, 243 aa, synthetic construct
DIKLQQSGAELARPGASVKMSCKTSGYTFTRYTMHWVKQRPGQGLEWIGYINPSRHYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 148 - protein, 243 aa, synthetic construct
DIKLQQSGAELARPGASVKMSCKTSGYTFWRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 149 - protein, 243 aa, synthetic construct
DIKLQQSGAELARPGASVKMSCKTSGYTFTRYTMHWVKQRPGQGLEWIGYINPSRGYHNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 150 - protein, 243 aa, synthetic construct
DIKLQQSGAELARPGASVKMSCKTSGYGFTRYTMHWVKQRPGQGLEWIGYINPSRNYTNYNQEFKHKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 151 - protein, 243 aa, synthetic construct
DIKLQQSGAELARPGASVKMSCKTSGYTFTRYTMHWVKQRPGQGLEWIGYINPSRHYTNYNQKHKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 152 - protein, 243 aa, synthetic construct
DIKLQQSGAELARPGASVKMSCKTSGYTHTRYTMHWVKQRPGQGLEWIGYINPSRGYTNYNEKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 153 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYSARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 154 - protein, 243 aa, synthetic construct
DIKLQQSGAELARPGASVKMSCKTSGYTFTRYTMHWVKQRPGQGLEWIGYINPHRGYTNYHQKHKDKATLTTDKSSSTAYMQLSSLTSEDSAVYYCARYYDDEYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 155 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDEYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 156 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSENPLTFGAGTKLELK

SEQ ID NO: 157 - protein, 243 aa, synthetic construct
DIKLQQSGAELARPGASVKMSCKTSGYTFTRYTMHWVKQRPGQGLEWIGYINPSRGYTNYNHKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 158 - protein, 243 aa, synthetic construct
DIKLQQSGAELARPGASVKMSCKTSGYTFTRYTMHWVKQRPGQGLEWIGYENPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 159 - protein, 243 aa, synthetic construct
DIKLQQSGAELARPGASVKMSCKTSGYTFTRYTMHWVKQRPGQGLEWIGYINPSRGYTNYNHKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 160 - protein, 243 aa, synthetic construct
DIKLQQSGAELARPGASVKMSCKTSGYTFTRYTMHWVKQRPGQGLEWIGYINPSRGYTHYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 161 - protein, 243 aa, synthetic construct
DIKLQQSGAELARPGASVKMSCKTSGYTFTRYTMHWVKQRPGQGLEWIGYINPSRGYTNYEQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 162 - protein, 243 aa, synthetic construct
DIKLQQSGAELARPGASVKMSCKTSGYTFTRYTMHWVKQRPGQGLEWIGYINPSRGYTNYNQKFKEKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 163 - protein, 243 aa, synthetic construct
DIKLQQSGAELARPGASVKMSCKTSGYGFTRYTMHWVKQRPGQGLEWIGYINPSRGYTNYNQKFKDKATLTTDKSSSTAYMQLSSLTSEDSAVYYCARYHDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 164 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYHDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 165 - protein, 243 aa, synthetic construct
DIKLQQSGAELARPGASVKMSCKTSGYTFTRYTMHWVKQRPGQGLEWIGYINPSRGYTNENQEFKDKATLTTDKSSSTAYMQLSSLTSEDSAVYYCARYYDEHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 166 - protein, 243 aa, synthetic construct
DIKLQQSGAELARPGASVKMSCKTSGYTFTRYTMHWVKQRPGQGLEWIGYINPSRGYTNYNQKFKHKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 167 - protein, 243 aa, synthetic construct
DIKLQQSGAELARPGASVKMSCKTSGYEFTRHTMHWVKQRPGQGLEWIGYINPSRGYTNYNHKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 168 - protein, 243 aa, synthetic construct
DIKLQQSGAELARPGASVKMSCKTSGYTFTRYTMHWVKQRPGQGLEWIGYINPSRGYTNYNQKFKVKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 169 - protein, 243 aa, synthetic construct
DIKLQQSGAELARPGASVKMSCKTSGYTFTRYTMHWVKQRPGQGLEWIGYINPHRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 170 - protein, 243 aa, synthetic construct
DIKLQQSGAELARPGASVKMSCKTSGYTFTRDTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 171 - protein, 243 aa, synthetic construct
DIKLQQSGAELARPGASVKMSCKTSGYEFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 172 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDHHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 173 - protein, 243 aa, synthetic construct
DIKLQQSGAELARPGASVKMSCKTSGYTFTRYTMHWVKQRPGQGLEWIGYINPHRGYTNYNQKFKHKATLTTDKSSSTAYMQLSSLTSEDSAVYHCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 174 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSHSYMNWYQQKSGTSPKRWIYDTSKVASGVPYRFSGSGSGTSYSLTISSMEAEDAATYYCQQWSSNPLTFGAGTKLELK

SEQ ID NO: 175 - protein, 243 aa, synthetic construct
DIKLQQSGAELARPGASVKMSCKTSGYTFTRYTMHWVKQRPGQGLEWIGYINPSRGYTNYNQKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 176 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYHNWYQQKSGTSPKRWIYDTSKVASGVPYRFSGSGSGTSYSLTISSMEAEDAATYYCQQWSSNPLTFGAGTKLELK

SEQ ID NO: 177 - protein, 243 aa, synthetic construct
DIKLQQSGAELARPGASVKMSCKTSGYGFTRYTMHWVKQRPGQGLEWIGYINPSRGYTNYEHKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 178 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDHHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 179 - protein, 243 aa, synthetic construct
DIKLQQSGAELARPGASVKMSCKTSGYTFTRYHMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 180 - protein, 243 aa, synthetic construct
DIKLQQSGAELARPGASVKMSCKTSGYTFTRYTMHHVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 181 - protein, 243 aa, synthetic construct
DIKLQQSGAELARPGASVKMSCKTSGYTFTRYTMHWVKQRPGQGLEWHGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 182 - protein, 243 aa, synthetic construct
DIKLQQSGAELARPGASVKMSCKTSGYTFTRYTMHWVKQRPGQGLEWIHYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 183 - protein, 243 aa, synthetic construct
DIKLQQSGAELARPGASVKMSCKTSGYTFTRYTMHWVKQRPGQGLEWIGHINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 184 - protein, 243 aa, synthetic construct
DIKLQQSGAELARPGASVKMSCKTSGYTFTRYTMHWVKQRPGQGLEWIGYIHPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 185 - protein, 243 aa, synthetic construct
DIKLQQSGAELARPGASVKMSCKTSGYTFTRYTMHWVKQRPGQGLEWIGYINHSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 186 - protein, 243 aa, synthetic construct
DIKLQQSGAELARPGASVKMSCKTSGYTFTRYTMHWVKQRPGQGLEWIGYINPSHGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 187 - protein, 243 aa, synthetic construct
DIKLQQSGAELARPGASVKMSCKTSGYTFTRYTMHWVKQRPGQGLEWIGYINPSRGHTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 188 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVHYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 189 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYHARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 190 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCHRYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 191 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCAHYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 192 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARHYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 193 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYHDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 194 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHHCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 195 - protein, 243 aa, synthetic construct
DIKLQQSGAELARPGASVKMSCKTSGDTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 196 - protein, 243 aa, synthetic construct
DIKLQQSGAELARPGASVKMSCKTSGYTDTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 197 - protein, 243 aa, synthetic construct
DIKLQQSGAELARPGASVKMSCKTSGYTFERYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 198 - protein, 243 aa, synthetic construct
DIKLQQSGAELARPGASVKMSCKTSGYTFTDYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 199 - protein, 243 aa, synthetic construct
DIKLQQSGAELARPGASVKMSCKTSGYTFTRYEMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 200 - protein, 243 aa, synthetic construct
DIKLQQSGAELARPGASVKMSCKTSGYTFTRYTEHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 201 - protein, 243 aa, synthetic construct
DIKLQQSGAELARPGASVKMSCKTSGYTFTRYTMDWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 202 - protein, 243 aa, synthetic construct
DIKLQQSGAELARPGASVKMSCKTSGYTFTRYTMHEVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 203 - protein, 243 aa, synthetic construct
DIKLQQSGAELARPGASVKMSCKTSGYTFTRYTMHWVKQRPGQGLEWDGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 204 - protein, 243 aa, synthetic construct
DIKLQQSGAELARPGASVKMSCKTSGYTFTRYTMHWVKQRPGQGLEWIEYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 205 - protein, 243 aa, synthetic construct
DIKLQQSGAELARPGASVKMSCKTSGYTFTRYTMHWVKQRPGQGLEWIGEINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 206 - protein, 243 aa, synthetic construct
DIKLQQSGAELARPGASVKMSCKTSGYTFTRYTMHWVKQRPGQGLEWIGYIEPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 207 - protein, 243 aa, synthetic construct
DIKLQQSGAELARPGASVKMSCKTSGYTFTRYTMHWVKQRPGQGLEWIGYINESRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 208 - protein, 243 aa, synthetic construct
DIKLQQSGAELARPGASVKMSCKTSGYTFTRYTMHWVKQRPGQGLEWIGYINPDRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 209 - protein, 243 aa, synthetic construct
DIKLQQSGAELARPGASVKMSCKTSGYTFTRYTMHWVKQRPGQGLEWIGYINPSEGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 210 - protein, 243 aa, synthetic construct
DIKLQQSGAELARPGASVKMSCKTSGYTFTRYTMHWVKQRPGQGLEWIGYINPSRGDTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 211 - protein, 243 aa, synthetic construct
DIKLQQSGAELARPGASVKMSCKTSGYTFTRYTMHWVKQRPGQGLEWIGYINPSRGYTDYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 212 - protein, 243 aa, synthetic construct
DIKLQQSGAELARPGASVKMSCKTSGYTFTRYTMHWVKQRPGQGLEWIGYINPSRGYTNYNQKEKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 213 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVEYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 214 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYDCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 215 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYEARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 216 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCERYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 217 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCAEYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 218 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCAREYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 219 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYEDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 220 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHECLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 221 - protein, 243 aa, synthetic construct
DIKLQQSGAELARPGASVKMSCKTSGYTFTRYTMRWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 222 - protein, 243 aa, synthetic construct
DIKLQQSGAELARPGASVKMSCKTSGYTFTRYTMHWVKQRPGQGLEWRGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 223 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYTDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 224 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARNYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 225 - protein, 243 aa, synthetic construct
DIKLQQSGAELARPGASVKMSCKTSGYTFTRDTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 226 - protein, 243 aa, synthetic construct
DIKLQQSGAELARPGASVKMSCKTSGYTHTRDTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 227 - protein, 243 aa, synthetic construct
DIKLQQSGAELARPGASVKMSCKTSGHTFTRDTMHWVKQRPGQGLEWIGYINPSREYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 228 - protein, 243 aa, synthetic construct
DIKLQQSGAELARPGASVKMSCKTSGYTFTRDTMHWVKQRPGQGLEWIGYHNPSRGYTNYNQKFKDKATLTTDKSSSTAYMQLSSLTSEDSAVYHCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 229 - protein, 243 aa, synthetic construct
DIKLQQSGAELARPGASVKMSCKTSGYTFTRYTMHWVKQRPGQGLEWIGYENPSRGYTNENQKFKDKATLTTDKSSSTAYMQLSSLTSEDSAVYYCARYYDDEYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 230 - protein, 243 aa, synthetic construct
DIKLQQSGAELARPGASVKMSCKTSGYTFTRYTMHWVKQRPGQGLEWIGYINPHREYTNYNQKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 231 - protein, 243 aa, synthetic construct
DIKLQQSGAELARPGASVKMSCKTSGHTFTRYTMHWVKQRPGQGLEWIGYINPSRGYENENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 232 - protein, 243 aa, synthetic construct
DIKLQQSGAELARPGASVKMSCKTSGYEHTRYTMHWVKQRPGQGLEWIGYINPSRGYTNHNQKFKVKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 233 - protein, 243 aa, synthetic construct
DIKLQQSGAELARPGASVKMSCKTSGYTFHRYTMHWVKQRPGQGLEWIGYINPSRGYTNENEKHKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 234 - protein, 243 aa, synthetic construct
DIKLQQSGAELARPGASVKMSCKTSGYTHWRYTMHWVKQRPGQGLEWIGYINPSRGYTNYNQKVEDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 235 - protein, 243 aa, synthetic construct
DIKLQQSGAELARPGASVKMSCKTSGHEFTRYTMHWVKQRPGQGLEWIGYINPSRGYTNYEQKFKDKATLTTDKSSSTAYMQLSSLTSEDSAVYYCARYHDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 236 - protein, 243 aa, synthetic construct
DIKLQQSGAELARPGASVKMSCKTSGYTFTHYTMHWVKQRPGQGLEWIGYINPSRGYENYNQEFHDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 237 - protein, 243 aa, synthetic construct
DIKLQQSGAELARPGASVKMSCKTSGYEFTRYTMHWVKQRPGQGLEWIGYINPSRGYTNYNQKHKHKATLTTDKSSSTAYMQLSSLTSEDSAVYHCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 238 - protein, 243 aa, synthetic construct
DIKLQQSGAELARPGASVKMSCKTSGYTFTRYTMHWVKQRPGQGLEWIGYHNPSRGYTNYNQKVKDKATLTTDKSSSTAYMQLSSLTSEDSAVYYCARYYDHEYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 239 - protein, 243 aa, synthetic construct
DIKLQQSGAELARPGASVKMSCKTSGYTFTHYTMHWVKQRPGQGLEWIGYHNPSRGYTNYEQKFEDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 240 - protein, 243 aa, synthetic construct
DIKLQQSGAELARPGASVKMSCKTSGHTFTRYTMHWVKQRPGQGLEWIGYINPSRGYTNHNQKFKEKATLTTDKSSSTAYMQLSSLTSEDSAVYYCARYEDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 241 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDHSKVASGVPYRFSGSGSGTSYSLTISSMEAEDAATYYCQQWSSNPLTFGAGTKLELK

SEQ ID NO: 242 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSEMNWYQQKSGTSPKRWIYDTSKVASGVPYRFSGSGSGTSYSLTISSMEAEDAATYYCQQWSSNPLTFGAGTKLELK

SEQ ID NO: 243 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWESNPLTFGAGTKLELK

SEQ ID NO: 244 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNHLTFGAGTKLELK

SEQ ID NO: 245 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPETFGAGTKLELK

SEQ ID NO: 246 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHECLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 247 - protein, 243 aa, synthetic construct
DIKLQQSGAELARPGASVKMSCKTSGYTFTRYHMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 248 - protein, 243 aa, synthetic construct
DIKLQQSGAELARPGASVKMSCKTSGYTETRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 249 - protein, 243 aa, synthetic construct
DIKLQQSGAELARPGASVKMSCKTSGYTFTRYTMHWVKQRPGQGLEWIGYINPSRGYTEYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 250 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYEDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 251 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDESKVASGVPYRFSGSGSGTSYSLTISSMEAEDAATYYCQQWSSNPLTFGAGTKLELK

SEQ ID NO: 252 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHECLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 253 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSEMNWYQQKSGTSPKRWIYDTSKVASGVPYRFSGSGSGTSYSLTISSMEAEDAATYYCQQWSSNPLTFGAGTKLELK

SEQ ID NO: 254 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDHSKVASGVPYRFSGSGSGTSYSLTISSMEAEDAATYYCQQWSSNPLTFGAGTKLELK

SEQ ID NO: 255 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYENWYQQKSGTSPKRWIYDTSKVASGVPYRFSGSGSGTSYSLTISSMEAEDAATYYCQQWSSNPLTFGAGTKLELK

SEQ ID NO: 256 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHHCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 257 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCAREYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 258 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARETDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 259 - protein, 243 aa, synthetic construct
DIKLQQSGAELARPGASVKMSCKTSGYTFTRYEMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 260 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSEMNWYQQKSGTSPKRWIYDTSKVASGVPYRFSGSGSGTSYSLTISSMEAEDAATYYCQQWSSNPLTFGAGTKLELK

SEQ ID NO: 261 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDHSKVASGVPYRFSGSGSGTSYSLTISSMEAEDAATYYCQQWSSNPLTFGAGTKLELK

SEQ ID NO: 262 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARHTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 263 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCEQWSSNPLTFGAGTKLELK

SEQ ID NO: 264 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSEPLTFGAGTKLELK

SEQ ID NO: 265 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSHMNWYQQKSGTSPKRWIYDTSKVASGVPYRFSGSGSGTSYSLTISSMEAEDAATYYCQQWSSNPLTFGAGTKLELK

SEQ ID NO: 266 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVEYMNWYQQKSGTSPKRWIYDTSKVASGVPYRFSGSGSGTSYSLTISSMEAEDAATYYCQQWSSNPLTFGAGTKLELK

SEQ ID NO: 267 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQESSNPLTFGAGTKLELK

SEQ ID NO: 268 - protein, 243 aa, synthetic construct
DIKLQQSGAELARPGASVKMSCKTSGYTFTRYEMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 269 - protein, 243 aa, synthetic construct
DIKLQQSGAELARPGASVKMSCKTSGYTETRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 270 - protein, 243 aa, synthetic construct
DIKLQQSGAELARPGASVKMSCKTSGYTFTRYTMHWVKQRPGQGLEWIGYINPSRGEHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 271 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYHNWYQQKSGTSPKRWIYDTSKVASGVPYRFSGSGSGTSYSLTISSMEAEDAATYYCQQWSSNPLTFGAGTKLELK

SEQ ID NO: 272 - protein, 243 aa, synthetic construct
DIKLQQSGAELARPGASVKMSCKTSGYTFTRYTMHWVKQRPGQGLEWIGYINPSRGETNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 273 - protein, 243 aa, synthetic construct
DIKLQQSGAELARPGASVKMSCKTSGYTFTHYEMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 274 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIEDTSKVASGVPYRFSGSGSGTSYSLTISSMEAEDAATYYCQQWSSNPLTFGAGTKLELK

SEQ ID NO: 275 - protein, 243 aa, synthetic construct
DIKLQQSGAELARPGASVKMSCKTSGYTFTHYTMHWVKQRPGQGLEWIGYINPSRGYTEYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 276 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARHYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 277 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQHSSNPLTFGAGTKLELK

SEQ ID NO: 278 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSHMNWYQQKSGTSPKRWIYDTSKVASGVPYRFSGSGSGTSYSLTISSMEAEDAATYYCQQWSSNPLTFGAGTKLELK

SEQ ID NO: 279 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNHLTFGAGTKLELK

SEQ ID NO: 280 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPHTFGAGTKLELK

SEQ ID NO: 281 - protein, 243 aa, synthetic construct
DIKLQQSGAELARPGASVKMSCKTSGYTFTRYEMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 282 - protein, 243 aa, synthetic construct
DIKLQQSGAELARPGASVKMSCKTSGYTFTRYTMHWVKQRPGQGLEWIGYINPSRGHHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 283 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCHQWSSNPLTFGAGTKLELK

SEQ ID NO: 284 - protein, 243 aa, synthetic construct
DIKLQQSGAELARPGASVKMSCKTSGYTFTRETMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 285 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCAREYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 286 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSHMNWYQQKSGTSPKRWIYDTSKVASGVPYRFSGSGSGTSYSLTISSMEAEDAATYYCQQWSSNPLTFGAGTKLELK

SEQ ID NO: 287 - protein, 243 aa, synthetic construct
DIKLQQSGAELARPGASVKMSCKTSGYTFERYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 288 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHECLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 289 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDDHYCLDYWGQGTTLTVSSVEGGSGGSGGSGGSGGVDDIQLTQSPAIMSASPGEKVTMTCRASSSVSYMNWYQQKSGTSPKRWIYHTSKVASGVPYRFSGSGSGTSYSLTISSMEAEDAATYYCQQWSSNPLTFGAGTKLELK

SEQ ID NO: 290 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNELTFGAGTKLELK

SEQ ID NO: 291 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYETSKVASGVPYRFSGSGSGTSYSLTISSMEAEDAATYYCQQWSSNPLTFGAGTKLELK

SEQ ID NO: 292 - protein, 243 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARHYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 293 - protein, 241 aa, synthetic construct
QIQLVQSGAEVAKPGASVKVSCKASGYTFTRYTMHWVRQRPGQGLEWIGYINPSRGYTNYNQKFKDRATLITDKSTSTAYMELSSLTSEDTAVYYCARYYDDHYCLDYWGQGTIVIVSSVEGGSGGSGGSGGSVDDIQLTQSPSSLSASVGDRVTITCRASSSVSYMNWYQQKSGTAPKRWIYDTSKVASGVPYRFSGSGSGTSYTLTISSLQPEDAATYYCQQWSSNPLTFGGGTKVEIK

SEQ ID NO: 294 - protein, 241 aa, synthetic construct
QIQLVQSGAEVAKPGASVKVSCKASGYTFTRYTMHWVRQRPGQGLEWIGYINPSRGYTNYNQKFKDRATLITDKSTSTAYMELSSLTSEDTAVYYCARYYDDHYCLDYWGQGTIVIVSSVEGGSGGSGGSGGSVDDIQLTQPPSSLSASVGDRVTITCRASSSVHYMNWYQQKSGTAPKRWIYDTSKVASGVPYRFSGSGSGTSYTLTISSLQPEDAATYYCQQWSSNPLTFGGGTKVEIK

SEQ ID NO: 295 - protein, 241 aa, synthetic construct
QIQLVQSGAEVAKPGASVKVSCKASGYTFTRYTMHWVRQRPGQGLEWIGYINPSHGYTNYNQKFKDRATLITDKSTSTAYMELSSLTSEDTAVYYCARYYDDHYCLDYWGQGTIVIVSSVEGGSGGSGGSGGSVDDIQLTQSPSSLSASVGDRVTITCRASSSVSYMNWYQQKSGTAPKRWIYDTSKVASGVPYRFSGSGSGTSYTLTISSLQPEDAATYYCQQWSSNPLTFGGGTKVEIK

SEQ ID NO: 296 - protein, 241 aa, synthetic construct
QIQLVQSGAEVAKPGASVKVSCKASGYTFTRYTMHWVRQRPGQGLEWIGYINPSRGYTNYNQKFKDRATLITDKSTSTAYMELSSLTSEDTAVYYCARYYDDHYCLDYWGQGTIVIVSSVEGGSGGSGGSGGSVDDIQLTQSPSSLSASVGDRVTITCRASSSVHYMNWYQQKSGTAPKRWIYDTSKVASGVPYRFSGSGSGTSYTLTISSLQPEDAATYYCQQWSSNPLTFGGGTKVEIK

SEQ ID NO: 297 - protein, 119 aa, synthetic construct
DIKLQQSGAELARPGASVKMSCKTSGYTFTRYTMHWVKQRPGQGLEWIGYINPSRGYHNHNQKFKDKATLTTDKSSSTAYMQLSSLTSEDSAVYYCARYYDDHYCLDYWGQGTTLTVSS

SEQ ID NO: 298 - protein, 119 aa, synthetic construct
DIKLQQSGAELARPGASVKMSCKTSGYTFTRYTMHWVKQRPGQGLEWIGYINPSRGYHHYNQHFKDKATLTTDKSSSTAYMQLSSLTSEDSAVYYCARYYDDHYCLDYWGQGTTLTVSS

SEQ ID NO: 299 - protein, 119 aa, synthetic construct
DIKLQQSGAELARPGASVKMSCKTSGYTFTRHTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 300 - protein, 119 aa, synthetic construct
DIKLQQSGAELARPGASVKMSCKTSGYEFTRYTMHWVKQRPGQGLEWIGYINPSRGYTHYNQKFKDKATLTTDKSSSTAYMQLSSLTSEDSAVYYCARYYDDHYCLDYWGQGTTLTVSS

SEQ ID NO: 301 - protein, 119 aa, synthetic construct
DIKLQQSGAELARPGASVKMSCKTSGYTFTRYTMHWVKQRPGQGLEWIGYINPHREYTNYNQKFKDKATLTTDKSSSTAYMQLSSLTSEDSAVYYCARYYDDHYCLDYWGQGTTLTVSS

SEQ ID NO: 302 - protein, 119 aa, synthetic construct
DIKLQQSGAELARPGASVKMSCKTSGYTFTRYTMHWVKQRPGQGLEWIGYINPHRGYEHYNQKFKEKATLTTDKSSSTAYMQLSSLTSEDSAVYYCARYYDDHYCLDYWGQGTTLTVSS

SEQ ID NO: 303 - protein, 119 aa, synthetic construct
DIKLQQSGAELARPGASVKMSCKTSGYGFTHYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 304 - protein, 119 aa, synthetic construct
DIKLQQSGAELARPGASVKMSCKTSGYGFTRYTMHWVKQRPGQGLEWIGYINPHRGYTNYNQKFKDKATLTTDKSSSTAYMQLSSLTSEDSAVYYCARYYDDHYCLDYWGQGTTLTVSS

SEQ ID NO: 305 - protein, 119 aa, synthetic construct
DIKLQQSGAELARPGASVKMSCKTSGYTFTRYTMHWVKQRPGQGLEWIGYINPSRGYTNYNQHFHDKATLTTDKSSSTAYMQLSSLTSEDSAVYYCARYYDDHYCLDYWGQGTTLTVSS

SEQ ID NO: 306 - protein, 119 aa, synthetic construct
DIKLQQSGAELARPGASVKMSCKTSGYTFTRYTMHWVKQRPGQGLEWIGYINPSRGYENYHQKFKHKATLTTDKSSSTAYMQLSSLTSEDSAVYYCARYYDDHYCLDYWGQGTTLTVSS

SEQ ID NO: 307 - protein, 119 aa, synthetic construct
DIKLQQSGAELARPGASVKMSCKTSGYTFTRYTMHWVKQRPGQGLEWIGYINPSRGYTNYNQKFHDKATLTTDKSSSTAYMQLSSLTSEDSAVYYCARYYDDHYCLDYWGQGTTLTVSS

SEQ ID NO: 308 - protein, 119 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDEYCLDYWGQGTTLTVSS

SEQ ID NO: 309 - protein, 119 aa, synthetic construct
DIKLQQSGAELARPGASVKMSCKTSGYHFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 310 - protein, 119 aa, synthetic construct
DIKLQQSGAELARPGASVKMSCKTSGYTFTRYTMHWVKQRPGQGLEWIGYINPSRGYTHENQHFKEKATLTTDKSSSTAYMQLSSLTSEDSAVYYCARYYDDHYCLDYWGQGTTLTVSS

SEQ ID NO: 311 - protein, 119 aa, synthetic construct
DIKLQQSGAELARPGASVKMSCKTSGYGFTRYTMHWVKQRPGQGLEWIGYINPSRGYTNYNQKFKVKATLTTDKSSSTAYMQLSSLTSEDSAVYYCARYTDDHYCLDYWGQGTTLTVSS

SEQ ID NO: 312 - protein, 119 aa, synthetic construct
DIKLQQSGAELARPGASVKMSCKTSGYTFWRYTMHWVKQRPGQGLEWIGYINPSRHYTNYNQKFKDKATLTTDKSSSTAYMQLSSLTSEDSAVYYCARYYDDHYCLDYWGQGTTLTVSS

SEQ ID NO: 313 - protein, 119 aa, synthetic construct
DIKLQQSGAELARPGASVKMSCKTSGHTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 314 - protein, 119 aa, synthetic construct
DIKLQQSGAELARPGASVKMSCKTSGYTFTRYTQHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 315 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYEDDHYCLDYWGQGTTLTVSS

SEQ ID NO: 316 - protein, 119 aa, synthetic construct
DIKLQQSGAELARPGASVKMSCKTSGYTFTRYTMHWVKQRPGQGLEWIGYINPSRGYTNYHQKFKDKATLTTDKSSSTAYMQLSSLTSEDSAVYYCARYYDDHYCLDYWGQGTTLTVSS

SEQ ID NO: 317 - protein, 119 aa, synthetic construct
DIKLQQSGAELARPGASVKMSCKTSGYTFTRYTMHWVKQRPGQGLEWIGYINPSRNYTNYNQKFKDKATLTTDKSSSTAYMQLSSLTSEDSAVYYCARYYDDHYCLDYWGQGTTLTVSS

SEQ ID NO: 318 - protein, 119 aa, synthetic construct
DIKLQQSGAELARPGASVKMSCKTSGYTHTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 319 - protein, 119 aa, synthetic construct
DIKLQQSGAELARPGASVKMSCKTSGYTFTRYTMHWVKQRPGQGLEWIGYINPSRGYTNYNQHFKDKATLTTDKSSSTAYMQLSSLTSEDSAVYYCARYYDDHYCLDYWGQGTTLTVSS

SEQ ID NO: 320 - protein, 119 aa, synthetic construct
DIKLQQSGAELARPGASVKMSCKTSGYTFTRYTMHWVKQRPGQGLEWIGYINPSRGYTNYNEKFKDKATLTTDKSSSTAYMQLSSLTSEDSAVYYCARYYDDHYCLDYWGQGTTLTVSS

SEQ ID NO: 321 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDHHYCLDYWGQGTTLTVSS

SEQ ID NO: 322 - protein, 119 aa, synthetic construct
DIKLQQSGAELARPGASVKMSCKTSGYTFTRYTMHWVKQRPGQGLEWIGYINPSRGYTNHNQKFKDKATLTTDKSSSTAYMQLSSLTSEDSAVYYCARYYDDHYCLDYWGQGTTLTVSS

SEQ ID NO: 323 - protein, 119 aa, synthetic construct
DIKLQQSGAELARPGASVKMSCKTSGYGFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 324 - protein, 119 aa, synthetic construct
DIKLQQSGAELARPGASVKMSCKTSGYTFWRYTMHWVKQRPGQGLEWIGYINPSRGYTNYNQKFEDKATLTTDKSSSTAYMQLSSLTSEDSAVYYCARYYDDHYCLDYWGQGTTLTVSS

SEQ ID NO: 325 - protein, 119 aa, synthetic construct
DIKLQQSGAELARPGASVKMSCKTSGYTFTRYTMHWVKQRPGQGLEWIGYINPSRGYTNYNQKFEDKATLTTDKSSSTAYMQLSSLTSEDSAVYYCARYYDDHYCLDYWGQGTTLTVSS

SEQ ID NO: 326 - protein, 119 aa, synthetic construct
DIKLQQSGAELARPGASVKMSCKTSGYTHTRYTMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 327 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYHCARYYDDHYCLDYWGQGTTLTVSS

SEQ ID NO: 328 - protein, 119 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDHHYCLDYWGQGTTLTVSS

SEQ ID NO: 329 - protein, 119 aa, synthetic construct
DIKLQQSGAELARPGASVKMSCKTSGYTFTRYTMHWVKQRPGQGLEWIGYINPSRGYHNYNQKFKVKATLTTDKSSSTAYMQLSSLTSEDSAVYYCARYYDDHYCLDYWGQGTTLTVSS

SEQ ID NO: 330 - protein, 119 aa, synthetic construct
DIKLQQSGAELARPGASVKMSCKTSGYTFTRYTMHWVKQRPGQGLEWIGYINPSRGYTNYNQKHKDKATLTTDKSSSTAYMQLSSLTSEDSAVYYCARYYDDHYCLDYWGQGTTLTVSS

SEQ ID NO: 331 - protein, 119 aa, synthetic construct
DIKLQQSGAELARPGASVKMSCKTSGYTFTRYTHHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 332 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDEHYCLDYWGQGTTLTVSS

SEQ ID NO: 333 - protein, 119 aa, synthetic construct
DIKLQQSGAELARPGASVKMSCKTSGYTFTRYTMHWVKQRPGQGLEWIGYINPSRGYTNYNQEFKDKATLTTDKSSSTAYMQLSSLTSEDSAVYYCARYYDDHYCLDYWGQGTTLTVSS

SEQ ID NO: 334 - protein, 119 aa, synthetic construct
DIKLQQSGAELARPGASVKMSCKTSGYTFTRYTMHWVKQRPGQGLEWIGYINPSRHYTNYNQKFKDKATLTTDKSSSTAYMQLSSLTSEDSAVYYCARYYDDHYCLDYWGQGTTLTVSS

SEQ ID NO: 335 - protein, 119 aa, synthetic construct
DIKLQQSGAELARPGASVKMSCKTSGYTFWRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 336 - protein, 119 aa, synthetic construct
DIKLQQSGAELARPGASVKMSCKTSGYTFTRYTMHWVKQRPGQGLEWIGYINPSRGYHNENQKFKDKATLTTDKSSSTAYMQLSSLTSEDSAVYYCARYYDDHYCLDYWGQGTTLTVSS

SEQ ID NO: 337 - protein, 119 aa, synthetic construct
DIKLQQSGAELARPGASVKMSCKTSGYGFTRYTMHWVKQRPGQGLEWIGYINPSRNYTNYNQEFKHKATLTTDKSSSTAYMQLSSLTSEDSAVYYCARYYDDHYCLDYWGQGTTLTVSS

SEQ ID NO: 338 - protein, 119 aa, synthetic construct
DIKLQQSGAELARPGASVKMSCKTSGYTFTRYTMHWVKQRPGQGLEWIGYINPSRHYTNYNQKHKDKATLTTDKSSSTAYMQLSSLTSEDSAVYYCARYYDDHYCLDYWGQGTTLTVSS

SEQ ID NO: 339 - protein, 119 aa, synthetic construct
DIKLQQSGAELARPGASVKMSCKTSGYTHTRYTMHWVKQRPGQGLEWIGYINPSRGYTNYNEKVKDKATLTTDKSSSTAYMQLSSLTSEDSAVYYCARYYDDHYCLDYWGQGTTLTVSS

SEQ ID NO: 340 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYSARYYDDHYCLDYWGQGTTLTVSS

SEQ ID NO: 341 - protein, 119 aa, synthetic construct
DIKLQQSGAELARPGASVKMSCKTSGYTFTRYTMHWVKQRPGQGLEWIGYINPHRGYTNYHQKHKDKATLTTDKSSSTAYMQLSSLTSEDSAVYYCARYYDDEYCLDYWGQGTTLTVSS

SEQ ID NO: 342 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDEYCLDYWGQGTTLTVSS

SEQ ID NO: 343 - protein, 119 aa, synthetic construct
DIKLQQSGAELARPGASVKMSCKTSGYTFTRYTMHWVKQRPGQGLEWIGYINPSRGYTNYNHKFKDKATLTTDKSSSTAYMQLSSLTSEDSAVYYCARYYDDHYCLDYWGQGTTLTVSS

SEQ ID NO: 344 - protein, 119 aa, synthetic construct
DIKLQQSGAELARPGASVKMSCKTSGYTFTRYTMHWVKQRPGQGLEWIGYENPSRGYTNYNQKFKDKATLTTDKSSSTAYMQLSSLTSEDSAVYYCARYYDDHYCLDYWGQGTTLTVSS

SEQ ID NO: 345 - protein, 119 aa, synthetic construct
DIKLQQSGAELARPGASVKMSCKTSGYTFTRYTMHWVKQRPGQGLEWIGYINPSRGYTNYNHKVKDKATLTTDKSSSTAYMQLSSLTSEDSAVYYCARYYDDHYCLDYWGQGTTLTVSS

SEQ ID NO: 346 - protein, 119 aa, synthetic construct
DIKLQQSGAELARPGASVKMSCKTSGYTFTRYTMHWVKQRPGQGLEWIGYINPSRGYTHYNQKFKDKATLTTDKSSSTAYMQLSSLTSEDSAVYYCARYYDDHYCLDYWGQGTTLTVSS

SEQ ID NO: 347 - protein, 119 aa, synthetic construct
DIKLQQSGAELARPGASVKMSCKTSGYTFTRYTMHWVKQRPGQGLEWIGYINPSRGYTNYEQKFKDKATLTTDKSSSTAYMQLSSLTSEDSAVYYCARYYDDHYCLDYWGQGTTLTVSS

SEQ ID NO: 348 - protein, 119 aa, synthetic construct
DIKLQQSGAELARPGASVKMSCKTSGYTFTRYTMHWVKQRPGQGLEWIGYINPSRGYTNYNQKFKEKATLTTDKSSSTAYMQLSSLTSEDSAVYYCARYYDDHYCLDYWGQGTTLTVSS

SEQ ID NO: 349 - protein, 119 aa, synthetic construct
DIKLQQSGAELARPGASVKMSCKTSGYGFTRYTMHWVKQRPGQGLEWIGYINPSRGYTNYNQKFKDKATLTTDKSSSTAYMQLSSLTSEDSAVYYCARYHDDHYCLDYWGQGTTLTVSS

SEQ ID NO: 350 - protein, 119 aa, synthetic construct
DIKLQQSGAELARPGASVKMSCKTSGYTFTRYTMHWVKQRPGQGLEWIGYINPSRGYTNENQEFKDKATLTTDKSSSTAYMQLSSLTSEDSAVYYCARYYDEHYCLDYWGQGTTLTVSS

SEQ ID NO: 351 - protein, 119 aa, synthetic construct
DIKLQQSGAELARPGASVKMSCKTSGYTFTRYTMHWVKQRPGQGLEWIGYINPSRGYTNYNQKFKHKATLTTDKSSSTAYMQLSSLTSEDSAVYYCARYYDDHYCLDYWGQGTTLTVSS

SEQ ID NO: 352 - protein, 119 aa, synthetic construct
DIKLQQSGAELARPGASVKMSCKTSGYEFTRHTMHWVKQRPGQGLEWIGYINPSRGYTNYNHKFKDKATLTTDKSSSTAYMQLSSLTSEDSAVYYCARYYDDHYCLDYWGQGTTLTVSS

SEQ ID NO: 353 - protein, 119 aa, synthetic construct
DIKLQQSGAELARPGASVKMSCKTSGYTFTRYTMHWVKQRPGQGLEWIGYINPSRGYTNYNQKFKVKATLTTDKSSSTAYMQLSSLTSEDSAVYYCARYYDDHYCLDYWGQGTTLTVSS

SEQ ID NO: 354 - protein, 119 aa, synthetic construct
DIKLQQSGAELARPGASVKMSCKTSGYTFTRYTMHWVKQRPGQGLEWIGYINPHRGYTNYNQKFKDKATLTTDKSSSTAYMQLSSLTSEDSAVYYCARYYDDHYCLDYWGQGTTLTVSS

SEQ ID NO: 355 - protein, 119 aa, synthetic construct
DIKLQQSGAELARPGASVKMSCKTSGYTFTRDTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 356 - protein, 119 aa, synthetic construct
DIKLQQSGAELARPGASVKMSCKTSGYEFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 357 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYTDHHYCLDYWGQGTTLTVSS

SEQ ID NO: 358 - protein, 119 aa, synthetic construct
DIKLQQSGAELARPGASVKMSCKTSGYTFTRYTMHWVKQRPGQGLEWIGYINPHRGYTNYNQKFKHKATLTTDKSSSTAYMQLSSLTSEDSAVYHCARYYDDHYCLDYWGQGTTLTVSS

SEQ ID NO: 359 - protein, 119 aa, synthetic construct
DIKLQQSGAELARPGASVKMSCKTSGYTFTRYTMHWVKQRPGQGLEWIGYINPSRGYTNYNQKVKDKATLTTDKSSSTAYMQLSSLTSEDSAVYYCARYYDDHYCLDYWGQGTTLTVSS

SEQ ID NO: 360 - protein, 119 aa, synthetic construct
DIKLQQSGAELARPGASVKMSCKTSGYGFTRYTMHWVKQRPGQGLEWIGYINPSRGYTNYEHKVKDKATLTTDKSSSTAYMQLSSLTSEDSAVYYCARYYDDHYCLDYWGQGTTLTVSS

SEQ ID NO: 361 - protein, 119 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDHHYCLDYWGQGTTLTVSS

SEQ ID NO: 362 - protein, 119 aa, synthetic construct
DIKLQQSGAELARPGASVKMSCKTSGYTFTRYHMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 363 - protein, 119 aa, synthetic construct
DIKLQQSGAELARPGASVKMSCKTSGYTFTRYTMHHVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 364 - protein, 119 aa, synthetic construct
DIKLQQSGAELARPGASVKMSCKTSGYTFTRYTMHWVKQRPGQGLEWHGYINPSRGYTNYNQKFKDKATLTTDKSSSTAYMQLSSLTSEDSAVYYCARYYDDHYCLDYWGQGTTLTVSS

SEQ ID NO: 365 - protein, 119 aa, synthetic construct
DIKLQQSGAELARPGASVKMSCKTSGYTFTRYTMHWVKQRPGQGLEWIHYINPSRGYTNYNQKFKDKATLTTDKSSSTAYMQLSSLTSEDSAVYYCARYYDDHYCLDYWGQGTTLTVSS

SEQ ID NO: 366 - protein, 119 aa, synthetic construct
DIKLQQSGAELARPGASVKMSCKTSGYTFTRYTMHWVKQRPGQGLEWIGHINPSRGYTNYNQKFKDKATLTTDKSSSTAYMQLSSLTSEDSAVYYCARYYDDHYCLDYWGQGTTLTVSS

SEQ ID NO: 367 - protein, 119 aa, synthetic construct
DIKLQQSGAELARPGASVKMSCKTSGYTFTRYTMHWVKQRPGQGLEWIGYINHSRGYTNYNQKFKDKATLTTDKSSSTAYMQLSSLTSEDSAVYYCARYYDDHYCLDYWGQGTTLTVSS

SEQ ID NO: 368 - protein, 119 aa, synthetic construct
DIKLQQSGAELARPGASVKMSCKTSGYTFTRYTMHWVKQRPGQGLEWIGYINPSRGHTNYNQKFKDKATLTTDKSSSTAYMQLSSLTSEDSAVYYCARYYDDHYCLDYWGQGTTLTVSS

SEQ ID NO: 369 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVHYCARYYDDHYCLDYWGQGTTLTVSS

SEQ ID NO: 370 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYHARYYDDHYCLDYWGQGTTLTVSS

SEQ ID NO: 371 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCHRYYDDHYCLDYWGQGTTLTVSS

SEQ ID NO: 372 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCAHYYDDHYCLDYWGQGTTLTVSS

SEQ ID NO: 373 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARHYDDHYCLDYWGQGTTLTVSS

SEQ ID NO: 374 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYHDHYCLDYWGQGTTLTVSS

SEQ ID NO: 375 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHHCLDYWGQGTTLTVSS

SEQ ID NO: 376 - protein, 119 aa, synthetic construct
DIKLQQSGAELARPGASVKMSCKTSGDTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 377 - protein, 119 aa, synthetic construct
DIKLQQSGAELARPGASVKMSCKTSGYTDTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 378 - protein, 119 aa, synthetic construct
DIKLQQSGAELARPGASVKMSCKTSGYTFERYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 379 - protein, 119 aa, synthetic construct
DIKLQQSGAELARPGASVKMSCKTSGYTFTDYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 380 - protein, 119 aa, synthetic construct
DIKLQQSGAELARPGASVKMSCKTSGYTFTRYEMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 381 - protein, 119 aa, synthetic construct
DIKLQQSGAELARPGASVKMSCKTSGYTFTRYTEHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 382 - protein, 119 aa, synthetic construct
DIKLQQSGAELARPGASVKMSCKTSGYTFTRYTMDWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 383 - protein, 119 aa, synthetic construct
DIKLQQSGAELARPGASVKMSCKTSGYTFTRYTMHEVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 384 - protein, 119 aa, synthetic construct
DIKLQQSGAELARPGASVKMSCKTSGYTFTRYTMHWVKQRPGQGLEWDGYINPSRGYTNYNQKFKDKATLTTDKSSSTAYMQLSSLTSEDSAVYYCARYYDDHYCLDYWGQGTTLTVSS

SEQ ID NO: 385 - protein, 119 aa, synthetic construct
DIKLQQSGAELARPGASVKMSCKTSGYTFTRYTMHWVKQRPGQGLEWIEYINPSRGYTNYNQKFKDKATLTTDKSSSTAYMQLSSLTSEDSAVYYCARYYDDHYCLDYWGQGTTLTVSS

SEQ ID NO: 386 - protein, 119 aa, synthetic construct
DIKLQQSGAELARPGASVKMSCKTSGYTFTRYTMHWVKQRPGQGLEWIGEINPSRGYTNYNQKFKDKATLTTDKSSSTAYMQLSSLTSEDSAVYYCARYYDDHYCLDYWGQGTTLTVSS

SEQ ID NO: 387 - protein, 119 aa, synthetic construct
DIKLQQSGAELARPGASVKMSCKTSGYTFTRYTMHWVKQRPGQGLEWIGYIEPSRGYTNYNQKFKDKATLTTDKSSSTAYMQLSSLTSEDSAVYYCARYYDDHYCLDYWGQGTTLTVSS

SEQ ID NO: 388 - protein, 119 aa, synthetic construct
DIKLQQSGAELARPGASVKMSCKTSGYTFTRYTMHWVKQRPGQGLEWIGYINESRGYTNYNQKFKDKATLTTDKSSSTAYMQLSSLTSEDSAVYYCARYYDDHYCLDYWGQGTTLTVSS

SEQ ID NO: 389 - protein, 119 aa, synthetic construct
DIKLQQSGAELARPGASVKMSCKTSGYTFTRYTMHWVKQRPGQGLEWIGYINPDRGYTNYNQKFKDKATLTTDKSSSTAYMQLSSLTSEDSAVYYCARYYDDHYCLDYWGQGTTLTVSS

SEQ ID NO: 390 - protein, 119 aa, synthetic construct
DIKLQQSGAELARPGASVKMSCKTSGYTFTRYTMHWVKQRPGQGLEWIGYINPSEGYTNYNQKFKDKATLTTDKSSSTAYMQLSSLTSEDSAVYYCARYYDDHYCLDYWGQGTTLTVSS

SEQ ID NO: 391 - protein, 119 aa, synthetic construct
DIKLQQSGAELARPGASVKMSCKTSGYTFTRYTMHWVKQRPGQGLEWIGYINPSRGDTNYNQKFKDKATLTTDKSSSTAYMQLSSLTSEDSAVYYCARYYDDHYCLDYWGQGTTLTVSS

SEQ ID NO: 392 - protein, 119 aa, synthetic construct
DIKLQQSGAELARPGASVKMSCKTSGYTFTRYTMHWVKQRPGQGLEWIGYINPSRGYTDYNQKFKDKATLTTDKSSSTAYMQLSSLTSEDSAVYYCARYYDDHYCLDYWGQGTTLTVSS

SEQ ID NO: 393 - protein, 119 aa, synthetic construct
DIKLQQSGAELARPGASVKMSCKTSGYTFTRYTMHWVKQRPGQGLEWIGYINPSRGYTNYNQKEKDKATLTTDKSSSTAYMQLSSLTSEDSAVYYCARYYDDHYCLDYWGQGTTLTVSS

SEQ ID NO: 394 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVEYCARYYDDHYCLDYWGQGTTLTVSS

SEQ ID NO: 395 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYDCARYYDDHYCLDYWGQGTTLTVSS

SEQ ID NO: 396 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYEARYYDDHYCLDYWGQGTTLTVSS

SEQ ID NO: 397 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCERYYDDHYCLDYWGQGTTLTVSS

SEQ ID NO: 398 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCAEYYDDHYCLDYWGQGTTLTVSS

SEQ ID NO: 399 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCAREYDDHYCLDYWGQGTTLTVSS

SEQ ID NO: 400 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYEDHYCLDYWGQGTTLTVSS

SEQ ID NO: 401 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHECLDYWGQGTTLTVSS

SEQ ID NO: 402 - protein, 119 aa, synthetic construct
DIKLQQSGAELARPGASVKMSCKTSGYTFTRYTMRWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 403 - protein, 119 aa, synthetic construct
DIKLQQSGAELARPGASVKMSCKTSGYTFTRYTMHWVKQRPGQGLEWRGYINPSRGYTNYNQKFKDKATLTTDKSSSTAYMQLSSLTSEDSAVYYCARYYDDHYCLDYWGQGTTLTVSS

SEQ ID NO: 404 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYTDHYCLDYWGQGTTLTVSS

SEQ ID NO: 405 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARNYDDHYCLDYWGQGTTLTVSS

SEQ ID NO: 406 - protein, 119 aa, synthetic construct
DIKLQQSGAELARPGASVKMSCKTSGYTFTRDTMHWVKQRPGQGLEWIGYINPSRGYTNENQKFKDKATLTTDKSSSTAYMQLSSLTSEDSAVYYCARYYDDHYCLDYWGQGTTLTVSS

SEQ ID NO: 407 - protein, 119 aa, synthetic construct
DIKLQQSGAELARPGASVKMSCKTSGYTHTRDTMHWVKQRPGQGLEWIGYINPSRGYTNENQKFKDKATLTTDKSSSTAYMQLSSLTSEDSAVYYCARYYDDHYCLDYWGQGTTLTVSS

SEQ ID NO: 408 - protein, 119 aa, synthetic construct
DIKLQQSGAELARPGASVKMSCKTSGHTFTRDTMHWVKQRPGQGLEWIGYINPSREYTNYNQKFKDKATLTTDKSSSTAYMQLSSLTSEDSAVYYCARYYDDHYCLDYWGQGTTLTVSS

SEQ ID NO: 409 - protein, 119 aa, synthetic construct
DIKLQQSGAELARPGASVKMSCKTSGYTFTRDTMHWVKQRPGQGLEWIGYHNPSRGYTNYNQKFKDKATLTTDKSSSTAYMQLSSLTSEDSAVYHCARYYDDHYCLDYWGQGTTLTVSS

SEQ ID NO: 410 - protein, 119 aa, synthetic construct
DIKLQQSGAELARPGASVKMSCKTSGYTFTRYTMHWVKQRPGQGLEWIGYENPSRGYTNENQKFKDKATLTTDKSSSTAYMQLSSLTSEDSAVYYCARYYDDEYCLDYWGQGTTLTVSS

SEQ ID NO: 411 - protein, 119 aa, synthetic construct
DIKLQQSGAELARPGASVKMSCKTSGYTFTRYTMHWVKQRPGQGLEWIGYINPHREYTNYNQKVKDKATLTTDKSSSTAYMQLSSLTSEDSAVYYCARYYDDHYCLDYWGQGTTLTVSS

SEQ ID NO: 412 - protein, 119 aa, synthetic construct
DIKLQQSGAELARPGASVKMSCKTSGHTFTRYTMHWVKQRPGQGLEWIGYINPSRGYENENQKFKDKATLTTDKSSSTAYMQLSSLTSEDSAVYYCARYYDDHYCLDYWGQGTTLTVSS

SEQ ID NO: 413 - protein, 119 aa, synthetic construct
DIKLQQSGAELARPGASVKMSCKTSGYEHTRYTMHWVKQRPGQGLEWIGYINPSRGYTNHNQKFKVKATLTTDKSSSTAYMQLSSLTSEDSAVYYCARYYDDHYCLDYWGQGTTLTVSS

SEQ ID NO: 414 - protein, 119 aa, synthetic construct
DIKLQQSGAELARPGASVKMSCKTSGYTFHRYTMHWVKQRPGQGLEWIGYINPSRGYTNENEKHKDKATLTTDKSSSTAYMQLSSLTSEDSAVYYCARYYDDHYCLDYWGQGTTLTVSS

SEQ ID NO: 415 - protein, 119 aa, synthetic construct
DIKLQQSGAELARPGASVKMSCKTSGYTHWRYTMHWVKQRPGQGLEWIGYINPSRGYTNYNQKVEDKATLTTDKSSSTAYMQLSSLTSEDSAVYYCARYYDDHYCLDYWGQGTTLTVSS

SEQ ID NO: 416 - protein, 119 aa, synthetic construct
DIKLQQSGAELARPGASVKMSCKTSGHEFTRYTMHWVKQRPGQGLEWIGYINPSRGYTNYEQKFKDKATLTTDKSSSTAYMQLSSLTSEDSAVYYCARYHDDHYCLDYWGQGTTLTVSS

SEQ ID NO: 417 - protein, 119 aa, synthetic construct
DIKLQQSGAELARPGASVKMSCKTSGYTFTHYTMHWVKQRPGQGLEWIGYINPSRGYENYNQEFHDKATLTTDKSSSTAYMQLSSLTSEDSAVYYCARYYDDHYCLDYWGQGTTLTVSS

SEQ ID NO: 418 - protein, 119 aa, synthetic construct
DIKLQQSGAELARPGASVKMSCKTSGYEFTRYTMHWVKQRPGQGLEWIGYINPSRGYTNYNQKHKHKATLTTDKSSSTAYMQLSSLTSEDSAVYHCARYYDDHYCLDYWGQGTTLTVSS

SEQ ID NO: 419 - protein, 119 aa, synthetic construct
DIKLQQSGAELARPGASVKMSCKTSGYTFTRYTMHWVKQRPGQGLEWIGYHNPSRGYTNYNQKVKDKATLTTDKSSSTAYMQLSSLTSEDSAVYYCARYYDHEYCLDYWGQGTTLTVSS

SEQ ID NO: 420 - protein, 119 aa, synthetic construct
DIKLQQSGAELARPGASVKMSCKTSGYTFTHYTMHWVKQRPGQGLEWIGYHNPSRGYTNYEQKFEDKATLTTDKSSSTAYMQLSSLTSEDSAVYYCARYYDDHYCLDYWGQGTTLTVSS

SEQ ID NO: 421 - protein, 119 aa, synthetic construct
DIKLQQSGAELARPGASVKMSCKTSGHTFTRYTMHWVKQRPGQGLEWIGYINPSRGYTNHNQKFKEKATLTTDKSSSTAYMQLSSLTSEDSAVYYCARYEDDHYCLDYWGQGTTLTVSS

SEQ ID NO: 422 - protein, 119 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHECLDYWGQGTTLTVSS

SEQ ID NO: 423 - protein, 119 aa, synthetic construct
DIKLQQSGAELARPGASVKMSCKTSGYTFTRYHMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 424 - protein, 119 aa, synthetic construct
DIKLQQSGAELARPGASVKMSCKTSGYTETRYTMHWVKQRPGQGLEWIGYINPSRGYTNENQKFKDKATLTTDKSSSTAYMQLSSLTSEDSAVYYCARYYDDHYCLDYWGQGTTLTVSS

SEQ ID NO: 425 - protein, 119 aa, synthetic construct
DIKLQQSGAELARPGASVKMSCKTSGYTFTRYTMHWVKQRPGQGLEWIGYINPSRGYTEYNQKFKDKATLTTDKSSSTAYMQLSSLTSEDSAVYYCARYTDDHYCLDYWGQGTTLTVSS

SEQ ID NO: 426 - protein, 119 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYEDDHYCLDYWGQGTTLTVSS

SEQ ID NO: 427 - protein, 119 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARYYDDHECLDYWGQGTTLTVSS

SEQ ID NO: 428 - protein, 119 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHHCLDYWGQGTTLTVSS

SEQ ID NO: 429 - protein, 119 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCAREYDDHYCLDYWGQGTTLTVSS

SEQ ID NO: 430 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARETDDHYCLDYWGQGTTLTVSS

SEQ ID NO: 431 - protein, 119 aa, synthetic construct
DIKLQQSGAELARPGASVKMSCKTSGYTFTRYEMHWVKQRPGQGLEWIGYINPSRGYHNYNQKFKDKATLTTDKSSSTAYMQLSSLTSEDSAVYYCARYYDDHYCLDYWGQGTTLTVSS

SEQ ID NO: 432 - protein, 119 aa, synthetic construct
DIKLQQSGAELARPGASVKMSCKTSGYTFTHYEMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 433 - protein, 119 aa, synthetic construct
DIKLQQSGAELARPGASVKMSCKTSGYTFTHYTMHWVKQRPGQGLEWIGYINPSRGYTEYNQKFKDKATLTTDKSSSTAYMQLSSLTSEDSAVYYCARYYDDHYCLDYWGQGTTLTVSS

SEQ ID NO: 434 - protein, 119 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARHYDDHYCLDYWGQGTTLTVSS

SEQ ID NO: 435 - protein, 119 aa, synthetic construct
DIKLQQSGAELARPGASVKMSCKTSGYTFTRYEMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 436 - protein, 119 aa, synthetic construct
DIKLQQSGAELARPGASVKMSCKTSGYTFTRYTMHWVKQRPGQGLEWIGYINPSRGHHNYNQKFKDKATLTTDKSSSTAYMQLSSLTSEDSAVYYCARYYDDHYCLDYWGQGTTLTVSS

SEQ ID NO: 437 - protein, 119 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCAREYDDHYCLDYWGQGTTLTVSS

SEQ ID NO: 438 - protein, 119 aa, synthetic construct
QIQLVQSGAEVAKPGASVKVSCKASGYTFTRYTMHWVRQRPGQGLEWIGYINPSRGYTNYNQKFKDRATLITDKSTSTAYMELSSLTSEDTAVYYCARYYDDHYCLDYWGQGTIVIVSS

SEQ ID NO: 439 - protein, 119 aa, synthetic construct
QIQLVQSGAEVAKPGASVKVSCKASGYTFTRYTMHWVRQRPGQGLEWIGYINPSHGYTNYNQKFKDRATLITDKSTSTAYMELSSLTSEDTAVYYCARYYDDHYCLDYWGQGTIVIVSS

SEQ ID NO: 440 - protein, 106 aa, synthetic construct
DIQLTQSPAIMSASPGEKVTMTCRASSSHSYMNWYQQKSGTSPKRWIYDTSKVASGVPYRFSGSGSGTSYSLTISSMEAEDAATYYCQQWSSNPLTFGAGTKLELK

SEQ ID NO: 441 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSENPLTFGAGTKLELK

SEQ ID NO: 442 - protein, 106 aa, synthetic construct
DIQLTQSPAIMSASPGEKVTMTCRASSSVSEMNWYQQKSGTSPKRWIYDTSKVASGVPYRFSGSGSGTSYSLTISSMEAEDAATYYCQQWSSNPLTFGAGTKLELK

SEQ ID NO: 443 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCEQWSSNPLTFGAGTKLELK

SEQ ID NO: 444 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSEPLTFGAGTKLELK

SEQ ID NO: 445 - protein, 106 aa, synthetic construct
DIQLTQSPAIMSASPGEKVTMTCRASSSVSHMNWYQQKSGTSPKRWIYDTSKVASGVPYRFSGSGSGTSYSLTISSMEAEDAATYYCQQWSSNPLTFGAGTKLELK

SEQ ID NO: 446 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQESSNPLTFGAGTKLELK

SEQ ID NO: 447 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQHSSNPLTFGAGTKLELK

SEQ ID NO: 448 - protein, 106 aa, synthetic construct
DIQLTQSPAIMSASPGEKVTMTCRASSSVSYMNWYQQKSGTSPKRWIYHTSKVASGVPYRFSGSGSGTSYSLTISSMEAEDAATYYCQQWSSNPLTFGAGTKLELK

SEQ ID NO: 449 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNELTFGAGTKLELK

SEQ ID NO: 450 - protein, 5 aa, synthetic construct
RHTMH

SEQ ID NO: 451 - protein, 5 aa, synthetic construct
RYTQH

SEQ ID NO: 452 - protein, 5 aa, synthetic construct
RDTMH

SEQ ID NO: 453 - protein, 5 aa, synthetic construct
RYHMH

SEQ ID NO: 454 - protein, 5 aa, synthetic construct
RYEMH

SEQ ID NO: 455 - protein, 5 aa, synthetic construct
RYTEH

SEQ ID NO: 456 - protein, 5 aa, synthetic construct
RETMH

SEQ ID NO: 457 - protein, 17 aa, synthetic construct
YINPSRGYHNHNQKFKD

SEQ ID NO: 458 - protein, 17 aa, synthetic construct
YINPSRGYTNHNQKFHD

SEQ ID NO: 459 - protein, 17 aa, synthetic construct
YINPSRGYHHYNQHFKD

SEQ ID NO: 460 - protein, 17 aa, synthetic construct
YINPHREYTNYNQKFKD

SEQ ID NO: 461 - protein, 17 aa, synthetic construct
YINPSRGYTNYNQKHKD

SEQ ID NO: 462 - protein, 10 aa, synthetic construct
YYDDEYCLDY

SEQ ID NO: 463 - protein, 10 aa, synthetic construct
RASSSVSEMN

SEQ ID NO: 464 - protein, 10 aa, synthetic construct
RASSSVSHMN

SEQ ID NO: 465 - protein, 119 aa, synthetic construct
DIKLQQSGAELARPGASVKMSCKTSGYTFTRHTMHWVKQRPGQGLEWIGYINPSRGYTNHNQKFHDKATLTTDKSSSTAYMQLSSLTSEDSAVYYCARYYDDHYCLDYWGQGTTLTVSS

SEQ ID NO: 466 - protein, 119 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDEHYCLDYWGQGTTLTVSS

SEQ ID NO: 467 - protein, 119 aa, synthetic construct
DIKLQQSGAELARPGASVKMSCKTSGYTFTRYTMHWVKQRPGQGLEWIGYINPSRGYENYNHKFKDKATLTTDKSSSTAYMQLSSLTSEDSAVYYCARYYDDHYCLDYWGQGTTLTVSS

SEQ ID NO: 468 - protein, 119 aa, synthetic construct
DIKLQQSGAELARPGASVKMSCKTSGYTFTRYTMHWVKQRPGQGLEWIGYINPSREYTNYNQKFKDKATLTTDKSSSTAYMQLSSLTSEDSAVYYCARYYDDHYCLDYWGQGTTLTVSS

SEQ ID NO: 469 - protein, 119 aa, synthetic construct
DIKLQQSGAELARPGASVKMSCKTSGYTFTRYTMHWVKQRPGQGLEWIGYHNPSRGYTNYNQKFKDKATLTTDKSSSTAYMQLSSLTSEDSAVYYCARYYDDHYCLDYWGQGTTLTVSS

SEQ ID NO: 470 - protein, 119 aa, synthetic construct
DIKLQQSGAELARPGASVKMSCKTSGYTFTRYTMHWVKQRPGQGLEWIGYINPSRGYTHYNQKFKDKATLTTDKSSSTAYMQLSSLTSEDSAVYYCARYTDDHYCLDYWGQGTTLTVSS

SEQ ID NO: 471 - protein, 119 aa, synthetic construct
DIKLQQSGAELARPGASVKMSCKTSGYTFHRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 472 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYHDDHYCLDYWGQGTTLTVSS

SEQ ID NO: 473 - protein, 119 aa, synthetic construct
DIKLQQSGAELARPGASVKMSCKTSGYTFTRYTMHWVKQRPGQGLEWIGYIHPSRGYTNYNQKFKDKATLTTDKSSSTAYMQLSSLTSEDSAVYYCARYYDDHYCLDYWGQGTTLTVSS

SEQ ID NO: 474 - protein, 119 aa, synthetic construct
DIKLQQSGAELARPGASVKMSCKTSGYTFTRYTMHWVKQRPGQGLEWIGYINPSHGYTNYNQKFKDKATLTTDKSSSTAYMQLSSLTSEDSAVYYCARYYDDHYCLDYWGQGTTLTVSS

SEQ ID NO: 475 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARHTDDHYCLDYWGQGTTLTVSS

SEQ ID NO: 476 - protein, 119 aa, synthetic construct
DIKLQQSGAELARPGASVKMSCKTSGYTFTRYEMHWVKQRPGQGLEWIGYINPSRGYTNENQKFKDKATLTTDKSSSTAYMQLSSLTSEDSAVYYCARYYDDHYCLDYWGQGTTLTVSS

SEQ ID NO: 477 - protein, 119 aa, synthetic construct
DIKLQQSGAELARPGASVKMSCKTSGYTETRYTMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 478 - protein, 119 aa, synthetic construct
DIKLQQSGAELARPGASVKMSCKTSGYTFTRYTMHWVKQRPGQGLEWIGYINPSRGEHNYNQKFKDKATLTTDKSSSTAYMQLSSLTSEDSAVYYCARYYDDHYCLDYWGQGTTLTVSS

SEQ ID NO: 479 - protein, 119 aa, synthetic construct
DIKLQQSGAELARPGASVKMSCKTSGYTFTRYTMHWVKQRPGQGLEWIGYINPSRGETNYNQKFKDKATLTTDKSSSTAYMQLSSLTSEDSAVYYCARYTDDHYCLDYWGQGTTLTVSS

SEQ ID NO: 480 - protein, 119 aa, synthetic construct
DIKLQQSGAELARPGASVKMSCKTSGYTFTRETMHWVKQRPGQGLEWIGYINPSRGYTNYNQKFKDKATLTTDKSSSTAYMQLSSLTSEDSAVYYCARYTDDHYCLDYWGQGTTLTVSS

SEQ ID NO: 481 - protein, 119 aa, synthetic construct
DIKLQQSGAELARPGASVKMSCKTSGYTFERYTMHWVKQRPGQGLEWIGYINPSRGYHNYNQKFKDKATLTTDKSSSTAYMQLSSLTSEDSAVYYCARYYDDHYCLDYWGQGTTLTVSS

SEQ ID NO: 482 - protein, 119 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHECLDYWGQGTTLTVSS

SEQ ID NO: 483 - protein, 119 aa, synthetic construct
DIKLQQSGAELARPGASVKMSCKTSGYTFTRYTMHWVKQRPGQGLEWIGYINPSRGYHNYNQKFKDKATLTTDKSSSTAYMQLSSLTSEDSAVYYCARHYDDHYCLDYWGQGTTLTVSS

SEQ ID NO: 484 - protein, 106 aa, synthetic construct
DIQLTQSPAIMSASPGEKVTMTCRASSSVSYHNWYQQKSGTSPKRWIYDTSKVASGVPYRFSGSGSGTSYSLTISSMEAEDAATYYCQQWSSNPLTFGAGTKLELK

SEQ ID NO: 485 - protein, 106 aa, synthetic construct
DIQLTQSPAIMSASPGEKVTMTCRASSSVSYMNWYQQKSGTSPKRWIYDHSKVASGVPYRFSGSGSGTSYSLTISSMEAEDAATYYCQQWSSNPLTFGAGTKLELK

SEQ ID NO: 486 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWESNPLTFGAGTKLELK

SEQ ID NO: 487 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNHLTFGAGTKLELK

SEQ ID NO: 488 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPETFGAGTKLELK

SEQ ID NO: 489 - protein, 106 aa, synthetic construct
DIQLTQSPAIMSASPGEKVTMTCRASSSVSYMNWYQQKSGTSPKRWIYDESKVASGVPYRFSGSGSGTSYSLTISSMEAEDAATYYCQQWSSNPLTFGAGTKLELK

SEQ ID NO: 490 - protein, 106 aa, synthetic construct; identical to SEQ ID NO: 485

SEQ ID NO: 491 - protein, 106 aa, synthetic construct
DIQLTQSPAIMSASPGEKVTMTCRASSSVSYENWYQQKSGTSPKRWIYDTSKVASGVPYRFSGSGSGTSYSLTISSMEAEDAATYYCQQWSSNPLTFGAGTKLELK

SEQ ID NO: 492 - protein, 106 aa, synthetic construct; identical to SEQ ID NO: 485

SEQ ID NO: 493 - protein, 106 aa, synthetic construct
DIQLTQSPAIMSASPGEKVTMTCRASSSVEYMNWYQQKSGTSPKRWIYDTSKVASGVPYRFSGSGSGTSYSLTISSMEAEDAATYYCQQWSSNPLTFGAGTKLELK

SEQ ID NO: 494 - protein, 106 aa, synthetic construct; identical to SEQ ID NO: 484

SEQ ID NO: 495 - protein, 106 aa, synthetic construct; identical to SEQ ID NO: 487

SEQ ID NO: 496 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPHTFGAGTKLELK

SEQ ID NO: 497 - protein, 106 aa, synthetic construct
DIQLTQSPAIMSASPGEKVTMTCRASSSVSYMNWYQQKSGTSPKRWIYETSKVASGVPYRFSGSGSGTSYSLTISSMEAEDAATYYCQQWSSNPLTFGAGTKLELK

SEQ ID NO: 498 - protein, 106 aa, synthetic construct
DIQLTQSPSSLSASVGDRVTITCRASSSVSYMNWYQQKSGTAPKRWIYDTSKVASGVPYRFSGSGSGTSYTLTISSLQPEDAATYYCQQWSSNPLTFGGGTKVEIK

SEQ ID NO: 499 - protein, 106 aa, synthetic construct
DIQLTQPPSSLSASVGDRVTITCRASSSVHYMNWYQQKSGTAPKRWIYDTSKVASGVPYRFSGSGSGTSYTLTISSLQPEDAATYYCQQWSSNPLTFGGGTKVEIK

SEQ ID NO: 500 - protein, 106 aa, synthetic construct; identical to SEQ ID NO: 498

SEQ ID NO: 501 - protein, 106 aa, synthetic construct
DIQLTQSPSSLSASVGDRVTITCRASSSVHYMNWYQQKSGTAPKRWIYDTSKVASGVPYRFSGSGSGTSYTLTISSLQPEDAATYYCQQWSSNPLTFGGGTKVEIK

SEQ ID NO: 502 - protein, 5 aa, synthetic construct
RYTHH

SEQ ID NO: 503 - protein, 5 aa, synthetic construct
DYTMH

SEQ ID NO: 504 - protein, 5 aa, synthetic construct
RYTMD

SEQ ID NO: 505 - protein, 5 aa, synthetic construct
RYTMR

SEQ ID NO: 506 - protein, 5 aa, synthetic construct
HYEMH

SEQ ID NO: 507 - protein, 17 aa, synthetic construct
YINPHRGYEHYNQKFKE

SEQ ID NO: 508 - protein, 17 aa, synthetic construct
YINPHRGYTNYNQKFKD

SEQ ID NO: 509 - protein, 17 aa, synthetic construct
YINPSRGYTNYNQHFHD

SEQ ID NO: 510 - protein, 17 aa, synthetic construct
YINPSRGYENYNHKFKD

SEQ ID NO: 511 - protein, 17 aa, synthetic construct
YINPSRGYENYHQKFKH

SEQ ID NO: 512 - protein, 17 aa, synthetic construct
YINPSRGYTNYNQKFHD

SEQ ID NO: 513 - protein, 17 aa, synthetic construct
YINPSREYTNYNQKFKD

SEQ ID NO: 514 - protein, 17 aa, synthetic construct
YINPSRGYTHENQHFKE

SEQ ID NO: 515 - protein, 17 aa, synthetic construct
YINPSRGYTNYNQKFKV

SEQ ID NO: 516 - protein, 17 aa, synthetic construct
YINPSRHYTNYNQKFKD

SEQ ID NO: 517 - protein, 17 aa, synthetic construct
YHNPSRGYTNYNQKFKD

SEQ ID NO: 518 - protein, 17 aa, synthetic construct
YINPSRNYTNYNQKFKD

SEQ ID NO: 519 - protein, 17 aa, synthetic construct
YINPSRGYTNYNQHFKD

SEQ ID NO: 520 - protein, 17 aa, synthetic construct
YINPSRGYTNYNEKFKD

SEQ ID NO: 521 - protein, 17 aa, synthetic construct
YINPSRGYTNYNQKFED

SEQ ID NO: 522 - protein, 17 aa, synthetic construct
YINPSRGYHNYNQKFKV

SEQ ID NO: 523 - protein, 17 aa, synthetic construct
YINPSRGYTNYNQEFKD

SEQ ID NO: 524 - protein, 17 aa, synthetic construct
YINPSRGYHNENQKFKD

SEQ ID NO: 525 - protein, 17 aa, synthetic construct
YINPSRNYTNYNQEFKH

SEQ ID NO: 526 - protein, 17 aa, synthetic construct
YINPSRHYTNYNQKHKD

SEQ ID NO: 527 - protein, 17 aa, synthetic construct
YINPSRGYTNYNEKVKD

SEQ ID NO: 528 - protein, 17 aa, synthetic construct
YINPHRGYTNYHQKHKD

SEQ ID NO: 529 - protein, 17 aa, synthetic construct
YINPSRGYTNYNHKFKD

SEQ ID NO: 530 - protein, 17 aa, synthetic construct
YENPSRGYTNYNQKFKD

SEQ ID NO: 531 - protein, 17 aa, synthetic construct
YINPSRGYTNYNHKVKD

SEQ ID NO: 532 - protein, 17 aa, synthetic construct
YINPSRGYTNYEQKFKD

SEQ ID NO: 533 - protein, 17 aa, synthetic construct
YINPSRGYTNYNQKFKE

SEQ ID NO: 534 - protein, 17 aa, synthetic construct
YINPSRGYTNENQEFKD

SEQ ID NO: 535 - protein, 17 aa, synthetic construct
YINPSRGYTNYNQKFKH

SEQ ID NO: 536 - protein, 17 aa, synthetic construct
YINPHRGYTNYNQKFKH

SEQ ID NO: 537 - protein, 17 aa, synthetic construct
YINPSRGYTNYEHKVKD

SEQ ID NO: 538 - protein, 17 aa, synthetic construct
HINPSRGYTNYNQKFKD

SEQ ID NO: 539 - protein, 17 aa, synthetic construct
YIHPSRGYTNYNQKFKD

SEQ ID NO: 540 - protein, 17 aa, synthetic construct
YINHSRGYTNYNQKFKD

SEQ ID NO: 541 - protein, 17 aa, synthetic construct
YINPSHGYTNYNQKFKD

SEQ ID NO: 542 - protein, 17 aa, synthetic construct
EINPSRGYTNYNQKFKD

SEQ ID NO: 543 - protein, 17 aa, synthetic construct
YIEPSRGYTNYNQKFKD

SEQ ID NO: 544 - protein, 17 aa, synthetic construct
YINESRGYTNYNQKFKD

SEQ ID NO: 545 - protein, 17 aa, synthetic construct
YINPDRGYTNYNQKFKD

SEQ ID NO: 546 - protein, 17 aa, synthetic construct
YINPSEGYTNYNQKFKD

SEQ ID NO: 547 - protein, 17 aa, synthetic construct
YINPSRGDTNYNQKFKD

SEQ ID NO: 548 - protein, 17 aa, synthetic construct
YINPSRGYTDYNQKFKD

SEQ ID NO: 549 - protein, 17 aa, synthetic construct
YINPSRGYTNYNQKEKD

SEQ ID NO: 550 - protein, 17 aa, synthetic construct
YENPSRGYTNENQKFKD

SEQ ID NO: 551 - protein, 17 aa, synthetic construct
YINPHREYTNYNQKVKD

SEQ ID NO: 552 - protein, 17 aa, synthetic construct
YINPSRGYENENQKFKD

SEQ ID NO: 553 - protein, 17 aa, synthetic construct
YINPSRGYTNHNQKFKV

SEQ ID NO: 554 - protein, 17 aa, synthetic construct
YINPSRGYTNENEKHKD

SEQ ID NO: 555 - protein, 17 aa, synthetic construct
YINPSRGYTNYNQKVED

SEQ ID NO: 556 - protein, 17 aa, synthetic construct
YINPSRGYTNYEQKFKD

SEQ ID NO: 557 - protein, 17 aa, synthetic construct
YINPSRGYENYNQEFHD

SEQ ID NO: 558 - protein, 17 aa, synthetic construct
YINPSRGYTNYNQKHKH

SEQ ID NO: 559 - protein, 17 aa, synthetic construct
YHNPSRGYTNYNQKVKD

SEQ ID NO: 560 - protein, 17 aa, synthetic construct
YHNPSRGYTNYEQKFED

SEQ ID NO: 561 - protein, 17 aa, synthetic construct
YINPSRGYTNHNQKFKE

SEQ ID NO: 562 - protein, 17 aa, synthetic construct
YINPSRGYTEYNQKFKD

SEQ ID NO: 563 - protein, 17 aa, synthetic construct
YINPSRGEHNYNQKFKD

SEQ ID NO: 564 - protein, 17 aa, synthetic construct
YINPSRGETNYNQKFKD

SEQ ID NO: 565 - protein, 17 aa, synthetic construct
YINPSRGHHNYNQKFKD

SEQ ID NO: 566 - protein, 10 aa, synthetic construct
YYDEHYCLDY

SEQ ID NO: 567 - protein, 10 aa, synthetic construct
YEDDHYCLDY

SEQ ID NO: 568 - protein, 10 aa, synthetic construct
YYDHHYCLDY

SEQ ID NO: 569 - protein, 10 aa, synthetic construct
YHDDHYCLDY

SEQ ID NO: 570 - protein, 10 aa, synthetic construct
YTDHHYCLDY

SEQ ID NO: 571 - protein, 10 aa, synthetic construct
HYDDHYCLDY

SEQ ID NO: 572 - protein, 10 aa, synthetic construct
YYHDHYCLDY

SEQ ID NO: 573 - protein, 10 aa, synthetic construct
YYDDHHCLDY

SEQ ID NO: 574 - protein, 10 aa, synthetic construct
EYDDHYCLDY

SEQ ID NO: 575 - protein, 10 aa, synthetic construct
YYEDHYCLDY

SEQ ID NO: 576 - protein, 10 aa, synthetic construct
YYDDHECLDY

SEQ ID NO: 577 - protein, 10 aa, synthetic construct
YYTDHYCLDY

SEQ ID NO: 578 - protein, 10 aa, synthetic construct
NYDDHYCLDY

SEQ ID NO: 579 - protein, 10 aa, synthetic construct
YYDHEYCLDY

SEQ ID NO: 580 - protein, 10 aa, synthetic construct
ETDDHYCLDY

SEQ ID NO: 581 - protein, 10 aa, synthetic construct
HTDDHYCLDY

SEQ ID NO: 582 - protein, 10 aa, synthetic construct
RASSSHSYMN

SEQ ID NO: 583 - protein, 10 aa, synthetic construct
RASSSVSYHN

SEQ ID NO: 584 - protein, 10 aa, synthetic construct
RASSSVSYEN

SEQ ID NO: 585 - protein, 10 aa, synthetic construct
RASSSVEYMN

SEQ ID NO: 586 - protein, 7 aa, synthetic construct
DHSKVAS

SEQ ID NO: 587 - protein, 7 aa, synthetic construct
DESKVAS

SEQ ID NO: 588 - protein, 7 aa, synthetic construct
HTSKVAS

SEQ ID NO: 589 - protein, 7 aa, synthetic construct
ETSKVAS

SEQ ID NO: 590 - protein, 9 aa, synthetic construct
QQWSENPLT

SEQ ID NO: 591 - protein, 9 aa, synthetic construct
QQWESNPLT

SEQ ID NO: 592 - protein, 9 aa, synthetic construct
QQWSSNHLT

SEQ ID NO: 593 - protein, 9 aa, synthetic construct
QQWSSNPET

SEQ ID NO: 594 - protein, 9 aa, synthetic construct
EQWSSNPLT

SEQ ID NO: 595 - protein, 9 aa, synthetic construct
QQWSSEPLT

SEQ ID NO: 596 - protein, 9 aa, synthetic construct
QQESSNPLT

SEQ ID NO: 597 - protein, 9 aa, synthetic construct
QQHSSNPLT

SEQ ID NO: 598 - protein, 9 aa, synthetic construct
QQWSSNPHT

SEQ ID NO: 599 - protein, 9 aa, synthetic construct
QQWSSNELT

SEQ ID NO: 600 - protein, 121 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 601 - protein, 14 aa, synthetic construct
GGSGGSGGSGGSGG

SEQ ID NO: 602 - protein, 108 aa, synthetic construct
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 603 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = H or E
  VARIANT 10: note = N or H
  VARIANT 11: note = Y, E, or H
  VARIANT 12: note = N or H
  VARIANT 13: note = Q or H
  VARIANT 15: note = F or V
XINPXRXXXXXXXKXKD

SEQ ID NO: 604 - protein, 17 aa, synthetic construct
  VARIANT 1: note = Y or E
  VARIANT 5: note = S or R
  VARIANT 7: note = G or H
  VARIANT 8: note = Y or H
  VARIANT 9: note = T, H, or E
  VARIANT 10: note = N or H
  VARIANT 11: note = E or H
  VARIANT 12: note = N or H
  VARIANT 13: note = Q or H
  VARIANT 15: note = F or V
XINPXRXXXXXXXKXKD

SEQ ID NO: 605 - protein, 243 aa, synthetic construct
DIKLQQSGAELARPGASVKMSCKTSGYTFTHYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 606 - protein, 243 aa, synthetic construct
DIKLQQSGAELARPGASVKMSCKTSGYTFTHYTMHWVKQRPGQGLEWIGYINPSRGYTNYNHKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 607 - protein, 243 aa, synthetic construct
DIKLQQSGAELARPGASVKMSCKTSGYTFTRYTMHWVKQRPGQGLEWIGYINPSRGYTNYNQKVKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 608 - protein, 243 aa, synthetic construct
DIKLQQSGAELARPGASVKMSCKTSGYTFTRYTMHWVKQRPGQGLEWIGYINPSRGYTNENQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 609 - protein, 243 aa, synthetic construct
DIKLQQSGAELARPGASVKMSCKTSGYTHTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 610 - protein, 243 aa, synthetic construct
DIKLQQSGAELARPGASVKMSCKTSGYTFTHYTMHWVKQRPGQGLEWIGYINPSRHYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 611 - protein, 243 aa, synthetic construct
DIKLQQSGAELARPGASVKMSCKTSGYTFTRYTMHWVKQRPGQGLEWIGYINPSRGHTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 612 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 613 - protein, 243 aa, synthetic construct
DIKLQQSGAELARPGASVKMSCKTSGYTFTRYTMHWVKQRPGQGLEWIGYINPSRGYTNHNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 614 - protein, 243 aa, synthetic construct
DIKLQQSGAELARPGASVKMSCKTSGYTFTRYTMHWVKQRPGQGLEWIGYINPSRGYTNYHQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 615 - protein, 119 aa, synthetic construct
DIKLQQSGAELARPGASVKMSCKTSGYTFTHYTMHWVKQRPGQGLEWIGYINPSRGYTNYNHKFKDKATLTTDKSSSTAYMQLSSLTSEDSAVYYCARYYDDHYCLDYWGQGTTLTVSS

SEQ ID NO: 616 - protein, 119 aa, synthetic construct
DIKLQQSGAELARPGASVKMSCKTSGYTFTHYTMHWVKQRPGQGLEWIGYINPSRHYTNYNQKFKDKATLTTDKSSSTAYMQLSSLTSEDSAVYYCARYYDDHYCLDYWGQGTTLTVSS

SEQ ID NO: 617 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQEWDHNHVTFGAGTKLELK

SEQ ID NO: 618 - protein, 9 aa, synthetic construct
QEWDHNHVT

(19) United States
(12) Patent Application Publication
(10) Pub. No.: US 2025/0313630 A1
(43) Pub. Date: Oct. 9, 2025

(54) PH-SELECTIVE ANTI-CD3 ANTIBODIES AND USE OF THE SAME

(71) Applicant: BigHat Biosciences, Inc., San Mateo, CA (US)

(72) Inventors: Mark DEPRISTO, Los Altos, CA (US); Peyton GREENSIDE, San Mateo, CA (US); Ryan HENRICI, Philadelphia, PA (US)

(21) Appl. No.: 18/869,653

(22) PCT Filed: May 26, 2023
(86) PCT No.: PCT/US2023/067579

§ 371 Date: Nov. 26, 2024

Related U.S. Application Data
(60) Provisional application No. 63/346,795, filed on May 27, 2022.
(60) Provisional application No. 63/431,611, filed on Dec. 9, 2022.
(60) Provisional application No. 63/496,657, filed on Apr. 17, 2023.

(51) Int. Cl. C07K 16/28

(52) U.S. Cl. CPC C07K 16/2809; C07K 2317/31; C07K 2317/569; C07K 2317/622; C07K 2317/73; C07K 2317/92

(57) ABSTRACT

Described herein are pH-selective anti-CD3 antibodies and use of the same. The anti-CD3 antibodies provided herein exhibit substantially improved binding affinity in an acidic pH environment compared to a neutral pH environment, and do not bind or displays negligible binding to immune cells in a neutral pH environment.

### CROSS-REFERENCE

The application claims the benefit of U.S. Provisional Application No. 63/346,795, filed on May 27, 2022, U.S. Provisional Application No. 63/431,611, filed on Dec. 9, 2022, and U.S. Provisional Application No. 63/496,657, filed on Apr. 17, 2023, which are incorporated herein by reference in their entirety.

### SEQUENCE LISTING

The instant application contains a Sequence Listing which has been submitted electronically in XML format and is hereby incorporated by reference in its entirety. Said XML copy, created on Apr. 17, 2023, is named 61301_707_103_SL.xml and is 664,254 bytes in size.

### FIELD OF THE INVENTION

The present disclosure relates generally to engineered anti-CD3 antibodies that selectively bind to immune cells in an acidic tumor microenvironment.

### BACKGROUND

Antibodies therapeutics have significant promise as treatments for a multitude of hematologic and solid tissue malignancies. Bispecific T cell-engagers, for example, bind to antigens between T cells and tumor cells and thereby activate T cells to kill the tumor cells. A drawback of antibody therapies is that many tumor associated antigens are not exclusively expressed on cancer cells. Non-specific antibody therapeutics can therefore bind to tumor antigen bearing healthy cells and lead to off target toxicities.

A unique characteristic to the tumor microenvironment relative to healthy cells is its acidic pH, generally below 6.5. Therefore, if antibodies could selectively bind to antigens at acidic pH in the tumor microenvironment, this could circumvent the toxicities associated with off target binding. There remains a need to develop therapies that selectively target antigens in the tumor microenvironment as compared to healthy cells.

### SUMMARY

In certain aspects, the present disclosure provides engineered anti-hCD3e antibodies to specifically engage CD3_{ε} at weakly acidic pH, e.g., 5.8 to 6.5, and avoid or minimize binding at physiological or neutral pH, e.g. 7.0 to 7.4. In some embodiments, the engineered anti-hCD3e antibodies maintain near-wildtype binding, activation, and cytotoxicity at pH 6.0, but abrogate binding, activation, and cytotoxicity at pH 7.4.

In certain aspects, the present disclosure provides anti-CD3 antibodies or fragments thereof which selectively bind to immune cells in the tumor microenvironment. In some aspects, the disclosure provides an anti-CD3 antibody or antigen-binding fragment thereof with substantially improved binding affinity, e.g., more than 5-fold improved binding affinity, in an acidic pH environment, e.g., 5.8 to 6.5, compared to a neutral pH environment, e.g., 7.0 to 7.4. In certain embodiments, the anti-CD3 antibody or fragment does not bind or displays negligible binding to immune cells in a neutral pH environment, e.g., 7.0 to 7.4. In certain embodiments, the anti-CD3 antibody or fragment thereof has at least one amino acid modification in the variable heavy and/or variable light region, e.g., substitution, deletion or insertion, relative to an anti-CD3 wild type variable region.

In certain embodiments, the disclosure provides an anti-CD3 antibody or antigen-binding fragment thereof comprising three complementarity-determining regions (CDRs) in the heavy chain variable domain and three complementarity-determining regions (CDRs) in the light chain variable domain. In some embodiments, the disclosure provides an anti-CD3 antibody or antigen-binding fragment thereof comprising (a) a HCDR1 having at least 80% sequence identity to the amino acid sequence set out in any one of SEQ ID NOs: 47-48, (b) a HCDR2 having at least 80% sequence identity to the amino acid sequence set out in any one of SEQ ID NOs: 49-59, and (c) a HCDR3 having at least 80% sequence identity to the amino acid sequence set out in any one of SEQ ID NOs: 60-61. In certain embodiments, the disclosure provides an anti-CD3 antibody or antigen-binding fragment thereof comprising (a) a LCDR1 having at least 80% sequence identity to the amino acid sequence set out in any one of SEQ ID NOs: 62-63, (b) a LCDR2 having at least 80% sequence identity to the amino acid sequence set out in SEQ ID NO: 64, and (c) a LCDR3 having at least 80% sequence identity to the amino acid sequence set out in any one of SEQ ID NOs: 65-69 or 618. In certain embodiments, the disclosure provides an anti-CD3 antibody or antigen-binding fragment thereof comprising a heavy chain variable domain having at least 90% sequence identity to the amino acid sequence set out in any one of SEQ ID NOs: 23-37, 615 or 616. In certain embodiments, the disclosure provides an anti-CD3 antibody or antigen-binding fragment thereof comprising a light chain variable domain having at least 90% sequence identity to the amino acid sequence set out in any one of SEQ ID NOs: 39-45 or 617. In certain embodiments, the disclosure provides an anti-CD3 antibody or antigen-binding fragment thereof has at least 90% sequence identity to the amino acid sequence set out in SEQ ID NO: 1.

In certain embodiments, the disclosure provides an anti-CD3 antibody or antigen-binding fragment thereof comprising at least one amino acid mutations in the heavy chain variable domain and/or light chain variable domain. In certain embodiments, the disclosure provides anti-CD3 antibody or antigen-binding fragment thereof comprising at least one amino acid substitution at position 27-53 or 55-99 in a heavy chain variable domain as set out in SEQ ID NO: 22. In certain embodiments, anti-CD3 antibody or antigen-binding fragment thereof comprises at least one amino acid substitution at position 49-96 in a light chain variable domain as set out in SEQ ID NO: 38.

In certain embodiments, the disclosure provides an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₂INPSRX₃X₄TNX₅NQKFKD (SEQ ID NO: 77), wherein X₂ is Y or E, X₃ is G or H, X₄ is Y or H, and X₅ is Y, E, or H; and (c) a HCDR3 having an amino acid sequence YX₆DDHYCLDY (SEQ ID NO: 72), wherein X₆ is Y or T. In certain embodiments, the disclosure provides an anti-CD3 antibody or antigen-binding fragment thereof comprising: (a) a LCDR1 having an amino acid sequence RASSSVSYMN (SEQ ID NO: 62); (b) a LCDR2 having an amino acid sequence DTSKVAS (SEQ ID NO: 64); and (c) a LCDR3 having an amino acid sequence QEWSSNPLT (SEQ ID NO: 66).

In certain embodiments, the disclosure provides an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KX₁₇KD (SEQ ID NO: 74), wherein X₈ is Y or E, X₉ is S or R, X₁₀ is G or H, X₁₁ is Y or H, X₁₂ is T, H, or E, X₁₃ is N or H, X₁₄ is Y, E, or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. In certain embodiments, the disclosure provides an anti-CD3 antibody or antigen-binding fragment thereof comprising: (a) a LCDR1 having an amino acid sequence RASSSVX₁₈YMN (SEQ ID NO: 75), wherein X₁₈ is S or H; (b) a LCDR2 having an amino acid sequence DTSKVAS (SEQ ID NO: 64); and (c) a LCDR3 having an amino acid sequence X₁₉X₂₀WSSX₂₁PLT (SEQ ID NO: 88), wherein X₁₉ is Q or H, X₂₀ is Q or E, and X₂₁ is N or H

In certain aspects, the disclosure provides an anti-CD3 antibody or antigen-binding fragment thereof, wherein the anti-CD3 antibody or antigen-binding fragment thereof binding affinity for CD3 at an acidic pH is 10-fold or more greater than a binding affinity of the anti-CD3 antibody or antigen-binding fragment thereof at a non-acidic pH.

In certain aspects, the disclosure provides an anti-CD3 antibody or antigen-binding fragment thereof, wherein the anti-CD3 antibody or antigen-binding fragment thereof does not bind to CD3 at a non-acidic pH.

Also provided herein are a nucleic acid encoding the anti-CD3 antibody or antigen-binding fragment thereof described herein, an expression vector comprising the nucleic acid described herein, and a host cell comprising the nucleic acid described herein or the expression vector described herein. Further described herein is a method of producing the anti-CD3 antibody or antigen-binding fragment thereof comprising maintaining the host cell described herein and isolating or purifying the anti-CD3 antibody or antigen-binding fragment thereof produced by the host cell.

In certain aspects, the disclosure provides pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. Also provided herein is a method of treating cancer in a subject, a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Also provided herein is use of the anti-CD3 antibody or antigen-binding fragment disclosed herein, the nucleic acid disclosed herein, the expression vector disclosed herein, the host cell disclosed herein, or the pharmaceutical composition disclosed herein. Also provided herein is a kit comprising the anti-CD3 antibody or antigen-binding fragment disclosed herein, the nucleic acid disclosed herein, the expression vector disclosed herein, the host cell disclosed herein, or the pharmaceutical composition disclosed herein, and instructions for use. Also provided herein is a syringe comprising the anti-CD3 antibody or antigen-binding fragment disclosed herein, the nucleic acid disclosed herein, the expression vector disclosed herein, the host cell disclosed herein, or the pharmaceutical composition disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows amino acid sequence of variable region of the wild type anti-CD3 scFv. FIG. 1 discloses VH sequence as SEQ ID NO: 600, Linker sequence as SEQ ID NO: 601, VL sequence as SEQ ID NO: 602 and complete wild type anti-CD3 scFv sequence as SEQ ID NO: 1.

FIG. 2A-FIG. 2C shows an overview of production of the wild-type anti-CD3 scFv. FIG. 2A schematically illustrates a single-chain variable fragment (scFv) configuration. FIG. 2B shows post-purification yields for all expression of wild-type anti-CD3 scFv molecules. FIG. 2C shows post-purification CE-SDS purity results for the wild-type anti-CD3 scFv molecules. Post-purification CE-SDS purity results are expressed as percentage of sample mass (y-axis) at the dominant peak size (x-axis).

FIG. 3A-FIG. 3D shows CD3 binding and activating properties of the wild type anti-CD3 scFv. FIG. 3A depicts binding of the wild type anti-CD3 scFv with CD3^{-/-} or CD3^{+/+} isogenic Jurkat T cells at pH 7.4. FIG. 3B represents binding of serial dilution of the wild type anti-CD3 scFv to CD3^{+/+} T cells at pH 7.4 or pH 6.0. FIG. 3C schematically illustrates T cell activation assay using Jurkat(NFAT-Luc) cells and plate-adsorbed antibodies. FIG. 3D represents results of T cell activation at pH 7.4.

FIG. 4A-FIG. 4C shows a target-dependent T-cell activation by minimal bispecific antibody (bsAb) T-cell-engager (BiTE). FIG. 4A schematically illustrates a minimal head-to-tail and tail-to-head bsAb BiTE incorporating anti-CD3 scFv and anti-tumor target.

FIG. 4B represents co-culture activation experiment with serial dilution of CD3x tumor target bsAbs and 1:1 Jurkat(NFAT-Luc) cells and AsPC-1 (tumor target^{hi}) or PANC-1 (tumor target^{very lo}) cells at pH 7.4. FIG. 4C shows an activation of T cells with bsAb containing intact tumor target or dead tumor target VHHs.

FIG. 5A-FIG. 5B shows specific sequence patterns enriched in tumor infiltrating or peripheral circulating B cells receptors (BCR). FIG. 5A represents observed counts of individual BCR amino acids between intratumoral and peripheral B cells. Hydrophobic amino acids are A, F, Y, L, I, M, V, and W. Negative amino acids are E and D. Polar amino acids are N, Q, T, and S. Positive amino acids are K, H, and R. Special amino acids are G, C, and P. FIG. 5B shows frequencies of all sandwich 3-mers (e.g., XAX, XKX where A or K is the amino acid of interest and Xs are flanking amino acids, each dot is a unique 3-mer) between intratumoral and peripheral B cell repertoires.

FIG. 6A-FIG. 6B shows mutagenesis strategy and selectivity of molecules that retained binding to CD3^{+/+} Jurkat T cells. FIG. 6A shows distribution of amino acid mutations over VH sequence positions. FIG. 6B shows a selectivity index for the anti-CD3 scFv variants representing the ratio of EC₅₀ estimates determined for binding at pH 7.4 over pH 6.0.

FIG. 7A-FIG. 7F shows selective anti-CD3 scFv variants. FIG. 7A shows median selectivity index representing the ratio of EC₅₀ estimates for binding at pH 7.4 over pH 6.0. FIG. 7B and FIG. 7C represents construct mutations displayed relative to the wild type residues (top row) of the heavy chain (FIG. 7B) and the light chain (FIG. 7C). Amino acid positions are indexed with the N-terminal Asp as position zero. FIG. 7D shows binding curves for anti-CD3 variant 0224 at pH 6.0 and 7.4. FIG. 7E shows binding curves for anti-CD3 variant 0243 at pH 6.0 and 7.4. FIG. 7F shows binding curves for anti-CD3 variant 0268 at pH 6.0 and 7.4 of three representative construct mutations.

FIG. 8A-8U shows binding curves for anti-CD3 scFv wt and variants at pH 6.0 and 7.4. FIG. 8A shows binding curves for the wild type at pH 6.0 and 7.4. FIG. 8B shows binding curves for anti-CD3 scFv variant 0245 at pH 6.0 and 7.4. FIG. 8C shows binding curves for anti-CD3 scFv variant 0264 at pH 6.0 and 7.4. FIG. 8D shows binding curves for anti-CD3 scFv variant 0283 at pH 6.0 and 7.4. FIG. 8E shows binding curves for anti-CD3 scFv variant 0027 at pH 6.0 and 7.4. FIG. 8F shows binding curves for anti-CD3 scFv variant 0085 at pH 6.0 and 7.4. FIG. 8G shows binding curves for anti-CD3 scFv variant 0006 at pH 6.0 and 7.4. FIG. 8H shows binding curves for anti-CD3 scFv variant 0062 at pH 6.0 and 7.4. FIG. 8I shows binding curves for anti-CD3 scFv variant 0268 at pH 6.0 and 7.4. FIG. 8J shows binding curves for anti-CD3 scFv variant 0291 at pH 6.0 and 7.4. FIG. 8K shows binding curves for anti-CD3 scFv variant 0119 at pH 6.0 and 7.4. FIG. 8L shows binding curves for anti-CD3 scFv variant 0122 at pH 6.0 and 7.4. FIG. 8M shows binding curves for anti-CD3 scFv variant 0233 at pH 6.0 and 7.4. FIG. 8N shows binding curves for anti-CD3 scFv variant 0241 at pH 6.0 and 7.4. FIG. 8O shows binding curves for anti-CD3 scFv variant 0243 at pH 6.0 and 7.4. FIG. 8P shows binding curves for anti-CD3 scFv variant 0250 at pH 6.0 and 7.4. FIG. 8Q shows binding curves for anti-CD3 scFv variant 0026 at pH 6.0 and 7.4. FIG. 8R shows binding curves for anti-CD3 scFv variant 0094 at pH 6.0 and 7.4. FIG. 8S shows binding curves for anti-CD3 scFv variant 0224 at pH 6.0 and 7.4. FIG. 8T shows binding curves for anti-CD3 scFv variant 0261 at pH 6.0 and 7.4. FIG. 8U shows binding curves for anti-CD3 scFv variant 0280 at pH 6.0 and 7.4.

FIG. 9A-9C shows an activation of pH-engineered bsAb—T cell against pancreatic adenocarcinoma cell line in vitro. FIG. 9A-FIG. 9B shows monovalent binding of anti-CD3 antibodies reformatted as bsAb TCEs at pH 6.0 (FIG. 9A) to Jurkat T cells and at pH 7.4 (FIG. 9B). FIG. 9C shows an activation of Jurkat(NFAT-Luc) reporter cells against AsPC-1 cells in vitro at pH 7.4.

FIG. 10A-10K shows binding curves for anti-CD3 scFv variants at pH 6.0 and 7.4. FIG. 10A shows binding curves for anti-CD3 scFv variant 0434 at pH 6.0 and 7.4. FIG. 10B shows binding curves for anti-CD3 scFv variant 0435 at pH 6.0 and 7.4. FIG. 10C shows binding curves for anti-CD3 scFv variant 0436 at pH 6.0 and 7.4. FIG. 10D shows binding curves for anti-CD3 scFv variant 0437 at pH 6.0 and 7.4. FIG. 10E shows binding curves for anti-CD3 scFv variant 0438 at pH 6.0 and 7.4. FIG. 10F shows binding curves for anti-CD3 0439 variant at pH 6.0 and 7.4. FIG. 10G shows binding curves for anti-CD3 scFv variant 0441 at pH 6.0 and 7.4. FIG. 10H shows binding curves for anti-CD3 scFv variant 0442 at pH 6.0 and 7.4. FIG. 10I shows binding curves for anti-CD3 scFv variant 0443 at pH 6.0 and 7.4. FIG. 10J shows binding curves for anti-CD3 scFv variant 0305 at pH 6.0 and 7.4. FIG. 10K shows binding curves for the wild type anti-CD3 scFv at pH 6.0 and 7.4.

FIG. 11A-11K shows binding curves for anti-CD3 scFv variants at pH 6.0, 6.4, 6.6, 7.0, 7.3, and 7.6. FIG. 11A shows binding curves for the wild type anti-CD3 scFv at pH 6.0, 6.4, 6.6, 7.0, 7.3, and 7.6. FIG. 11B shows binding curves for anti-CD3 scFv variant 0006 at pH 6.0, 6.4, 6.6, 7.0, 7.3, and 7.6. FIG. 11C shows binding curves for anti-CD3 scFv variant 0062 at pH 6.0, 6.4, 6.6, 7.0, 7.3, and 7.6. FIG. 11D shows binding curves for anti-CD3 scFv variant 0440 at pH 6.0, 6.4, 6.6, 7.0, 7.3, and 7.6. FIG. 11E shows binding curves for anti-CD3 scFv variant 0441 at pH 6.0, 6.4, 6.6, 7.0, 7.3, and 7.6. FIG. 11F shows binding curves for anti-CD3 scFv variant 0439 at pH 6.0, 6.4, 6.6, 7.0, 7.3, and 7.6. FIG. 11G shows binding curves for anti-CD3 scFv variant 0434 at pH 6.0, 6.4, 6.6, 7.0, 7.3, and 7.6. FIG. 11H shows binding curves for anti-CD3 scFv variant 0305 at pH 6.0, 6.4, 6.6, 7.0, 7.3, and 7.6. FIG. 11I shows binding curves for anti-CD3 scFv variant 0280 at pH 6.0, 6.4, 6.6, 7.0, 7.3, and 7.6. FIG. 11J shows binding curves for anti-CD3 scFv variant 0119 at pH 6.0, 6.4, 6.6, 7.0, 7.3, and 7.6. FIG. 11K shows binding curves for anti-CD3 scFv variant 0438 at pH 6.0, 6.4, 6.6, 7.0, 7.3, and 7.6.

FIG. 12 shows relative T cell binding and T cell activation at pH 6.0 and 7.4 for anti-CD3 scFv wildtype and anti-CD3 scFv variants 0305, 0006, 0441, and 0438 reformatted into bispecifics with a VHH targeting a tumor antigen.

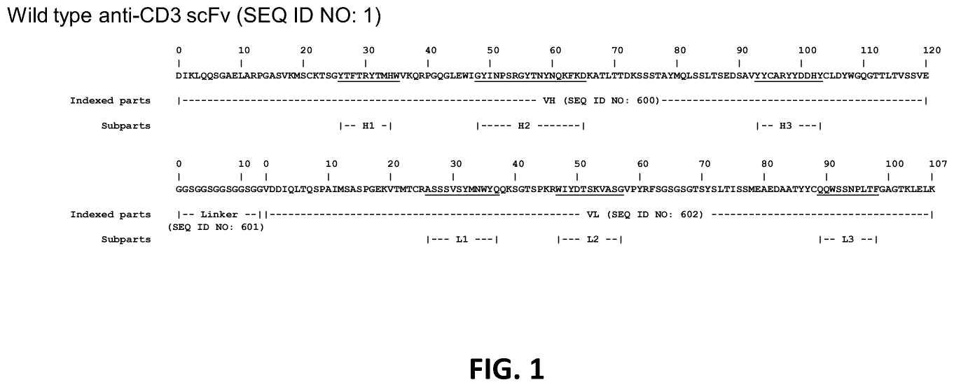

### DETAILED DESCRIPTION OF THE INVENTION

### Overview

In certain embodiments, the present disclosure provides anti-CD3 antibodies or fragments thereof which selectively bind to immune cells in the tumor microenvironment. In some aspects, the disclosure provides an anti-CD3 antibody or antigen-binding fragment thereof with substantially improved binding affinity, e.g., more than 5-fold improved binding affinity, in an acidic pH environment, e.g., 5.8 to 6.5, compared to a neutral pH environment, e.g., 7.0 to 7.4. In certain embodiments, the anti-CD3 antibody or fragment does not bind or displays negligible binding to immune cells in a neutral pH environment, e.g., 7.0 to 7.4. In certain embodiments, the anti-CD3 antibody or fragment thereof has at least one amino acid modification in the variable heavy and/or variable light region, e.g., substitution, deletion or insertion, relative to an anti-CD3 wild type variable region.

In certain embodiments, the disclosure provides an anti-CD3 antibody or antigen-binding fragment thereof comprising (a) a HCDR1 having the amino acid sequence set out in any one of SEQ ID NOs: 47-48, (b) a HCDR2 having at least 80% sequence identity to the amino acid sequence set out in any one of SEQ ID NOs: 49-59, and (c) a HCDR3 having at least 80% sequence identity to the amino acid sequence set out in any one of SEQ ID NOs: 60-61. Further provided herein is an anti-CD3 antibody or antigen-binding fragment thereof that comprises at least one of (a) a HCDR1 comprising the amino acid sequence set out in any one of SEQ ID NOs: 47-48, (b) a HCDR2 comprising the amino acid sequence set out in any one of SEQ ID NOs: 49-59, or (c) a HCDR3 comprising the amino acid sequence set out in any one of SEQ ID NOs: 60-61. In some embodiments, the anti-CD3 antibody or antigen-binding fragment thereof comprises at least one of (a) a LCDR1 comprising the amino acid sequence set out in any one of SEQ ID NOs: 62-63, (b) a LCDR2 comprising the amino acid sequence set out in SEQ ID NO: 64, or (c) a LCDR3 comprising the amino acid sequence set out in any one of SEQ ID NOs: 65-69 or 618. In some embodiments, the anti-CD3 antibody or antigen-binding fragment thereof comprises a heavy chain variable domain having at least 90% sequence identity to the amino acid sequence set out in any one of SEQ ID NOs: 23-37, 615 or 616. In some embodiments, the anti-CD3 antibody or antigen-binding fragment thereof comprises a heavy chain variable domain having the amino acid sequence set out in any one of SEQ ID NOs: 23-37, 615 or 616. In some embodiments, the anti-CD3 antibody or antigen-binding fragment thereof comprises a light chain variable domain having at least 90% sequence identity to the amino acid sequence set out in any one of SEQ ID NOs: 39-45 or 617. In some embodiments, the anti-CD3 antibody or antigen-binding fragment thereof comprises a light chain variable domain having the amino acid sequence set out in any one of SEQ ID NOs: 39-45 or 617. In some embodiments, the anti-CD3 antibody or antigen-binding fragment thereof has at least 90% sequence identity to the amino acid sequence set out in SEQ ID NO: 1. In some embodiments, the anti-CD3 antibody or antigen-binding fragment thereof has at least 95% sequence identity to the amino acid sequence set out in SEQ ID NO: 1 In some embodiments, the anti-CD3 antibody or antigen-binding fragment thereof has at least 96% sequence identity to the amino acid sequence set out in SEQ ID NO: 1. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, a nucleic acid encoding the anti-CD3 antibody or antigen binding fragment thereof is described herein. In some embodiments, the nucleic acid comprises DNA, RNA, ssRNA, siRNA, microRNA, or mRNA. Provided herein are expression vectors comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein is for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein are kits comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising (a) a HCDR1 having at least 80% sequence identity to the amino acid sequence set out in any one of SEQ ID NOs: 47-48, (b) a HCDR2 having the amino acid sequence set out in any one of SEQ ID NOs: 49-59, and (c) a HCDR3 having at least 80% sequence identity to the amino acid sequence set out in any one of SEQ ID NOs: 60-61. Further provided herein is an anti-CD3 antibody or antigen-binding fragment thereof described herein that comprises at least one of (a) a HCDR1 comprising the amino acid sequence set out in any one of SEQ ID NOs: 47-48, (b) a HCDR2 comprising the amino acid sequence set out in any one of SEQ ID NOs: 49-59, or (c) a HCDR3 comprising the amino acid sequence set out in any one of SEQ ID NOs: 60-61. In some embodiments, the anti-CD3 antibody or antigen-binding fragment thereof comprises at least one of (a) a LCDR1 comprising the amino acid sequence set out in any one of SEQ ID NOs: 62-63, (b) a LCDR2 comprising the amino acid sequence set out in SEQ ID NO: 64, or (c) a LCDR3 comprising the amino acid sequence set out in any one of SEQ ID NOs: 65-69 or 618. In some embodiments, the anti-CD3 antibody or antigen-binding fragment thereof comprises a heavy chain variable domain having at least 90% sequence identity to the amino acid sequence set out in any one of SEQ ID NOs: 23-37, 615 or 616. In some embodiments, the anti-CD3 antibody or antigen-binding fragment thereof comprises a heavy chain variable domain having the amino acid sequence set out in any one of SEQ ID NOs: 23-37, 615 or 616. In some embodiments, the anti-CD3 antibody or antigen-binding fragment thereof comprises a light chain variable domain having at least 90% sequence identity to the amino acid sequence set out in any one of SEQ ID NOs: 39-45 or 617. In some embodiments, the anti-CD3 antibody or antigen-binding fragment thereof comprises a light chain variable domain having the amino acid sequence set out in any one of SEQ ID NOs: 39-45 or 617. In some embodiments, the anti-CD3 antibody or antigen-binding fragment thereof has at least 90% sequence identity to the amino acid sequence set out in SEQ ID NO: 1. In some embodiments, the anti-CD3 antibody or antigen-binding fragment thereof has at least 95% sequence identity to the amino acid sequence set out in SEQ ID NO: 1. In some embodiments, the anti-CD3 antibody or antigen-binding fragment thereof has at least 96% sequence identity to the amino acid sequence set out in SEQ ID NO: 1. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is a nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein is for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising (a) a HCDR1 having at least 80% sequence identity to the amino acid sequence set out in any one of SEQ ID NOs: 47-48, (b) a HCDR2 having at least 80% sequence identity to the amino acid sequence set out in any one of SEQ ID NOs: 49-59, and (c) a HCDR3 having the amino acid sequence set out in any one of SEQ ID NOs: 60-61. Further provided herein is an anti-CD3 antibody or antigen-binding fragment thereof described herein that comprises at least one of (a) a HCDR1 comprising the amino acid sequence set out in any one of SEQ ID NOs: 47-48, (b) a HCDR2 comprising the amino acid sequence set out in any one of SEQ ID NOs: 49-59, or (c) a HCDR3 comprising the amino acid sequence set out in any one of SEQ ID NOs: 60-61. In some embodiments, the anti-CD3 antibody or antigen-binding fragment thereof comprises at least one of (a) a LCDR1 comprising the amino acid sequence set out in any one of SEQ ID NOs: 62-63, (b) a LCDR2 comprising the amino acid sequence set out in SEQ ID NO: 64, or (c) a LCDR3 comprising the amino acid sequence set out in any one of SEQ ID NOs: 65-69 or 618. In some embodiments, the anti-CD3 antibody or antigen-binding fragment thereof comprises a heavy chain variable domain having at least 90% sequence identity to the amino acid sequence set out in any one of SEQ ID NOs: 23-37, 615 or 616. In some embodiments, the anti-CD3 antibody or antigen-binding fragment thereof comprises a heavy chain variable domain having the amino acid sequence set out in any one of SEQ ID NOs: 23-37, 615 or 616. In some embodiments, the anti-CD3 antibody or antigen-binding fragment thereof comprises a light chain variable domain having at least 90% sequence identity to the amino acid sequence set out in any one of SEQ ID NOs: 39-45 or 617. In some embodiments, the anti-CD3 antibody or antigen-binding fragment thereof comprises a light chain variable domain having the amino acid sequence set out in any one of SEQ ID NOs: 39-45 or 617. In some embodiments, the anti-CD3 antibody or antigen-binding fragment thereof has at least 90% sequence identity to the amino acid sequence set out in SEQ ID NO: 1. In some embodiments, the anti-CD3 antibody or antigen-binding fragment thereof has at least 95% sequence identity to the amino acid sequence set out in SEQ ID NO: 1. In some embodiments, the anti-CD3 antibody or antigen-binding fragment thereof has at least 96% sequence identity to the amino acid sequence set out in SEQ ID NO: 1. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising (a) a LCDR1 having the amino acid sequence set out in any one of SEQ ID NOs: 62-63, (b) a LCDR2 having at least 80% sequence identity to the amino acid sequence set out in SEQ ID NO: 64, and (c) a LCDR3 having at least 80% sequence identity to the amino acid sequence set out in any one of SEQ ID NOs: 65-69 or 618. Further provided herein is an anti-CD3 antibody or antigen-binding fragment thereof described herein comprising at least one of (a) a HCDR1 comprising the amino acid sequence set out in any one of SEQ ID NOs: 47-48, (b) a HCDR2 comprising the amino acid sequence set out in any one of SEQ ID NOs: 49-59, or (c) a HCDR3 comprising the amino acid sequence set out in any one of SEQ ID NOs: 60-61. In some embodiments, the anti-CD3 antibody or antigen-binding fragment thereof comprises at least one of (a) a LCDR1 comprising the amino acid sequence set out in any one of SEQ ID NOs: 62-63, (b) a LCDR2 comprising the amino acid sequence set out in SEQ ID NO: 64, or (c) a LCDR3 comprising the amino acid sequence set out in any one of SEQ ID NOs: 65-69 or 618. In some embodiments, the anti-CD3 antibody or antigen-binding fragment thereof comprises a heavy chain variable domain having at least 90% sequence identity to the amino acid sequence set out in any one of SEQ ID NOs: 23-37, 615 or 616. In some embodiments, the anti-CD3 antibody or antigen-binding fragment thereof comprises a heavy chain variable domain having the amino acid sequence set out in any one of SEQ ID NOs: 23-37, 615 or 616. In some embodiments, the anti-CD3 antibody or antigen-binding fragment thereof comprises a light chain variable domain having at least 90% sequence identity to the amino acid sequence set out in any one of SEQ ID NOs: 39-45 or 617. In some embodiments, the anti-CD3 antibody or antigen-binding fragment thereof comprises a light chain variable domain having the amino acid sequence set out in any one of SEQ ID NOs: 39-45 or 617. In some embodiments, the anti-CD3 antibody or antigen-binding fragment thereof has at least 90% sequence identity to the amino acid sequence set out in SEQ ID NO: 1. In some embodiments, the anti-CD3 antibody or antigen-binding fragment thereof has at least 95% sequence identity to the amino acid sequence set out in SEQ ID NO: 1. In some embodiments, the anti-CD3 antibody or antigen-binding fragment thereof has at least 96% sequence identity to the amino acid sequence set out in SEQ ID NO: 1. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein is for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising (a) a LCDR1 having at least 80% sequence identity to the amino acid sequence set out in any one of SEQ ID NOs: 62-63, (b) a LCDR2 having at least 80% sequence identity to the amino acid sequence set out in SEQ ID NO: 64, and (c) a LCDR3 having the amino acid sequence set out in any one of SEQ ID NOs: 65-69 or 618. Further provided herein is an anti-CD3 antibody or antigen-binding fragment thereof described herein comprising at least one of (a) a HCDR1 comprising the amino acid sequence set out in any one of SEQ ID NOs: 47-48, (b) a HCDR2 comprising the amino acid sequence set out in any one of SEQ ID NOs: 49-59, or (c) a HCDR3 comprising the amino acid sequence set out in any one of SEQ ID NOs: 60-61. In some embodiments, the anti-CD3 antibody or antigen-binding fragment thereof comprises at least one of (a) a LCDR1 comprising the amino acid sequence set out in any one of SEQ ID NOs: 62-63, (b) a LCDR2 comprising the amino acid sequence set out in SEQ ID NO: 64, or (c) a LCDR3 comprising the amino acid sequence set out in any one of SEQ ID NOs: 65-69 or 618. In some embodiments, the anti-CD3 antibody or antigen-binding fragment thereof comprises a heavy chain variable domain having at least 90% sequence identity to the amino acid sequence set out in any one of SEQ ID NOs: 23-37, 615 or 616. In some embodiments, the anti-CD3 antibody or antigen-binding fragment thereof comprises a heavy chain variable domain having the amino acid sequence set out in any one of SEQ ID NOs: 23-37, 615 or 616. In some embodiments, the anti-CD3 antibody or antigen-binding fragment thereof comprises a light chain variable domain having at least 90% sequence identity to the amino acid sequence set out in any one of SEQ ID NOs: 39-45 or 617. In some embodiments, the anti-CD3 antibody or antigen-binding fragment thereof comprises a light chain variable domain having the amino acid sequence set out in any one of SEQ ID NOs: 39-45 or 617. In some embodiments, the anti-CD3 antibody or antigen-binding fragment thereof has at least 90% sequence identity to the amino acid sequence set out in SEQ ID NO: 1. In some embodiments, the anti-CD3 antibody or antigen-binding fragment thereof has at least 95% sequence identity to the amino acid sequence set out in SEQ ID NO: 1. In some embodiments, the anti-CD3 antibody or antigen-binding fragment thereof has at least 96% sequence identity to the amino acid sequence set out in SEQ ID NO: 1. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiment, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 50, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 65. In some embodiments, the anti-CD3 antibody or antigen-binding fragment thereof comprises a heavy chain variable domain having at least 80% sequence identity to the amino acid sequence set out in SEQ ID NO: 23. In some embodiments, the anti-CD3 antibody or antigen-binding fragment thereof comprises a light chain variable domain having at least 80% sequence identity to the amino acid sequence set out in SEQ ID NO: 39. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 49, a HCDR3 as set out in SEQ ID NO: 61, a LCDR1 as set out in SEQ ID NO: 62, a LCDR2 as set out in SEQ ID NO: 64, and/or a LCDR3 as set out in SEQ ID NO: 66. In some embodiments, the anti-CD3 antibody or antigen-binding fragment thereof of claim 18, wherein the anti-CD3 antibody or antigen-binding fragment thereof comprises a heavy chain variable domain having at least 80% sequence identity to the amino acid sequence set out in SEQ ID NO: 24. In some embodiments, the anti-CD3 antibody or antigen-binding fragment thereof comprises a light chain variable domain having at least 80% sequence identity to the amino acid sequence set out in SEQ ID NO: 40. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 65. In some embodiments, the anti-CD3 antibody or antigen-binding fragment thereof comprises a heavy chain variable domain having at least 80% sequence identity to the amino acid sequence set out in SEQ ID NO: 25. In some embodiments, the anti-CD3 antibody or antigen-binding fragment thereof comprises a light chain variable domain having at least 80% sequence identity to the amino acid sequence set out in SEQ ID NO: 41. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 51, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 65. In some embodiments, the anti-CD3 antibody or antigen-binding fragment thereof comprises a heavy chain variable domain having at least 80% sequence identity to the amino acid sequence set out in SEQ ID NO: 26. In some embodiments, the anti-CD3 antibody or antigen-binding fragment thereof comprises a light chain variable domain having at least 80% sequence identity to the amino acid sequence set out in SEQ ID NO: 39. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 65. In some embodiments, the anti-CD3 antibody or antigen-binding fragment thereof comprises a heavy chain variable domain having at least 80% sequence identity to the amino acid sequence set out in SEQ ID NO: 27. In some embodiments the anti-CD3 antibody or antigen-binding fragment thereof comprises a light chain variable domain having at least 80% sequence identity to the amino acid sequence set out in SEQ ID NO: 39. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 25. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 529, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 615. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 54, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 359. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 51, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 31. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 318. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 48, a HCDR2 as set out in SEQ ID NO: 516, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 616. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 50, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 368. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiment, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 22. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 53, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 322. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising a HCDR1 as set out in SEQ ID NO: 47, a HCDR2 as set out in SEQ ID NO: 55, a HCDR3 as set out in SEQ ID NO: 60, a LCDR1 as set out in SEQ ID NO: 62, a LCDR2 as set out in SEQ ID NO: 64, and/or a LCDR3 as set out in SEQ ID NO: 618. In some embodiments, the anti-CD3 antibody or antigen-binding fragment thereof comprises a heavy chain variable domain having at least 80% sequence identity to the amino acid sequence set out in SEQ ID NO: 316. In some embodiments the anti-CD3 antibody or antigen-binding fragment thereof comprises a light chain variable domain having at least 80% sequence identity to the amino acid sequence set out in SEQ ID NO: 617. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising at least one amino acid substitution at position 27-53 or 55-99 in a heavy chain variable domain as set out in SEQ ID NO: 22. In some embodiments, the at least one amino acid substitution at position 27-53 or 55-59 in heavy chain variable domain comprises at least two, three, or four amino acid substitutions. In some embodiments, the at least one amino acid substitution at position 27-53 or 55-59 in heavy chain variable domain comprises a substitution of an amino acid with H, E, T, D, G, or V. In some embodiments, the at least one amino acid substitution at position 27-53 or 55-59 in heavy chain variable domain comprises T27G, T27E, F28H, R30H, Y31D, Y31H, I50E, I50H, S53H, G55H, T57E, T57H, N58H, Y59H, Y59E, N60H, Q61H, K62H, K63V, K64H, D65E, D65H, or Y99T. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising at least one amino acid substitution at position 49-96 in a light chain variable domain as set out in SEQ ID NO: 38. In some embodiments, the at least one amino acid substitution at position 49-96 in a light chain variable domain comprises a substitution of an amino acid with H, D, V or E. In some embodiments, the at least one amino acid substitution at position 49-96 in a light chain variable domain comprises any one or more amino acid substitutions comprising Y49E, Q89H, Q90E, S92D, S93H, N94H, P95H, L96V, or L96H. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence HYTMH (SEQ ID NO: 48); (b) a HCDR2 having an amino acid sequence X₂INPSRX₃X₄TNX₅NQKFKD (SEQ ID NO: 71), wherein X₂ is Y or E, X₃ is G or H, X₄ is Y or H, and X₅ is Y, E, or H; and (c) a HCDR3 having an amino acid sequence YX₆DDHYCLDY (SEQ ID NO: 72), wherein X₆ is Y or T. Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence EINPSRX₃X₄TNX₅NQKFKD (SEQ ID NO: 77), wherein X₃ is G or H, X₄ is Y or H, and X₅ is Y, E, or H; and (c) a HCDR3 having an amino acid sequence YX₆DDHYCLDY (SEQ ID NO: 72), wherein X₆ is Y or T. Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₂INPSRHX₄TNX₅NQKFKD (SEQ ID NO: 78), wherein X₂ is Y or E, X₄ is Y or H, and X₅ is Y, E, or H; and (c) a HCDR3 having an amino acid sequence YX₆DDHYCLDY (SEQ ID NO: 72), wherein X₆ is Y or T. Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₂INPSRX₃HTNX₅NQKFKD (SEQ ID NO: 79), wherein X₂ is Y or E, X₃ is G or H, and X₅ is Y, E, or H; and (c) a HCDR3 having an amino acid sequence YX₆DDHYCLDY (SEQ ID NO: 72), wherein X₆ is Y or T. Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₂INPSRX₃X₄TNX₅NQKFKD (SEQ ID NO: 71), wherein X₂ is Y or E, X₃ is G or H, X₄ is Y or H, and X₅ is E, or H; and (c) a HCDR3 having an amino acid sequence YX₆DDHYCLDY (SEQ ID NO: 72), wherein X₆ is Y or T. Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₂INPSRX₃X₄TNX₅NQKFKD (SEQ ID NO: 71), wherein X₂ is Y or E, X₃ is G or H, X₄ is Y or H, X₅ is Y, E, or H; and (c) a HCDR3 having an amino acid sequence YTDDHYCLDY (SEQ ID NO: 61). Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a LCDR1 having an amino acid sequence RASSSVSYMN (SEQ ID NO: 62); (b) a LCDR2 having an amino acid sequence DTSKVAS (SEQ ID NO: 64); and (c) a LCDR3 having an amino acid sequence QEWSSNPLT (SEQ ID NO: 66). Further provided herein is an anti-CD3 antibody or antigen-binding fragment thereof described herein comprising one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence HYTMH (SEQ ID NO: 48); (b) a HCDR2 having an amino acid sequence X₈NPX₉RX₁₀X₁₁X₁₂X₁₃X₁₄X₁₅X₁₆KX₁₇KD (SEQ ID NO: 74), wherein X₈ is Y or E, X₉ is S or R, X₁₀ is G or H, X₁₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence EINPX₉RX₁₀X₁₁X₁₂X₁₃X₁₄X₁₅X₁₆KX₁₇KD (SEQ ID NO: 80), X₉ is S or R, X₁₀ is G or H, X₁₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPRRX₁₀X₁₁X₁₂X₁₃X₁₄X₁₅X₁₆KX₁₇KD (SEQ ID NO: 81), wherein X₈ is Y or E, X₁₀ is G or H, X₁₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RHX₁₁X₁₂X₁₃X₁₄X₁₅X₁₆KX₁₇KD (SEQ ID NO: 82), wherein X₈ is Y or E, X₉ is S or R, X₁₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀HX₁₂X₁₃X₁₄X₁₅X₁₆KX₁₇KD (SEQ ID NO: 83), wherein X₈ is Y or E, X₉ is S or R, X₁₀ is G or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provided herein is an anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KX₁₇KD (SEQ ID NO: 603), wherein X₈ is Y or E, X₉ is S or R, X₁₀ is G or H, X₁ is Y or H, X₁₂ is H or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄), (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂HX₁₄X₁₅X₁₆KX₁₇KD (SEQ ID NO: 84), wherein X₈ is Y or E, X₉ is S or R, X₁₀ is G or H, X₁ is Y or H, X₁₂ is T, H, or E, X₁₄ is Y, E, or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KX₁₇KD (SEQ ID NO: 604), wherein X₈ is Y or E, X₉ is S or R, X₁₀ is G or H, X₁₁ is Y or H, X₁₂ is T, H, or E, X₁₃ is N or H, X₁₄ is E or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄HX₁₆KX₁₇KD (SEQ ID NO: 85), wherein X₈ is Y or E, X₉ is S or R, X₁₀ is G or H, X₁ is Y or H, X₁₂ is T, H, or E, X₁₃ is N or H, X₁₄ is Y, E, or H, X₁₆ is Q or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅HKX₁₇KD (SEQ ID NO: 86), wherein X₈ is Y or E, X₉ is S or R, X₁₀ is G or H, X₁₁ is Y or H, X₁₂ is T, H, or E, X₁₃ is N or H, X₁₄ is Y, E, or H, X₅ is N or H, and X₁₇ is F or V; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KVKD (SEQ ID NO: 87), wherein X₈ is Y or E, X₉ is S or R, X₁₀ is G or H, X₁₁ is Y or H, X₁₂ is T, H, or E, X₁₃ is N or H, X₁₄ is Y, E, or H, X₁₅ is N or H, and X₁₆ is Q or H; and (c) a HCDR3 having an amino acid sequence YX₆DDHYCLDY (SEQ ID NO: 72), wherein X₆ is Y or T. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₅X₁₆KX₁₇KD (SEQ ID NO: 74), wherein X₈ is Y or E, X₉ is S or R, X₁₀ is G or H, X₁₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YTDDHYCLDY (SEQ ID NO: 61). Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a LCDR1 having an amino acid sequence RASSSVX₁₈YMN (SEQ ID NO: 75), wherein X₁₈ is S or H; (b) a LCDR2 having an amino acid sequence DTSKVAS (SEQ ID NO: 64); and (c) a LCDR3 having an amino acid sequence HX₂₀WSSX₂₁PLT (SEQ ID NO: 88), wherein X₂₀ is Q or E, and X₂₁ is N or H. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a LCDR1 having an amino acid sequence RASSSVX₁₈YMN (SEQ ID NO: 75), wherein X₁₈ is S or H; (b) a LCDR2 having an amino acid sequence DTSKVAS (SEQ ID NO: 64); and (c) a LCDR3 having an amino acid sequence X₁₉EWSSX₂₁PLT (SEQ ID NO: 89), wherein X₁₉ is Q or H, and X₂₁ is N or H. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof comprising: (a) a LCDR1 having an amino acid sequence RASSSVX₁₈YMN (SEQ ID NO: 75), wherein X₁₈ is S or H; (b) a LCDR2 having an amino acid sequence DTSKVAS (SEQ ID NO: 64); and (c) a LCDR3 having an amino acid sequence X₁₉X₂₀WSSHPLT (SEQ ID NO: 90), wherein X₁₉ is Q or H, and X₂₀ is Q or E. Further provide herein is the anti-CD3 antibody or antigen-binding fragment thereof, wherein the anti-CD3 antibody or antigen-binding fragment thereof comprises one or more amino acid mutations selected from F28H, T29 W, Q89H, and Q89E in a heavy chain variable domain as set out in SEQ ID NO: 22. In some embodiments, the anti-CD3 antibody or antigen-binding fragment thereof comprises a Y49E mutation in a light chain variable domain as set out in SEQ ID NO: 38. In some embodiments, the anti-CD3 antibody or antigen-binding fragment thereof disclosed herein comprises a linker peptide. In some embodiments, the linker peptide comprises about 2-20 amino acids. In some embodiments, the linker peptide comprises (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n is selected from 1 to 10. In some embodiments, the linker peptide comprises VEGGSGGSGGSGGSGGVD (SEQ ID NO: 46). In some embodiments, the linker peptide links a portion of a heavy chain variable domain to a portion of a light chain variable domain. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof, wherein the anti-CD3 antibody or antigen-binding fragment thereof binding affinity for CD3 at an acidic pH is 10-fold or more greater than a binding affinity of the anti-CD3 antibody or antigen-binding fragment thereof to CD3 at a non-acidic pH. In some embodiments, the anti-CD3 antibody or antigen-binding fragment thereof comprises a sequence disclosed herein. In some embodiments, the anti-CD3 antibody or antigen-binding fragment thereof binding affinity for CD3 at an acidic pH is 20-fold or more greater than a binding affinity of the anti-CD3 antibody or antigen-binding fragment thereof to CD3 at a non-acidic pH. In some embodiments, the anti-CD3 antibody or antigen-binding fragment thereof binding affinity for CD3 at an acidic pH is 30-fold or more greater than a binding affinity of the anti-CD3 antibody or antigen-binding fragment thereof to CD3 at a non-acidic pH. In some embodiments, an acidic pH comprises a pH of 6.0. In some embodiments, a non-acidic pH comprises a pH of 7.4. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

Provided herein is an anti-CD3 antibody or antigen-binding fragment thereof, wherein the anti-CD3 antibody or antigen-binding fragment thereof does not bind to CD3 at a non-acidic pH. In some embodiments, the anti-CD3 antibody or antigen-binding fragment thereof comprises a sequence disclosed herein. In some embodiments, the anti-CD3 antibody or antigen-binding fragment thereof has an EC₅₀ for binding CD3 of more than 1.0×10⁻⁷ M at a non-acidic pH. In some embodiments, the anti-CD3 antibody or antigen-binding fragment thereof has an EC₅₀ for binding CD3 of more than 3.0×10⁻⁷ M at a non-acidic pH. In some embodiments, the anti-CD3 antibody or antigen-binding fragment thereof has an EC₅₀ for binding CD3 of more than 5.0×10⁻⁷ M at a non-acidic pH. In some embodiments, an acidic pH comprises a pH of 6.0. In some embodiments, a non-acidic pH comprises a pH of 7.4. In some embodiments, the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof. In some embodiments, the anti-CD3 antibody or antigen-binding fragment thereof is a scFv antibody. In some embodiments, the anti-CD3 antibody or antigen-binding fragment thereof is a VHH antibody. In some embodiments, the anti-CD3 antibody or antigen-binding fragment thereof is humanized. In some embodiments, the anti-CD3 antibody or antigen-binding fragment thereof is nonhuman antibody. In some embodiments, the anti-CD3 antibody or antigen-binding fragment thereof is a human antibody. In some embodiments, disclosed herein is a nucleic acid encoding the anti-CD3 antibody or antigen binding fragment thereof described herein. In some embodiments, the nucleic acid comprises DNA, RNA, ssRNA, siRNA, microRNA, or mRNA. Provided herein is an expression vector comprising the nucleic acid described herein. Provided herein is a host cell comprising the nucleic acid described herein or the expression vector described herein. Provided herein is a method of producing the anti-CD3 antibody or antigen-binding fragment thereof described herein, the method comprising maintaining the host cell described herein in a medium to produce the anti-CD3 antibody or antigen-binding fragment thereof and isolating or purifying the anti-CD3 antibody or antigen-binding fragment thereof produced by the host cell. Provided herein is a pharmaceutical composition for treating cancer, comprising the anti-CD3 antibody or antigen-binding fragment thereof described herein, the nucleic acid disclosed herein, the expression vector disclosed herein or the host cell disclosed herein and a pharmaceutically acceptable carrier or excipient. In some embodiments, the pharmaceutical composition further comprises at least one additional therapeutic agent. In some embodiments, the nucleic acid disclosed herein, the expression vector of disclosed herein, the host cell disclosed herein, the method disclosed herein or the pharmaceutical composition disclosed herein for use in a therapy. In some embodiments, the nucleic acid disclosed herein, the expression vector disclosed herein, the host cell disclosed herein, the method disclosed herein, or the pharmaceutical composition disclosed herein is for use in a therapy, wherein the therapy is an antibody monotherapy, antibody-drug conjugate (ADC) therapy, T cell-engaging immunotherapy, or CAR-T therapy. Provided herein is a method for treating cancer in a subject in need thereof comprising administering to the subject a therapeutically effective amount of the anti-CD3 antibody or antigen-binding fragment disclosed herein, the nucleic acid disclosed herein, the expression vector disclosed herein, the host cell disclosed herein, or the pharmaceutical composition disclosed herein. Provided herein is a use of the anti-CD3 antibody or antigen-binding fragment thereof disclosed herein, in the manufacture of a medicament for treatment of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer is neuroendocrine, colon adenocarcinoma, rectal adenocarcinoma, pancreatic adenocarcinoma, gastric carcinoma, or esophageal carcinoma. In some embodiments, the cancer comprises a solid tumor. In some embodiments, the solid tumor comprises adenocarcinoma, glioblastoma, colorectal carcinoma, neuroblastoma, or osteosarcoma. Provided herein is a use of the anti-CD3 antibody or antigen-binding fragment thereof disclosed herein. In some embodiments, the use is for diagnosis of cancer. In some embodiments, the cancer is pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer. In some embodiments, the cancer comprises a solid tumor. In some embodiments, the solid tumor comprises adenocarcinoma, glioblastoma, colorectal carcinoma, neuroblastoma, or osteosarcoma. Provided herein is a kit comprising the anti-CD3 antibody or antigen-binding fragment thereof disclosed herein, the nucleic acid disclosed herein, the expression vector disclosed herein, the host cell disclosed herein, or the pharmaceutical composition disclosed herein, and instructions for use. Provided herein is a syringe comprising the anti-CD3 antibody or antigen-binding fragment thereof disclosed herein, the nucleic acid disclosed herein, the expression vector disclosed herein, the host cell disclosed herein, or the pharmaceutical composition disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “a” and “an” refers to one or more (i.e., at least one) of the grammatical object of the article. By way of example, “a polypeptide” encompasses one or more polypeptides.

As used herein, the terms “about” and “approximately,” when used to modify an amount specified in a numeric value or range, indicate that the numeric value as well as reasonable deviations from the value known to the skilled person in the art, for example±20%, +10%, or +5%, are within the intended meaning of the recited value.

Unless otherwise specified, a “nucleotide sequence encoding a protein” includes all nucleotide sequences that are degenerate versions of each other and thus encode the same amino acid sequence.

The term “subject” is intended to include any mammal. In some embodiments, the subject is a cat, a dog, a goat, a human, a non-human primate, a rodent (e.g., a mouse or a rat), a pig, or a sheep.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “antigen-binding domain” is used to refer to one or more antibody variable domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

Unless otherwise specified, a “nucleotide sequence encoding a protein” includes all nucleotide sequences that are degenerate versions of each other and thus encode the same amino acid sequence.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

The term “exogenous” refers to any material introduced from or originating from outside a cell, a tissue, or an organism that is not produced by or does not originate from the same cell, tissue, or organism in which it is being introduced.

The term “antibody” refers to a protein with an immunoglobulin fold that specifically binds to an antigen via its variable region or regions. The term “antibody” is used herein in the broadest sense and encompasses monoclonal antibodies, polyclonal antibodies, dimers, multimers, multispecific antibodies (e.g., bispecific antibodies), and antibody fragments, so long as they exhibit the desired biological activity (Miller et al., J Immunol. 170:4854-4861, 2003). Antibodies may be murine, human, humanized, chimeric, or derived from other species. (Janeway, C., Travers, P., Walport, M., Shlomchik (2001) Immunol. Biology, 5th Ed., Garland Publishing, New York). A target antigen generally has numerous binding sites, also called epitopes, recognized by CDRs (complementarity determining regions) on multiple antibodies. Each antibody that specifically binds to a different epitope has a different structure. Thus, one antigen may have more than one corresponding antibody. An antibody can, e.g., include a full-length immunoglobulin molecule or an immunologically active portion of a full-length immunoglobulin molecule, i.e., a molecule that contains an antigen-binding site that immunospecifically binds an antigen of a target of interest or part thereof. The immunoglobulin disclosed herein can be of any type (e.g., IgG, IgE, IgM, IgD, and IgA), class (e.g., IgG₁, IgG₂, IgG₃, IgG₄, IgA₁ and IgA₂) or subclass of immunoglobulin molecule. The immunoglobulins can be derived from any species. In one aspect, however, the immunoglobulin is of human, murine, or rabbit origin.

Antibodies bound to various types of molecules, such as polyethylene glycols (PEGs), may be used as modified antibodies. Methods for modifying antibodies are already established in the art.

The term “antibody fragments” refers to a portion of a full-length antibody or a polypeptide that includes a portion of a full-length antibody, that retains antigen-binding activity via its variable region or regions. Examples of antibody fragments include Fab, Fab′, F(ab′)₂, and Fv fragments; diabodies; linear antibodies; minibodies (Olafsen et al., Protein Eng. Design & Sel. 17(4):315-323, 2004), fragments produced by a Fab expression library, single-chain antibody molecules; and multispecific antibodies formed from antibody fragments.

The term “complementarity determining region” or “CDR” refers to one of the three hypervariable regions (or HVRs) that are known to confer (at least in part) antigen-binding specificity in each antibody light chain and each antibody heavy. The three CDRs in the antibody heavy chain and the antibody light chain interrupt four framework regions in the heavy chain variable domain and the light chain variable domain. The CDRs of each chain are typically referred to as CDR1, CDR2, and CDR3, numbered sequentially starting from the N-terminus, and are also typically identified by the chain in which the particular CDR is located.

The “framework regions” or “FRs” of different light immunoglobulin chains and different heavy immunoglobulin chains are relatively conserved within different antibodies produced by a mammal. The framework regions of light and heavy immunoglobulin chains serve to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

“Monospecific” antigen-binding protein refers to the ability of the antigen-binding protein, such as an antibody, to bind only one epitope. “Bispecific” antigen-binding protein refers to the ability of the antigen-binding protein to bind two different epitopes. “Multispecific” antigen-binding protein refers to the ability of the antigen-binding protein to bind more than one epitope. In certain embodiments, a multispecific antigen-binding protein, such as a multispecific antibody, encompasses a bispecific antigen-binding protein or a bispecific antibody. For bispecific and multispecific antigen-binding proteins provided herein, the epitopes can be on the same antigen, or each epitope can be on a different antigen. Therefore, in certain embodiments, a multispecific antigen-binding protein provided herein, such as a bispecific antibody, binds to two different antigens. In certain embodiments, the multispecific antigen-binding protein, such as a bispecific antibody, binds to different epitopes on one antigen. In certain embodiments, a multispecific antigen-binding protein provided herein binds to each epitope with a dissociation constant (Kd) of about <1 M, about <100 nM, about <10 nM, about <1 nM, about <0.1 nM, about <0.01 nM, or about <0.001 nM (e.g., about 10⁻⁸ M or less, e.g., from about 10⁻⁸ M to about 10⁻¹³ M, e.g., from about 10⁻⁹ M to about 10⁻¹⁰ M).

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val).

The term “administer” refers to a method of polypeptides or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. In one embodiment, the compositions described herein are administered intravenously.

The term “transduced”, “transfected”, or “transformed” refers to a process by which an exogenous nucleic acid is introduced or transferred into a cell. A “transduced”, “transfected,” or “transformed” cell (e.g., mammalian cell) is one that has been transduced, transfected, or transformed with exogenous nucleic acid (e.g., a vector) that includes an exogenous nucleic acid encoding any of the polypeptides described herein.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (e.g., asparagine and glutamine), aliphatic family (e.g., alanine, valine, leucine and isoleucine), aromatic family (e.g., phenylalanine, tryptophan, and tyrosine).

The term “promoter” means a DNA sequence recognized by enzymes/proteins in a cell (e.g., a mammalian cell, a hepatocyte) required to initiate the transcription of an operably linked coding sequence (e.g., a nucleic acid encoding a polypeptide (e.g., any of the exemplary polypeptides described herein). A promoter typically refers, to e.g. a nucleotide sequence to which an RNA polymerase and/or any associated factor binds and at which transcription is initiated. The promoter can be constitutive, inducible, or tissue-specific (e.g., a liver-specific promoter).

The term “enhancer” refers to a nucleotide sequence that can increase the transcription of an operably linked nucleic acid (e.g., a nucleic acid encoding a polypeptide (e.g., any of the exemplary polypeptides described herein). An enhancer can increase the level of transcription by providing additional binding sites for transcription-associated proteins (e.g., transcription factors). Unlike promoters, enhancers can act at distances further away from the transcription start site (e.g., as compared to a promoter).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 or Megalign. The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The term “affinity” refers to the strength of the sum of all non-covalent interactions between an antigen-binding site and its antigen. Unless otherwise indicated, “affinity” refers to intrinsic binding affinity, which reflects a 1:1 interaction between an antigen-binding domain and an antigen. Affinity can be measured, e.g., using surface plasmon resonance (SPR) technology (e.g., BIACORE®) or biolayer interferometry (e.g., FORTEBIO®). Additional methods for determining the affinity of an antigen-binding domain and its antigen are known in the art.

The term “single-chain polypeptide” means a polypeptide comprising a single polypeptide chain.

The term “multi-chain polypeptide” means a complex of two or more (e.g., 2, 3, 4, 5, 6, 7, or 8) polypeptide chains (e.g., the same or different polypeptide chains) that covalently and/or non-covalently associate with each other. For example, two or more polypeptide chains of a multi-chain polypeptide can associate through the use of two domains that associate with each other (e.g., two Fc domains or IL-15 and the sushi domain of IL-15 receptor alpha).

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. The Kabat numbering of residues may be determined for a given antibody by alignment at regions of homology of the sequence of the antibody with a “standard” Kabat numbered sequence. It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342:877-883.

Other numbering conventions for CDR sequences available to a skilled person include “AbM” (University of Bath) and “contact” (University College London) methods. The minimum overlapping region using at least two of the Kabat, Chothia, AbM, and contact methods can be determined to provide the ‘minimum binding unit’. The minimum binding unit may be a sub-portion of a CDR. Table I below represents one definition using each numbering convention for each CDR or binding unit. The Kabat numbering scheme is used in Table 1 to number variable domain amino acid sequence. It should be noted that some of the CDR definition may vary depending on the individual publication used.

TABLE 1

| | | Chothia | | | Minimum |
|---|---|---|---|---|---|
| | Kabat CDR | CDR | AbM CDR | Contact CDR | Binding Unit |
| H1 | 31-35/35A/35B | 26-32/33/34 | 26-35/35A/35B | 30-35/35A/35B | 31-32 |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

The section headings used herein are for organizational purposes only and are not to be construed as limiting the subject matter described.

### CDRS Modification & Lead Sequences

The anti-CD3 antibody or antigen-binding fragment thereof may have at least one or more amino acid modifications in various regions of the antibody. The anti-CD3 antibody or antigen-binding fragment thereof may be modified by at least one or more amino acid substitution, deletion, addition, or a combination thereof at one or more position while the variant CD3 binding protein substantially retains the biological characteristics of the unmodified protein, such as binding to CD3. In some embodiments, the amino acid sequence of the CD3 antibody or antigen-binding fragment thereof may have at least 1, at least 2, at least 3, at least 4, at least 5, at least 6, at least 7, at least 8, at least 9, at least 10, at least 11, at least 12, at least 13, at least 14, or at least 15 amino acid insertions, deletions, substitutions or a combination thereof.

In some instances, one or more amino acid modifications described herein can be in the antigen-binding (Fab) fragments of an antibody or antibody binding fragment thereof. In some instances, the modification can be within the Fab region of a heavy chain polypeptide (H) and a first light chain polypeptide (L). In some embodiments, the modification can be within a heavy chain variable domain (VH), a heavy chain constant domain (CHI), alight chain variable domain (VL), a light chain constant domain (CL), or a combination thereof. In some embodiments, one or more amino acid modification can be in the Fab VH and VL with the N-terminal region. The VH, VL, HC or LC sequence disclosed herein may be a variant sequence with up to 15 amino acid substitutions, insertion, or deletions. For example, the variant sequence may have up to 15, 14, 13, 12, 11, 10, 9, 8, 7, 6, 5, 4, 3, 2, or 1 amino acid substitution(s), addition(s), or deletion(s). The sequence variation may exclude one or more or all of the CDRs, for example, the CDRs are the same as the VH, VL, HC or LC sequence and the variation is in the remaining portion of the VH or VL, HC, or LC sequence, so that the CDR sequences are fixed and intact. In some embodiments, the anti-CD3 antibody or antigen-binding fragment thereof can have 60, 70, 80, 90, 95, 100% identity to at least about 5, 10, 15, 20, 25, 30, 35, 40, 45, 50, 60, 70, 90, 100, consecutive amino acid of a HCDR, LCDR, light chain variable region, heavy chain variable region, light chain, heavy chain, constant region or a frame work region disclosed herein.

Each of HCDR1, HCDR2, HCDR3, LCDR1, LCDR2, and LCDR3 may be modified alone or in combination with any other CDR, in any permutation or combination. Typically, the modification is a substitution, particularly a conserved substitution, for example in Table 2 below.

TABLE 2

| Side Chain | Members |
|---|---|
| Hydrophobic | Met, Ala, Val, Leu Ile |
| Neutral Hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

In some embodiments, the anti-CD3 antibody or an antigen-binding fragment thereof may comprise CDRs: HCDR1 selected from the group consisting of SEQ ID NOs: 47-48, HCDR2 selected from the group consisting of SEQ ID NOs: 49-50, HCDR3 selected from the group consisting of SEQ ID NOs: 60-61, LCDR1 selected from the group consisting of SEQ ID NOs: 62-63, LCDR2 of SEQ ID NO: 64, and LCDR3 selected from the group consisting of SEQ ID NOs: 65-69 and 618, functional fragments thereof, functional variants thereof, and combination thereof.

In some embodiments, the anti-CD3 antibody or an antigen-binding fragment thereof may comprise CDRs: HCDR1 of SEQ ID NO: 47, HCDR2 of SEQ ID NO: 50, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 51, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof.

In some embodiments, the anti-CD3 antibody or an antigen-binding fragment thereof may comprise CDRs: HCDR1 of SEQ ID NO: 48, HCDR2 of SEQ ID NO: 50, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 48, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 48, HCDR2 of SEQ ID NO: 51, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 48, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 65, functional fragments thereof, functional variants thereof, and combination thereof. In some embodiments, the anti-CD3 antibody or an antigen-binding fragment thereof may comprise CDRs: HCDR1 of SEQ ID NO: 48, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 48, HCDR2 of SEQ ID NO: 529, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 54, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 51, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 48, HCDR2 of SEQ ID NO: 516, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 50, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 49, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 53, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof. In some embodiments, the anti-CD3 antibody or an antigen-binding fragment thereof may comprise CDRs: HCDR1 of SEQ ID NO: 47, HCDR2 of SEQ ID NO: 55, HCDR3 of SEQ ID NO: 60, LCDR1 of SEQ ID NO: 62, LCDR2 of SEQ ID NO: 64, and LCDR3 of SEQ ID NO: 618, functional fragments thereof, functional variants thereof, and combination thereof.

In some embodiments, each domain of three complementarity-determining region LCDR: LCDR1, LCDR2, and LCDR3 for VL, and HCDR-1, HCDR-2, and HCDR-3 for VH may be modified alone or in combination with any other CDR, in any permutation or combination. In some embodiments, the LCDR-1, LCDR-2, LCDR-3, HCDR-1, HCDR2, and HCDR-3 may be modified by substitution, deletion or insertion of 1 amino acid to 10 amino acids. In some embodiments, the LCDR-1, LCDR-2, LCDR-3, HCDR-1, HCDR2, and HCDR-3 may have 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 1 amino acid to 9 amino acids, 1 amino acid to 10 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 2 amino acids to 9 amino acids, 2 amino acids to 10 amino acids, 3 amino acids to 4 amino acids, 3 amino to 5 amino acids, 3 amino acids to 6 amino acids, 3 amino acids to 7 amino acids, 3 amino acids to 8 amino acids, 3 amino acids to 9 amino acids, 3 amino acids to 10 amino acids, 4 amino acids to 5 amino acids, 4 amino acids to 6 amino acids, 4 amino acids to 7 amino acids, 4 amino acids to 8 amino acids, 4 amino acids to 9 amino acids, 4 amino acids to 10 amino acids, 5 amino acids to 6 amino acids, 5 amino acids to 7 amino acids, 5 amino acids to 8 amino acids, 5 amino acids to 9 amino acids, 5 amino acids to 10 amino acids, 6 amino acids to 7 amino acids, 6 amino acids to 8 amino acids, 6 amino acids to 9 amino acids, 6 amino acids to 10 amino acids, 7 amino acids to 8 amino acids, 7 amino acids to 9 amino acids, 7 amino acids to 10 amino acids, 8 amino acids to 9 amino acids, 8 amino acids to 10 amino acids, or 9 amino acids to 10 amino acids modifications. In some embodiments, the LCDR-1, LCDR-2, LCDR-3, HCDR-1, HCDR2, and HCDR-3 may have 1 amino acid, 2 amino acids, 3 amino acids, 4 amino acids, 5 amino acids, 6 amino acids, 7 amino acids, 8 amino acids, 9 amino acids, or 10 amino acids modifications. In some embodiments, the LCDR-1, LCDR-2, LCDR-3, HCDR-1, HCDR2, and HCDR-3 may have at least 1 amino acid, 2 amino acids, 3 amino acids, 4 amino acids, 5 amino acids, 6 amino acids, 7 amino acids, 8 amino acids, or 9 amino acids modifications. In some embodiments, the LCDR-1, LCDR-2, LCDR-3, HCDR-1, HCDR2, and HCDR-3 may have at most 2 amino acids, 3 amino acids, 4 amino acids, 5 amino acids, 6 amino acids, 7 amino acids, 8 amino acids, 9 amino acids, or 10 amino acids modifications.

In some embodiments, the anti-CD3 antibodies or antigen-binding thereof may comprise HCDR1 selected from the group consisting of SEQ ID NOs: 47-48 or a variant HCDR1 thereof. In some embodiments, the HCDR1 may include a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 47-48.

In some embodiments, the anti-CD3 antibodies or antigen-binding thereof may comprise HCDR2 selected from the group consisting of SEQ ID NOs: 49-50 or a variant HCDR2 thereof. In some embodiments, the polypeptide may comprise a HCDR2 having the amino acid sequence set out in any one of SEQ ID NOs: 49-50. In some embodiments, the HCDR2 includes a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 49-50.

In some embodiments, the anti-CD3 antibodies or antigen-binding thereof may comprise HCDR3 selected from the group consisting of SEQ ID NOs: 60-61 or a variant HCDR3 thereof. In some embodiments, the anti-CD3 antibody or antigen-binding fragment thereof may include a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 60-61.

In some embodiments, the anti-CD3 antibody or an antigen-binding fragment thereof may comprise HCDR1 having an amino acid sequence of X₁YTMH (SEQ ID NO: 70), wherein the X₁ may be selected from: R or H.

In some embodiments, the anti-CD3 antibody or an antigen-binding fragment thereof may comprise HCDR2 having an amino acid sequence of X₂INPSRX₃X₄TNX₅NQKFKD (SEQ ID NO: 71), wherein:
X₂ may be selected from: Y or E;
X₃ may be selected from: G or H;
X₄ may be selected from: Y or H; or
X₅ is selected from: Y, E, or H.

In some embodiments, the anti-CD3 antibody or an antigen-binding fragment thereof may comprise HCDR2 having an amino acid sequence of X₈INPX₉RX₁₀X₁₁X₁₂X₁₃X₁₄X₁₅X₁₆KX₁₇KD (SEQ ID NO: 74), wherein:
X₈ may be selected from: Y or E;
X₉ may be selected from: S or R;
X₁₀ may be selected from: G or H;
X₁₁ may be selected from: Y or H;
X₁₂ may be selected from: T, H, or E;
X₁₃ may be selected from: N or H;
X₁₄ may be selected from: Y, E, or H;
X₁₅ may be selected from: N or H;
X₁₆ may be selected from: Q or H; or
X₁₇ may be selected from: F or V.

In some embodiments, the anti-CD3 antibody or an antigen-binding fragment thereof may comprise HCDR3 having an amino acid sequence of YX₆DDHYCLDY (SEQ ID NO: 72), wherein X₆ may be selected from: Y, or T.

In some embodiments, the anti-CD3 antibodies or antigen-binding thereof may comprise LCDR1 selected from the group consisting of SEQ ID NOs: 62-63 or a variant LCDR1 thereof. In some embodiments, the LCDR1 includes a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 62-63.

In some embodiments, the anti-CD3 antibodies or antigen-binding thereof may comprise LCDR2 selected from the group consisting of SEQ ID NO: 64 or a variant LCDR2 thereof. In some embodiments, the LCDR2 includes a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NO: 64.

In some embodiments, the anti-CD3 antibodies or antigen-binding thereof may comprise LCDR3 selected from the group consisting of SEQ ID NO: 65-69 and 618 or a variant LCDR3 thereof. In some embodiments, the anti-CD3 antibody or antigen-binding fragment thereof may comprise LCDR3 comprising a sequence selected from a group consisting of SEQ ID NOS: 65-69 and 618. In some embodiments, the LCDR3 includes a sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 65-69 or 618.

In some embodiments, the anti-CD3 antibody or an antigen-binding fragment thereof may comprise LCDR1 having an amino acid sequence of RASSSVX₁₈YMN (SEQ ID NO: 75), wherein X₁₈ may be S or H.

In some embodiments, the anti-CD3 antibody or an antigen-binding fragment thereof may comprise LCDR2 having an amino acid sequence of DTSKVAS (SEQ ID NO: 64).

In some embodiments, the anti-CD3 antibody or an antigen-binding fragment thereof may comprise LCDR3 having an amino acid sequence of a X₁₉X₂₀WSSX₂₁PLT (SEQ ID NO: 76), wherein
X₁₉ may be Q or H;
X₂₀ may be Q or E; or
X₂₁ may be N or H.

In some embodiments, the anti-CD3 antibodies or antigen-binding fragment thereof may comprise a heavy chain variable region selected from the group consisting of SEQ ID NOs: 23-37, 615 and 616, functional fragments thereof, and functional variants thereof. In some embodiments, the anti-CD3 antibodies or antigen-binding thereof may comprise a light chain variable region selected from the group consisting of SEQ ID NOs: 39-45 and 617, functional fragments thereof, and functional variants thereof.

In some embodiments, the anti-CD3 antibodies or antigen-binding fragment thereof may comprise a heavy chain variable region of SEQ ID NO: 23 and alight chain variable region of SEQ ID NO: 39. In some embodiments, the anti-CD3 antibodies or antigen-binding fragment thereof may comprise a heavy chain variable region of SEQ ID NO: 24 and a light chain variable region of SEQ ID NO: 40. In some embodiments, the anti-CD3 antibodies or antigen-binding fragment thereof may comprise a heavy chain variable region of SEQ ID NO: 25 and a light chain variable region of SEQ ID NO: 41. In some embodiments, the anti-CD3 antibodies or antigen-binding fragment thereof may comprise a heavy chain variable region of SEQ ID NO: 26 and a light chain variable region of SEQ ID NO: 39. In some embodiments, the anti-CD3 antibodies or antigen-binding fragment thereof may comprise a heavy chain variable region of SEQ ID NO: 27 and a light chain variable region of SEQ ID NO: 39. In some embodiments, the anti-CD3 antibodies or antigen-binding fragment thereof may comprise a heavy chain variable region of SEQ ID NO: 25 and a light chain variable region of SEQ ID NO: 617. In some embodiments, the anti-CD3 antibodies or antigen-binding fragment thereof may comprise a heavy chain variable region of SEQ ID NO: 615 and a light chain variable region of SEQ ID NO: 617. In some embodiments, the anti-CD3 antibodies or antigen-binding fragment thereof may comprise a heavy chain variable region of SEQ ID NO: 359 and a light chain variable region of SEQ ID NO: 617. In some embodiments, the anti-CD3 antibodies or antigen-binding fragment thereof may comprise a heavy chain variable region of SEQ ID NO: 31 and a light chain variable region of SEQ ID NO: 617. In some embodiments, the anti-CD3 antibodies or antigen-binding fragment thereof may comprise a heavy chain variable region of SEQ ID NO: 318 and a light chain variable region of SEQ ID NO: 617. In some embodiments, the anti-CD3 antibodies or antigen-binding fragment thereof may comprise a heavy chain variable region of SEQ ID NO: 616 and a light chain variable region of SEQ ID NO: 617. In some embodiments, the anti-CD3 antibodies or antigen-binding fragment thereof may comprise a heavy chain variable region of SEQ ID NO: 368 and a light chain variable region of SEQ ID NO: 617. In some embodiments, the anti-CD3 antibodies or antigen-binding fragment thereof may comprise a heavy chain variable region of SEQ ID NO: 22 and a light chain variable region of SEQ ID NO: 617. In some embodiments, the anti-CD3 antibodies or antigen-binding fragment thereof may comprise a heavy chain variable region of SEQ ID NO: 322 and a light chain variable region of SEQ ID NO: 617. In some embodiments, the anti-CD3 antibodies or antigen-binding fragment thereof may comprise a heavy chain variable region of SEQ ID NO: 316 and a light chain variable region of SEQ ID NO: 617.

In some embodiments, a heavy chain variable domain and/or a light chain variable domain described herein may be modified by at least one amino acid substitution, deletion or insertion, wherein the variant anti-CD3 antibodies or antigen-binding fragment thereof substantially retains the biological characteristics of the unmodified protein, such as binding to CD3. In some embodiments, a heavy chain variable domain and/or a light chain variable domain may be modified alone or in combination with any other CDR, in any permutation or combination. In some embodiments, the heavy chain variable domain and/or light chain variable domain may be modified by the substitution, deletion or insertion of 1 amino acid to 10 amino acids. In some embodiments, the heavy chain variable domain and/or light chain variable domain may have 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 1 amino acid to 9 amino acids, 1 amino acid to 10 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 2 amino acids to 9 amino acids, 2 amino acids to 10 amino acids, 3 amino acids to 4 amino acids, 3 amino to 5 amino acids, 3 amino acids to 6 amino acids, 3 amino acids to 7 amino acids, 3 amino acids to 8 amino acids, 3 amino acids to 9 amino acids, 3 amino acids to 10 amino acids, 4 amino acids to 5 amino acids, 4 amino acids to 6 amino acids, 4 amino acids to 7 amino acids, 4 amino acids to 8 amino acids, 4 amino acids to 9 amino acids, 4 amino acids to 10 amino acids, 5 amino acids to 6 amino acids, 5 amino acids to 7 amino acids, 5 amino acids to 8 amino acids, 5 amino acids to 9 amino acids, 5 amino acids to 10 amino acids, 6 amino acids to 7 amino acids, 6 amino acids to 8 amino acids, 6 amino acids to 9 amino acids, 6 amino acids to 10 amino acids, 7 amino acids to 8 amino acids, 7 amino acids to 9 amino acids, 7 amino acids to 10 amino acids, 8 amino acids to 9 amino acids, 8 amino acids to 10 amino acids, or 9 amino acids to 10 amino acids modifications. In some embodiments, the LCDR and/or HCDR may have 1 amino acid, 2 amino acids, 3 amino acids, 4 amino acids, 5 amino acids, 6 amino acids, 7 amino acids, 8 amino acids, 9 amino acids, or 10 amino acids modifications. In some embodiments, the heavy chain variable domain and/or light chain variable domain may have at least 1 amino acid, 2 amino acids, 3 amino acids, 4 amino acids, 5 amino acids, 6 amino acids, 7 amino acids, 8 amino acids, or 9 amino acids modification. In some embodiments, the heavy chain variable domain and/or light chain variable domain may have at most 2 amino acids, 3 amino acids, 4 amino acids, 5 amino acids, 6 amino acids, 7 amino acids, 8 amino acids, 9 amino acids, or 10 amino acids modification.

In some embodiments, the anti-CD3 antibodies or antigen-binding thereof may comprise a heavy chain variable domain selected from the group consisting of SEQ ID NOs: 23-37, 615 and 616 or a variant heavy chain variable domain thereof. In some embodiments, the anti-CD3 antibody or antigen-binding fragment thereof may include a HCDR sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 23-37, 615 or 616.

In some embodiments, the anti-CD3 antibodies or antigen-binding thereof may comprise a light chain variable domain selected from the group consisting of SEQ ID NOs: 39-45 and 617 or a variant light chain variable domain thereof. In some embodiments, the anti-CD3 antibody or antigen-binding fragment thereof may include a light chain variable domain sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 39-45 or 617.

In some embodiments, the anti-CD3 antibody or antigen-binding fragment thereof may comprise a LCDR1 having an amino acid sequence RASSSVSYMN (SEQ ID NO: 62). In some embodiments, In some embodiments, the anti-CD3 antibody or antigen-binding fragment thereof may comprise a LCDR2 having an amino acid sequence DTSKVAS (SEQ ID NO: 64). In some embodiments, the anti-CD3 antibody or antigen-binding fragment thereof may comprise a LCDR3 having an amino acid sequence QEWSSNPLT (SEQ ID NO: 66). In some embodiments, the anti-CD3 antibody or antigen-binding fragment thereof may comprise a LCDR1 having an amino acid sequence RASSSVSYMN (SEQ ID NO: 62), a LCDR2 having an amino acid sequence DTSKVAS (SEQ ID NO: 64), a LCDR3 having an amino acid sequence QEWSSNPLT (SEQ ID NO: 66) or any combination thereof.

In some embodiments, the anti-CD3 antibodies or antigen-binding thereof can comprise SEQ ID NO: 1. The anti-CD3 antibodies or antigen-binding thereof described herein may be modified by at least one amino acid substitution, deletion or insertion, wherein the variant anti-CD3 antibodies or antigen-binding fragment thereof substantially retains the biological characteristics of the unmodified protein, such as binding to CD3. In some embodiments, the anti-CD3 antibodies or antigen-binding thereof may be modified by the substitution, deletion or insertion of 1 amino acid to 10 amino acids. In some embodiments, the anti-CD3 antibodies or antigen-binding thereof may have 1 amino acid to 2 amino acids, 1 amino acid to 3 amino acids, 1 amino acid to 4 amino acids, 1 amino acid to 5 amino acids, 1 amino acid to 6 amino acids, 1 amino acid to 7 amino acids, 1 amino acid to 8 amino acids, 1 amino acid to 9 amino acids, 1 amino acid to 10 amino acids, 2 amino acids to 3 amino acids, 2 amino acids to 4 amino acids, 2 amino acids to 5 amino acids, 2 amino acids to 6 amino acids, 2 amino acids to 7 amino acids, 2 amino acids to 8 amino acids, 2 amino acids to 9 amino acids, 2 amino acids to 10 amino acids, 3 amino acids to 4 amino acids, 3 amino to 5 amino acids, 3 amino acids to 6 amino acids, 3 amino acids to 7 amino acids, 3 amino acids to 8 amino acids, 3 amino acids to 9 amino acids, 3 amino acids to 10 amino acids, 4 amino acids to 5 amino acids, 4 amino acids to 6 amino acids, 4 amino acids to 7 amino acids, 4 amino acids to 8 amino acids, 4 amino acids to 9 amino acids, 4 amino acids to 10 amino acids, 5 amino acids to 6 amino acids, 5 amino acids to 7 amino acids, 5 amino acids to 8 amino acids, 5 amino acids to 9 amino acids, 5 amino acids to 10 amino acids, 6 amino acids to 7 amino acids, 6 amino acids to 8 amino acids, 6 amino acids to 9 amino acids, 6 amino acids to 10 amino acids, 7 amino acids to 8 amino acids, 7 amino acids to 9 amino acids, 7 amino acids to 10 amino acids, 8 amino acids to 9 amino acids, 8 amino acids to 10 amino acids, or 9 amino acids to 10 amino acids modifications. In some embodiments, the anti-CD3 antibodies or antigen-binding thereof may have 1 amino acid, 2 amino acids, 3 amino acids, 4 amino acids, 5 amino acids, 6 amino acids, 7 amino acids, 8 amino acids, 9 amino acids, or 10 amino acids modifications. In some embodiments, the anti-CD3 antibodies or antigen-binding thereof may have at least 1 amino acid, 2 amino acids, 3 amino acids, 4 amino acids, 5 amino acids, 6 amino acids, 7 amino acids, 8 amino acids, or 9 amino acids modification. In some embodiments, the anti-CD3 antibodies or antigen-binding thereof may have at most 2 amino acids, 3 amino acids, 4 amino acids, 5 amino acids, 6 amino acids, 7 amino acids, 8 amino acids, 9 amino acids, or 10 amino acids modification.

In some embodiments, the anti-CD3 antibodies or antigen-binding fragment thereof as disclosed herein can comprise an amino acid sequence, and the amino acid sequence of the polypeptide may be at least 60%, 65%, 70%, 75%, 80%, 85%, 90%, 91%, 92%, 93%, 94%, 95%, 96%, 97%, 98%, 99%, or 100% sequence identity to SEQ ID NO: 1. For example, the amino acid sequence of the anti-CD3 antibodies or antigen-binding fragment thereof can be between 80% and about 100% identical to the polypeptide sequence of SEQ ID NO: 1.

### Amino Acid Substitution

The HCDR1, HCDR2, HCDR3, LCDR1, LCDR2, and LCDR3 can exhibit one of a finite number of main chain conformations (canonicals). The particular canonical structure class of a CDR can be defined by both the length of the CDR and by the loop packing, determined by residues located at key position in both the CDRs and the framework regions. One or more changes in canonical structures by adding, deleting, substituting one or more amino acid within the key position may result in distinct changes in the antibody (e.g., antigen sensitivity or binding affinity). For example, introduction of one or more histidine residues in the binding interface can be used to engineer pH sensitivity of antibody binding. While histidine residues in the binding interface mediate pH sensitivity through their electrostatic interactions introducing of ionizable residues in the protein core may also lead to pH-dependent conformational changes, and therefore, pH sensitivity of binding.

In some embodiments, the anti-CD3 antibodies or antigen-binding thereof may comprise at least one, at least two, at least three, at least four, at least five, at least six, at least seven, at least eight, at least nine, at least ten amino acid substitutions at position 27-53 or 55-99 (according to Kabat numbering) in a heavy chain variable domain of SEQ ID NO: 22. In some cases, the substitution of amino acid may be with H, E, T, D, W, G or V. In some embodiments, the substitution at position at position 27-53 or 55-59 (according to Kabat numbering) in heavy chain variable domain may comprise T27G, T27E, F28H, R30H, Y31D, Y31H, 150E, I50H, S53H, G₅₅H, T57E, T57H, N58H, Y59H, Y59E, N60H, Q61H, K62H, K63V, K64H, D65E, D65H, Y99T or any combination thereof. In some embodiments, the anti-CD3 antibody or antigen-binding fragment thereof may comprise one or more amino acid mutations selected from F28H, T29 W, Q89H, and Q89E in a heavy chain variable domain of SEQ ID NO: 22.

In some embodiments, the anti-CD3 antibodies or antigen-binding thereof may comprise at least one, at least two, at least three, at least four, at least five, at least six, at least seven, at least eight, at least nine, at least ten amino acid substitutions at position 49-96 (according to Kabat numbering) in a light chain variable domain of SEQ ID NO: 38. In some cases, the substitution of amino acid may be with H, D, V or E. In some embodiments, the substitution at position at position 49-96 (according to Kabat numbering) in heavy chain variable domain may comprise Y49E, Q89H, Q90E, S92D, S93H, N94H, P95H, L96V or L96H (according to Kabat numbering). In some embodiments, the anti-CD3 antibody or antigen-binding fragment thereof may comprise a Y49E mutation in a light chain variable domain as set out in SEQ ID NO: 38.

### pH Sensitivity (Antibody Properties)

Despite the exquisite specificity and high affinity of antibody-based cancer therapies, treatment effectiveness and binding affinity of the engineered antibodies can be hindered by a low pH tumor microenvironment. The anti-CD3 antibody or antigen-binding fragment thereof described herein can have reduced on-target binding affinity at pH 7.4, while preserving or increasing binding affinity at pH 6.0, which allow a strong gradient of activity between pH 7.4 and pH 6.0 to drive intratumoral cytokine release, promoting the expansion of effector infiltrating immune cells and recruiting additional effector cells from circulation.

In some embodiments, the binding affinity of the anti-CD3 antibody or antigen-binding fragment thereof to CD3 at acidic pH may be about 2 fold to about 1000 fold greater than a binding affinity of the anti-CD3 antibody or antigen-binding fragment thereof to CD3 at a non-acidic pH. In some embodiments, the binding affinity of the anti-CD3 antibody or antigen-binding fragment thereof to CD3 at acidic pH may be about 2 fold to about 4 fold, about 2 fold to about 6 fold, about 2 fold to about 8 fold, about 2 fold to about 10 fold, about 2 fold to about 15 fold, about 2 fold to about 20 fold, about 2 fold to about 25 fold, about 2 fold to about 30 fold, about 2 fold to about 35 fold, about 2 fold to about 40 fold, about 2 fold to about 50 fold, about 2 fold to about 100 fold, about 2 fold to about 200 fold, about 2 fold to about 300 fold, about 2 fold to about 400 fold, about 2 fold to about 500 fold, about 2 fold to about 600 fold, about 2 fold to about 700 fold, about 2 fold to about 800 fold, about 2 fold to about 900 fold, about 2 fold to about 1000 fold, about 4 fold to about 6 fold, about 4 fold to about 8 fold, about 4 fold to about 10 fold, about 4 fold to about 15 fold, about 4 fold to about 20 fold, about 4 fold to about 25 fold, about 4 fold to about 30 fold, about 4 fold to about 35 fold, about 4 fold to about 40 fold, about 4 fold to about 50 fold, about 4 fold to about 100 fold, about 4 fold to about 200 fold, about 4 fold to about 300 fold, about 4 fold to about 400 fold, about 4 fold to about 500 fold, about 4 fold to about 600 fold, about 4 fold to about 700 fold, about 4 fold to about 800 fold, about 4 fold to about 900 fold, about 4 fold to about 1000 fold, about 6 fold to about 8 fold, about 6 fold to about 10 fold, about 6 fold to about 15 fold, about 6 fold to about 20 fold, about 6 fold to about 25 fold, about 6 fold to about 30 fold, about 6 fold to about 35 fold, about 6 fold to about 40 fold, about 6 fold to about 50 fold, about 6 fold to about 100 fold, about 6 fold to about 200 fold, about 6 fold to about 300 fold, about 6 fold to about 400 fold, about 6 fold to about 500 fold, about 6 fold to about 600 fold, about 6 fold to about 700 fold, about 6 fold to about 800 fold, about 6 fold to about 900 fold, about 6 fold to about 1000 fold, about 8 fold to about 10 fold, about 8 fold to about 15 fold, about 8 fold to about 20 fold, about 8 fold to about 25 fold, about 8 fold to about 30 fold, about 8 fold to about 35 fold, about 8 fold to about 40 fold, about 8 fold to about 50 fold, about 8 fold to about 100 fold, about 8 fold to about 200 fold, about 8 fold to about 300 fold, about 8 fold to about 400 fold, about 8 fold to about 500 fold, about 8 fold to about 600 fold, about 8 fold to about 700 fold, about 8 fold to about 800 fold, about 8 fold to about 900 fold, about 8 fold to about 1000 fold, about 10 fold to about 15 fold, about 10 fold to about 20 fold, about 10 fold to about 25 fold, about 10 fold to about 30 fold, about 10 fold to about 35 fold, about 10 fold to about 40 fold, about 10 fold to about 50 fold, about 15 fold to about 20 fold, about 15 fold to about 25 fold, about 15 fold to about 30 fold, about 15 fold to about 35 fold, about 15 fold to about 40 fold, about 15 fold to about 50 fold, about 10 fold to about 100 fold, about 10 fold to about 200 fold, about 10 fold to about 300 fold, about 10 fold to about 400 fold, about 10 fold to about 500 fold, about 10 fold to about 600 fold, about 10 fold to about 700 fold, about 10 fold to about 800 fold, about 10 fold to about 900 fold, about 10 fold to about 1000 fold, about 20 fold to about 25 fold, about 20 fold to about 30 fold, about 20 fold to about 35 fold, about 20 fold to about 40 fold, about 20 fold to about 50 fold, about 20 fold to about 100 fold, about 20 fold to about 200 fold, about 20 fold to about 300 fold, about 20 fold to about 400 fold, about 20 fold to about 500 fold, about 20 fold to about 600 fold, about 20 fold to about 700 fold, about 20 fold to about 800 fold, about 20 fold to about 900 fold, about 20 fold to about 1000 fold, about 25 fold to about 30 fold, about 25 fold to about 35 fold, about 25 fold to about 40 fold, about 25 fold to about 50 fold, about 25 fold to about 100 fold, about 25 fold to about 200 fold, about 25 fold to about 300 fold, about 25 fold to about 400 fold, about 25 fold to about 500 fold, about 25 fold to about 600 fold, about 25 fold to about 700 fold, about 25 fold to about 800 fold, about 25 fold to about 900 fold, about 25 fold to about 1000 fold, about 30 fold to about 35 fold, about 30 fold to about 40 fold, about 30 fold to about 50 fold, about 30 fold to about 100 fold, about 30 fold to about 200 fold, about 30 fold to about 300 fold, about 30 fold to about 400 fold, about 30 fold to about 500 fold, about 30 fold to about 600 fold, about 30 fold to about 700 fold, about 30 fold to about 800 fold, about 30 fold to about 900 fold, about 30 fold to about 1000 fold, about 35 fold to about 40 fold, about 35 fold to about 50 fold, out 35 fold to about 100 fold, about 35 fold to about 200 fold, about 35 fold to about 300 fold, about 35 fold to about 400 fold, about 35 fold to about 500 fold, about 35 fold to about 600 fold, about 35 fold to about 700 fold, about 35 fold to about 800 fold, about 35 fold to about 900 fold, about 35 fold to about 1000 fold, or about 40 fold to about 50 fold, about 40 fold to about 100 fold, about 40 fold to about 200 fold, about 40 fold to about 300 fold, about 40 fold to about 400 fold, about 40 fold to about 500 fold, about 40 fold to about 600 fold, about 40 fold to about 700 fold, about 40 fold to about 800 fold, about 40 fold to about 900 fold, about 40 fold to about 1000 fold greater than a binding affinity of the anti-CD3 antibody or antigen-binding fragment thereof to CD3 at a non-acidic pH. In some embodiments, the binding affinity of the anti-CD3 antibody or antigen-binding fragment thereof to CD3 at acidic pH may be about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, or about 50 fold greater than a binding affinity of the anti-CD3 antibody or antigen-binding fragment thereof to CD3 at a non-acidic pH. In some embodiments, the binding affinity of the anti-CD3 antibody or antigen-binding fragment thereof to CD3 at acidic pH may be at least about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, or about 40 fold greater than a binding affinity of the anti-CD3 antibody or antigen-binding fragment thereof to CD3 at a non-acidic pH. In some embodiments, the binding affinity of the anti-CD3 antibody or antigen-binding fragment thereof to CD3 at acidic pH may be at most about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, or about 50 fold greater than a binding affinity of the anti-CD3 antibody or antigen-binding fragment thereof to CD3 at a non-acidic pH.

In some embodiments, the anti-CD3 antibody or antigen-binding fragment thereof can have an EC₅₀ for binding CD3 of about 50 nM to about 1,500 nM at a non-acidic pH. In some embodiments, the anti-CD3 antibody or antigen-binding fragment thereof can have an EC₅₀ for binding CD3 of about 50 nM to about 100 nM, about 50 nM to about 200 nM, about 50 nM to about 300 nM, about 50 nM to about 400 nM, about 50 nM to about 500 nM, about 50 nM to about 600 nM, about 50 nM to about 700 nM, about 50 nM to about 800 nM, about 50 nM to about 900 nM, about 50 nM to about 1,000 nM, about 50 nM to about 1,500 nM, about 100 nM to about 200 nM, about 100 nM to about 300 nM, about 100 nM to about 400 nM, about 100 nM to about 500 nM, about 100 nM to about 600 nM, about 100 nM to about 700 nM, about 100 nM to about 800 nM, about 100 nM to about 900 nM, about 100 nM to about 1,000 nM, about 100 nM to about 1,500 nM, about 200 nM to about 300 nM, about 200 nM to about 400 nM, about 200 nM to about 500 nM, about 200 nM to about 600 nM, about 200 nM to about 700 nM, about 200 nM to about 800 nM, about 200 nM to about 900 nM, about 200 nM to about 1,000 nM, about 200 nM to about 1,500 nM, about 300 nM to about 400 nM, about 300 nM to about 500 nM, about 300 nM to about 600 nM, about 300 nM to about 700 nM, about 300 nM to about 800 nM, about 300 nM to about 900 nM, about 300 nM to about 1,000 nM, about 300 nM to about 1,500 nM, about 400 nM to about 500 nM, about 400 nM to about 600 nM, about 400 nM to about 700 nM, about 400 nM to about 800 nM, about 400 nM to about 900 nM, about 400 nM to about 1,000 nM, about 400 nM to about 1,500 nM, about 500 nM to about 600 nM, about 500 nM to about 700 nM, about 500 nM to about 800 nM, about 500 nM to about 900 nM, about 500 nM to about 1,000 nM, about 500 nM to about 1,500 nM, about 600 nM to about 700 nM, about 600 nM to about 800 nM, about 600 nM to about 900 nM, about 600 nM to about 1,000 nM, about 600 nM to about 1,500 nM, about 700 nM to about 800 nM, about 700 nM to about 900 nM, about 700 nM to about 1,000 nM, about 700 nM to about 1,500 nM, about 800 nM to about 900 nM, about 800 nM to about 1,000 nM, about 800 nM to about 1,500 nM, about 900 nM to about 1,000 nM, about 900 nM to about 1,500 nM, or about 1,000 nM to about 1,500 nM at a non-acidic pH. In some embodiments, the anti-CD3 antibody or antigen-binding fragment thereof can have an EC₅₀ for binding CD3 at a non-acidic pH of about 50 nM, about 100 nM, about 200 nM, about 300 nM, about 400 nM, about 500 nM, about 600 nM, about 700 nM, about 800 nM, about 900 nM, about 1,000 nM, or about 1,500 nM. In some embodiments, the anti-CD3 antibody or antigen-binding fragment thereof can have an EC₅₀ for binding CD3 at a non-acidic pH of at least about 50 nM, about 100 nM, about 200 nM, about 300 nM, about 400 nM, about 500 nM, about 600 nM, about 700 nM, about 800 nM, about 900 nM, or about 1,000 nM. In some embodiments, the anti-CD3 antibody or antigen-binding fragment thereof can have an EC₅₀ for binding CD3 at a non-acidic pH of at most about 100 nM, about 200 nM, about 300 nM, about 400 nM, about 500 nM, about 600 nM, about 700 nM, about 800 nM, about 900 nM, about 1,000 nM, or about 1,500 nM.

In some embodiments, an acidic pH may comprise pH between 6.0 to 6.9. In some embodiments, the acidic pH may be pH 6.0. In some embodiments, the acidic pH may be pH 6.1. In some embodiments, the acidic pH may be pH 6.2. In some embodiments, the acidic pH may be pH 6.3. In some embodiments, the acidic pH may be pH 6.4. In some embodiments, the acidic pH may be pH 6.5. In some embodiments, the acidic pH may be pH 6.6. In some embodiments, the acidic pH may be pH 6.7. In some embodiments, the acidic pH may be pH 6.8. In some embodiments, the acidic pH may be pH 6.9. In some embodiments, the anti-CD3 antibody or antigen-binding fragment thereof may bind to CD3 at an acidic pH that is less than pH 7.0. In some embodiments, the anti-CD3 antibody or antigen-binding fragment thereof specifically may bind to the CD3 at a pH that is less than pH 6.8. In some embodiments, the anti-CD3 antibody or antigen-binding fragment thereof specifically may bind to the CD3 at a pH that is less than pH 6.5. In some embodiments, the anti-CD3 antibody or antigen-binding fragment thereof specifically may bind to the CD3 at a pH that is less than pH 6.3. In some embodiments, the anti-CD3 antibody or antigen-binding fragment thereof specifically may bind to the CD3 at a pH that is less than pH 6.0. In some embodiments, the anti-CD3 antibody or antigen-binding fragment thereof specifically may bind to the CD3 at a pH that is less than pH 5.8. In some embodiments, the anti-CD3 antibody or antigen-binding fragment thereof specifically may bind to the CD3 at a pH that is less than pH 5.5. In some embodiments, the anti-CD3 antibody or antigen-binding fragment thereof specifically may bind to the CD3 at a pH that is less than pH 5.3. In some embodiments, the anti-CD3 antibody or antigen-binding fragment thereof specifically may bind to the CD3 at a pH that is less than pH 5.0.

In some embodiments, a non-acidic pH may be pH 7.0. In some embodiments, a non-acidic pH may be pH 7.1. In some embodiments, a non-acidic pH may be pH 7.2. In some embodiments, a non-acidic pH may be pH 7.3. In some embodiments, a non-acidic pH may be pH 7.4. In some embodiments, a non-acidic pH may be pH 7.5. In some embodiments, a non-acidic pH may be pH 7.6. In some embodiments, a non-acidic pH may be pH 7.8. In some embodiments, a non-acidic pH may be pH 7.9.

### Antibody Types/Linker

The anti-CD3 antibody or antigen-binding fragment thereof described herein may be a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)₂ fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof.

In some embodiments, the polypeptide described herein may be a multi-chain polypeptide. In some embodiments, the multi-chain polypeptide can be or can include an antibody, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a d-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv₂.

A single chain Fv molecule (scFv) can comprise a VL domain and a VH domain, which may be associated to form a target binding site. These two domains may be further linked by a linker (L). In some embodiments, the anti-CD3 antibody or fragment thereof may comprise a linker peptide. In some instances, the linker may be a flexible linker, a rigid linker, or a cleavable linker. In some embodiments, the linker peptide may link a portion of a heavy chain variable domain to a portion of a light chain variable domain. In some embodiments, a heterodimeric Fc may comprise a first Fc polypeptide linked to the first antigen-binding polypeptide construct (e.g., CD3) with or without a first linker and a second Fc polypeptide linked to the second antigen-binding polypeptide construct (e.g., tumor target) with or without a second linker. The antibody described herein can be optimized for protein expression and yield by changing composition and/or length of the polypeptide linker. In some embodiments, the polypeptide linker can be about 2 amino acids to about 24 amino acids. In some embodiments, the polypeptide linker may about 2 amino acids to about 4 amino acids, about 2 amino acids to about 6 amino acids, about 2 amino acids to about 8 amino acids, about 2 amino acids to about 10 amino acids, about 2 amino acids to about 12 amino acids, about 2 amino acids to about 14 amino acids, about 2 amino acids to about 16 amino acids, about 2 amino acids to about 18 amino acids, about 2 amino acids to about 20 amino acids, about 2 amino acids to about 22 amino acids, about 2 amino acids to about 24 amino acids, about 4 amino acids to about 6 amino acids, about 4 amino acids to about 8 amino acids, about 4 amino acids to about 10 amino acids, about 4 amino acids to about 12 amino acids, about 4 amino acids to about 14 amino acids, about 4 amino acids to about 16 amino acids, about 4 amino acids to about 18 amino acids, about 4 amino acids to about 20 amino acids, about 4 amino acids to about 22 amino acids, about 4 amino acids to about 24 amino acids, about 6 amino acids to about 8 amino acids, about 6 amino acids to about 10 amino acids, about 6 amino acids to about 12 amino acids, about 6 amino acids to about 14 amino acids, about 6 amino acids to about 16 amino acids, about 6 amino acids to about 18 amino acids, about 6 amino acids to about 20 amino acids, about 6 amino acids to about 22 amino acids, about 6 amino acids to about 24 amino acids, about 8 amino acids to about 10 amino acids, about 8 amino acids to about 12 amino acids, about 8 amino acids to about 14 amino acids, about 8 amino acids to about 16 amino acids, about 8 amino acids to about 18 amino acids, about 8 amino acids to about 20 amino acids, about 8 amino acids to about 22 amino acids, about 8 amino acids to about 24 amino acids, about 10 amino acids to about 12 amino acids, about 10 amino acids to about 14 amino acids, about 10 amino acids to about 16 amino acids, about 10 amino acids to about 18 amino acids, about 10 amino acids to about 20 amino acids, about 10 amino acids to about 22 amino acids, about 10 amino acids to about 24 amino acids, about 12 amino acids to about 14 amino acids, about 12 amino acids to about 16 amino acids, about 12 amino acids to about 18 amino acids, about 12 amino acids to about 20 amino acids, about 12 amino acids to about 22 amino acids, about 12 amino acids to about 24 amino acids, about 14 amino acids to about 16 amino acids, about 14 amino acids to about 18 amino acids, about 14 amino acids to about 20 amino acids, about 14 amino acids to about 22 amino acids, about 14 amino acids to about 24 amino acids, about 16 amino acids to about 18 amino acids, about 16 amino acids to about 20 amino acids, about 16 amino acids to about 22 amino acids, about 16 amino acids to about 24 amino acids, about 18 amino acids to about 20 amino acids, about 18 amino acids to about 22 amino acids, about 18 amino acids to about 24 amino acids, about 20 amino acids to about 22 amino acids, about 20 amino acids to about 24 amino acids, or about 22 amino acids to about 24 amino acids. In some embodiments, the polypeptide linker can be about 2 amino acids, about 4 amino acids, about 6 amino acids, about 8 amino acids, about 10 amino acids, about 12 amino acids, about 14 amino acids, about 16 amino acids, about 18 amino acids, about 20 amino acids, about 22 amino acids, or about 24 amino acids. In some embodiments, the polypeptide linker can be at least about 2 amino acids, about 4 amino acids, about 6 amino acids, about 8 amino acids, about 10 amino acids, about 12 amino acids, about 14 amino acids, about 16 amino acids, about 18 amino acids, about 20 amino acids, or about 22 amino acids. In some embodiments, the polypeptide linker may at most about 4 amino acids, about 6 amino acids, about 8 amino acids, about 10 amino acids, about 12 amino acids, about 14 amino acids, about 16 amino acids, about 18 amino acids, about 20 amino acids, about 22 amino acids, or about 24 amino acids. In some embodiments, the linker peptide may comprise (G₄S)ₙ (SEQ ID NO: 91), (SG₄)ₙ (SEQ ID NO: 92), G₄(SG₄)ₙ (SEQ ID NO: 93) or G₂(SG₂)ₙ (SEQ ID NO: 94), wherein n can be 1, 2, 3, 4, 5, 6, 7, 8, 9, or 10. In some embodiments, the linker peptide may comprise VEGGSGGSGGSGGSGGVD (SEQ ID NO: 46).

In some embodiments, the polypeptide may be a single-chain polypeptide. In some embodiments of any of the single-chain polypeptides described herein, the single-chain polypeptide can be or include a BiTe, a (scFv)₂, a nanobody, a nanobody-HSA, a DART, a TandAb, a scDiabody, a scDiabody-CH3, scFv-CH-CL-scFv, a HSAbody, scDiabody-HSA, or a tandem-scFv.

In some embodiments, the antigen-binding domain can be humanized or human. In some embodiments, the antigen-binding domain may not be humanized or not human.

The antibodies of use can be of various isotypes, preferably human IgG1, IgG2, IgG3 or IgG4, more preferably comprising human IgG1 hinge and constant region sequences. The antibodies or fragments thereof can be chimeric human-mouse, humanized (human framework and murine hypervariable (CDR) regions), or fully human, as well as variations thereof, such as half-IgG4 antibodies (referred to as “unibodies. More preferably, the antibodies or fragments thereof may be designed or selected to comprise human constant region sequences that belong to specific allotypes, which may result in reduced immunogenicity when administered to a human subject. Preferred allotypes for administration include a non-G1 m1 allotype (nG1m1), such as Glm3, G1m3,1, G1m3,2 or G1m3,1,2. More preferably, the allotype is selected from the group consisting of the nG1m1, Glm3, nG1m1,2 and Km3 allotypes.

In some embodiments, the T-cell redirecting bispecific antibodies comprising the anti-CD3 and or antigen-binding fragment thereof as described herein may be administered to a subject for diagnosis or treatment of a condition. Exemplary conditions may include, but not limited to, cancer, hyperplasia, neurodegenerative disease, Alzheimer's disease, cardiovascular disease, metabolic disease, vasculitis, viral infection, fungal infection, bacterial infection, diabetic retinopathy, macular degeneration, autoimmune disease, edema, pulmonary hypertension, sepsis, myocardial angiogenesis, plaque neovascularization, restenosis, neointima formation after vascular trauma, telangiectasia, hemophiliac joints, angiofibroma, fibrosis associated with chronic inflammation, lung fibrosis, deep venous thrombosis or wound granulation.

In some embodiments, the anti-CD3 antibody or antigen-binding fragment thereof described herein may be of use for diagnostic or therapy of cancer. Exemplary types of cancers that may be targeted include pancreatic cancer, liver cancer, gastric cancer, lung cancer, colorectal cancer, rectal cancer, thyroid cancer, esophageal cancer, kidney cancer, bladder cancer, prostate cancer, cervical cancer, breast cancer, skin cancer, epithelial cancer, brain cancer, or ovarian cancer, acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer.

In some embodiments, the cancer may comprise a solid tumor, such as adenocarcinoma, colon adenocarcinoma, rectal adenocarcinoma, pancreatic adenocarcinoma, glioblastoma, neuroendocrine tumors, colorectal carcinoma, gastric carcinoma, esophageal carcinoma, neuroblastoma, or osteosarcoma.

In some embodiments, the bsAbs comprising the anti-CD3 antibody or antigen-binding fragment thereof as described herein may comprise additional tumor-associated antigens that may be targeted. The tumor-associated antigen can be, but are not limited to, alpha-fetoprotein (AFP), α-actinin-4, A3, antigen specific for A33 antibody, ART-4, B7, Ba 733, BAGE, BrE3-antigen, CA125, CAMEL, CAP-1, carbonic anhydrase IX, CASP-8/m, CCL19, CCL21, CD1, CD1aa, CD2, CD3, CD4, CD5, CD8, CD11A, CD14, CD15, CD16, CD17, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, CDH1, CDH6, CDH17, CDH19, CDH179b, HIF-1α, colon-specific antigen-p (CSAp), CEA (CEACAM5), CEACAM6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, S100, survivin, survivin-2B, TAC, TAG-72, tenascin, TRAIL receptors, TNF-α, Tn antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker and an oncogene product.

Exemplary antibodies that may be used in combination with an anti-CD3 antibody or antigen-binding fragment thereof include, but are not limited to, hA19 (anti-CD19), hR1 (anti-IGF-1R), hPAM4 (anti-mucin), hA20 (anti-CD20), hIMMU31 (anti-AFP), hLL1 (anti-CD74), hLL2 (anti-CD22), hMu-9 (anti-CSAp), hL243 (anti-HLA-DR), hMN-14 (anti-CEACAM5), hMN-15 (anti-CEACAM6), hRS7 (anti-EGP-1), hMN-3 (anti-CEACAM6), Ab124 and Ab125 (anti-CXCR4), the Examples section of each cited patent or application incorporated herein by reference. Alternative antibodies that may be attached to the anti-CD3 for treatment of various disease states include, but are not limited to, abciximab (anti-glycoprotein IIb/IIIa), alemtuzumab (anti-CD52), bevacizumab (anti-VEGF), cetuximab (anti-EGFR), gemtuzumab (anti-CD33), ibritumomab (anti-CD20), panitumumab (anti-EGFR), rituximab (anti-CD20), tositumomab (anti-CD20), trastuzumab (anti-ErbB2), lambrolizumab (anti-PD-1 receptor), nivolumab (anti-PD-1 receptor), ipilimumab (anti-CTLA-4), abagovomab (anti-CA-125), adecatumumab (anti-EpCAM), atlizumab (anti-IL-6 receptor), benralizumab (anti-CD125), obinutuzumab (GA101, anti-CD20), CC49 (anti-TAG-72), AB-PG1-XG1-026 (anti-PSMA), D2/B (anti-PSMA), tocilizumab (anti-IL-6 receptor), basiliximab (anti-CD25), daclizumab (anti-CD25), efalizumab (anti-CD11a), GA101 (anti-CD20; Glycart Roche), atalizumab (anti-α4 integrin), omalizumab (anti-IgE); anti-TNF-α antibodies such as CDP571, MTNFAI, M2TNFAI, M3TNFAI, M3TNFABI, M302B, M303 (Thermo Scientific, Rockford, Ill.), infliximab (Centocor, Malvern, Pa.), certolizumab pegol (UCB, Brussels, Belgium), anti-CD40L (UCB, Brussels, Belgium), adalimumab (Abbott, Abbott Park, Ill.), BENLYSTA® (Human Genome Sciences); antibodies for therapy of Alzheimer's disease such as Alz 50, gantenerumab, solanezumab and infliximab; anti-fibrin antibodies like 59D8, T2G1s, MH1; anti-CD38 antibodies such as MOR03087 (MorphoSys AG), MOR202 (Celgene), HuMax-CD38 (Genmab) or daratumumab (Johnson & Johnson); anti-HIV antibodies such as P4/D10, Ab 75, Ab 76, Ab 77, as well as the anti-HIV antibodies.

### Method of Making Polypeptides

Also provided herein are methods of producing an polypeptide that include: (a) culturing a cell (e.g., any of the cells described herein) including any of the nucleic acids encoding any of the polypeptides described herein, or any of the expression vectors described herein that include nucleic acid encoding any of the polypeptides described herein, in a culture medium under conditions sufficient to allow for the production of the polypeptide; and (b) harvesting the polypeptide from the host cell or the culture medium. In some embodiments of any of the methods described herein, the method further includes isolating the polypeptide (e.g., through performance of one or more column chromatography steps, ultrafiltration/diafiltration, and/or viral inactivation). In some embodiments of any of the methods described herein, the method further includes formulating the isolated polypeptide into a composition (e.g., a pharmaceutical composition).

Any of the polypeptides described herein can be produced by any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell. For example, a host cell can be a CHO cell or a HEK cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or expression vectors (e.g., any of the exemplary expression vectors described herein (e.g., an AAV vector)) into cells (e.g., mammalian cells) are known in the art. Non-limiting examples of methods that can be used to introduce a nucleic acid (e.g., DNA, RNA, ssRNA, siRNA, microRNA, or mRNA) and/or an expression vector (e.g., any of the exemplary expression vectors described herein (e.g., an AAV vector) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

Some methods described herein further include isolating or purifying the polypeptide from cell culture medium or from a cell (e.g., a mammalian cell) using techniques well-known in the art (e.g., ion exchange chromatography (anionic or cation), metal-affinity chromatography, ligand-affinity chromatography, size exclusion chromatography, hydrophobic interaction chromatography, and precipitation (e.g., ammonium sulfate precipitation, polyethylene glycol precipitation).

### Method of Treatment

Described herein is the pharmaceutical composition for use in a therapy includes an antibody monotherapy, antibody-drug conjugate (ADC) therapy, T cell-engaging immunotherapy, or CAR-T therapy. In some embodiments, the pharmaceutical compositions may further comprise at least one additional therapeutic agent.

In some embodiments, compositions comprising antibodies can be provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, compositions comprising antibodies may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1. In some embodiments, the composition described herein may be manufactured as a medicament for treatment of cancer.

### Pharmaceutical Composition

Also provided herein are compositions (e.g., pharmaceutical compositions) that include any of the polypeptides, vectors, or nucleic acids described herein. Any of the pharmaceutical compositions can include any of the polypeptides, vectors, or nucleic acids described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the vectors or nucleic acids described herein into a cell (e.g., a mammalian cell) (e.g., a liposome or cationic lipid).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

The pharmaceutical compositions provided herein can be, e.g., formulated to be compatible with their intended route of administration. In some embodiments, the compositions are formulated for subcutaneous, intramuscular, or intravenous administration. In some examples, the compositions include a therapeutically effective amount of any of the polypeptides, vectors, or nucleic acids described herein. Single or multiple administrations of any of the pharmaceutical compositions described herein can be given (e.g., administered) to a subject depending on, for example, the frequency and the dosage required and tolerated by the subject. A dosage of the pharmaceutical composition including any of the polypeptides described herein, any of the vectors described herein, or any of the nucleic acids described herein should provide a sufficient quantity to effectively ameliorate or treat symptoms, conditions or diseases.

### Kits

A kit-of-parts comprising a pharmaceutical composition together with instructions for use is further provided. For convenience, the kit-of-parts may comprise reagents in predetermined amounts with instructions for use.

In some embodiments, disclosed herein are kids comprising an anti-CD3 antibody or antigen-binding fragment thereof disclosed herein. In some embodiments, a kit can be a diagnostic kit. In some embodiments, a kit comprises an anti-CD3 antibody or antigen-binding fragment thereof disclosed herein and instructions for use. In some embodiments, a kit comprises means for measuring an anti-CD3 antibody or antigen-binding fragment thereof level in a sample and instructions for use. A kit may provide a unit or device for obtaining a sample from a subject (e.g., a device with a needle coupled to an aspirator).

A kit may include a plurality of syringes, ampules, foil packets, or blister packs, each containing a single unit dose of a kit component described herein. Containers of a kit may be airtight, waterproof (e.g., impermeable to changes in moistures or evaporation), and/or light-tight. A kit may include a device suitable for administration of the components, e.g., a syringe, inhalant, pipette, forceps, measured spoon, dropper (e.g., eye dropper), swab (e.g., a cotton swab or wooden swab), or any such delivery device. In some embodiments, the device may be a medical implant device, e.g., packaged for surgical insertion. A kit disclosed herein may comprise one or more reagents or instruments which enable the method to be carried out. In some embodiments, reagents or instruments include one or more of the following: suitable buffer(s) (aqueous solutions), a support comprising wells on which quantitative reactions can be done. A kit may be a specific kit for a specific tissue sample. Further, a kit disclosed herein may comprise a control. In some embodiments, the kid may comprise any of the compositions (e.g., pharmaceutical compositions) described herein which include any of the nucleic acids, any of the polypeptides, or any of the vectors described herein. In some embodiments, a kit can include a solid composition (e.g., a lyophilized composition including any of the vectors, polypeptides, or nucleic acids described herein) and a liquid for solubilizing the lyophilized composition. In some embodiments, the kit includes a vial including any of the pharmaceutical compositions described herein (e.g., formulated as an aqueous pharmaceutical composition).

In addition to the above components, instructions for use may be provided in a kit. These instructions may be presented in the kit in a variety of forms, such as printed information on a suitable medium or substrate (e.g., a piece or pieces of paper on which the information is printed), in the packaging of the kit, in a package insert, etc. In some embodiments, instructions for use can be provided on a computer readable medium (e.g., jump/thumb drive, CD, etc.), or which the information has been recorded, or at a website address which may be used via the internet to access the information at a website.

### Device

Another aspect of the disclosure provides a pre-filled syringe or autoinjector device, comprising an anti-CD3 antibody or antigen-binding fragment thereof or a composition described herein. In some embodiments, a composition stored in a container, pre-filled syringe, injector or autoinjector device contains an anti-CD3 antibody or antigen-binding fragment thereof disclosed herein.

### Arrays

Disclosed herein are supports comprising an anti-CD3 antibody or antigen-binding fragment thereof disclosed herein. A support can be a solid support. A support may take a variety of configurations ranging from simple to complex, depending on the intended use of the support. A support can have an overall slide or plate configuration, such as a rectangular or disc configuration. A standard microplate configuration can be used. In some embodiments, the surface may be smooth or substantially planar, or have irregularities, such as depressions or elevations. In some embodiments, a support may have a rectangular cross-sectional shape, having a length of from about 10-200 mm, 40-150 mm, or 75-125 mm; a width of from about 10-200 mm, 20-120 mm, or 25-80 mm, and a thickness of from about 0.01-5.0 mm, 0.1-2 mm, or 0.2 to 1 mm.

In some embodiments, a support can be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A support as described herein can be formed of any suitable material, including metals, metal oxides, semiconductors, polymers (particularly organic polymers in any suitable form including woven, nonwoven, molded, extruded, cast, etc.), silicon, silicon oxide, and composites thereof. A support can be an array. In some embodiments, a support comprises an array. An array can comprise an ordered spatial arrangement of two or more discrete regions. An array can comprise anti-CD3 antibodies or antigen-binding fragments thereof located at known or unknown discrete regions. Row and column arrangements of arrays can be selected due to the relative simplicity in making such arrangements. The spatial arrangement can, however, be essentially any form selected by the user, and optionally, in a pattern. Areas of an array may be any convenient shape, including circular, ellipsoid, oval, annular, or some other analogously curved shape, where the shape may, in certain embodiments, be a result of the particular method employed to produce the array.

In some embodiments, a support can be planar. In some instances, a support can be spherical. In some instances, a support can be a bead. In some instances, a support can be magnetic. In some embodiments, a magnetic support can comprises magnetite, maghemitite, FePt, SrFe, iron, cobalt, nickel, chromium dioxide, ferrites, or mixtures thereof. In some embodiments, a support can be nonmagnetic. In some embodiments, the nonmagnetic support can comprise a polymer, metal, glass, alloy, mineral, or mixture thereof. In some instances, a nonmagnetic material can be a coating around a magnetic support. In some instances, a magnetic material may be distributed in the continuous phase of a magnetic material. In some embodiments, the support comprises magnetic and nonmagnetic materials. In some instances, a support can comprise a combination of a magnetic material and a nonmagnetic material. In some embodiments, an anti-CD3 antibody or antigen-binding fragment thereof disclosed herein is directly or indirectly associated with a support disclosed herein.

### Computer Control System

The present disclosure provides computer control systems that are programmed to implement methods of the disclosure. In some embodiments, a computer system is programmed or otherwise configured to interface with an apparatus that is configured to detect CD3 and/or binding of an anti-CD3 antibody or antigen-binding fragment thereof disclosed herein to moiety. The computer system can be an electronic device of a user or a computer system that is remotely located with respect to the electronic device. The electronic device can be a mobile electronic device.

In some embodiments, a computer system includes a central processing unit (CPU, also “processor” and “computer processor” herein, which can be a single core or multi core processor, or a plurality of processors for parallel processing.

In some embodiments, a computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. In some embodiments, the memory, storage unit, interface and peripheral devices are in communication with the CPU through a communication bus, such as a motherboard. In some embodiments, the storage unit can be a data storage unit (or data repository) for storing data. In some embodiments, the computer system is operatively coupled to a computer network (“network”) with the aid of the communication interface. In some embodiments, the network can be the Internet, an internet and/or extranet, or an intranet and/or extranet that is in communication with the Internet. In some embodiments, the network is a telecommunication and/or data network. The network can include one or more computer servers, which can enable distributed computing, such as cloud computing. In some embodiments, the network, in some cases with the aid of the computer system can implement a peer-to-peer network, which may enable devices coupled to the computer system to behave as a client or a server. In some embodiments, the CPU executes a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback. In some embodiments, the CPU can be part of a circuit, such as an integrated circuit. One or more other components of the system can be included in the circuit. In some cases, the circuit is an application specific integrated circuit (ASIC). In some embodiments, the storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet. In some embodiments, the computer system communicates with one or more remote computer systems through the network. For instance, the computer system can communicate with a remote computer system of a user. Examples of remote computer systems include personal computers (e.g., portable PC), slate or tablet PC's telephones, Smart phones, or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory. In some embodiments, the code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

A machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution. In some embodiments, a computer system disclosed herein can include or be in communication with an electronic display that comprises a user interface (UI) for providing, for example, one or more results (immediate results or archived results from a previous experiment), one or more user inputs, reference values from a library or database, or a combination thereof. Examples of UIs include, without limitation, a graphical user interface (GUI) and web-based user interface.

Further, methods and systems of the present disclosure can be implemented by way of one or more algorithms. An algorithm can be implemented by way of software upon execution by the central processing unit. The algorithm can, for example, determine optimized conditions via supervised learning to optimize conditions such as a buffer type, a buffer concentration, a temperature, an incubation period, thresholds, diagnostic/prognostic indications for methods disclosed herein.

### EMBODIMENTS

Embodiment 1. An anti-CD3 antibody or antigen-binding fragment thereof, comprising a polypeptide having at least 90% sequence identity to SEQ ID NO: 1, wherein the polypeptide comprises at least one CDR having an amino acid substitution with histidine, aspartic acid, or glutamic acid,
optionally wherein:
1. the polypeptide comprises a sequence at least 90% identical to SEQ ID NOs: 2-21; and/or
2. the polypeptide comprises a sequence at least 90% identical to SEQ ID NOs: 605-614; and/or
3. the anti-CD3 antibody or antigen-binding fragment thereof comprises HCDR1 comprising a sequence selected from a group consisting of SEQ ID NOs: 47-48; HCDR2 comprising a sequence selected from a group consisting of SEQ ID NOs: 49-59; and HCDR3 comprising a sequence selected from a group consisting of SEQ ID NOs: 60-61; and/or
4. the anti-CD3 antibody or antigen-binding fragment thereof comprises LCDR1 comprising a sequence selected from a group consisting of SEQ ID NOs: 62-63; LCDR2 comprising a sequence of SEQ ID NO: 64; and LCDR3 comprising a sequence selected from a group consisting of SEQ ID NOs: 65-69 or 618.

Embodiment 2. The anti-CD3 antibody or antigen-binding fragment thereof according to any one of the Embodiments provided herein, wherein the binding affinity for CD3 at an acidic pH is 30-fold or more greater than the binding affinity of the same anti-CD3 antibody to CD3 at a non-acidic pH,
optionally wherein:
1. the binding affinity for CD3 at an acidic pH is 50-fold or more greater than the binding affinity of the same anti-CD3 antibody to CD3 at a non-acidic pH; and/or
2, wherein the acidic pH is 6.0, and/or
3. the non-acidic pH is 7.4.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application.

### Example 1: Cell-Free Expression and Validation of Anti-CD3 scFv Binding and Activity

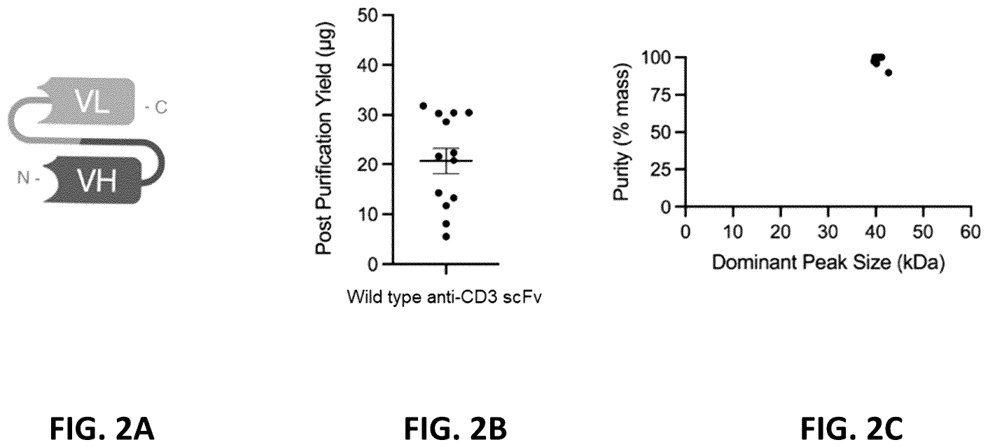

The wild type anti-CD3 scFV sequence, OKT scFv, was sourced from the sequence of the approved CD3×CD19 bsAb TCE BLINCYTO. The coding sequence in VH-VL configuration with 5×(GGS) interdomain linker as shown in FIG. 2A, was codon optimized for bacterial expression and was cloned into a cell free expression vector. The wild type molecule was expressed, purified with single-step affinity purification, quantitated by spectrophotometry, and characterized for biophysical properties. As shown in FIG. 2B, standard post-purification yields were 20.7±0.7 μg with an average A₂₆₀/A₂₈₀ ratio of 0.6±0.08 μg. Average of thermostability measurements indicates a Tm of approximately 71° C. As shown in FIG. 2C, in CE-SDS assay, the wild type anti-CD3 scFv migrated about 10 kDa slower than the calculated MW and more than 98% of the sample mass migrated at this single apparent fragment size on average. To understand the binding properties of the CFPS-expressed wild type molecule, FACS using CD3+/+ Jurkat E6.1 cells and CD3−/− Jurkat cells to confirm specific, on-target binding were performed. Binding of antibodies to CD3+ cells was determined by flow cytometry (FACS) with standard methods. Jurkat E6.1 cells (ATCC TIB-152) were maintained in culture under controlled conditions. Serial dilutions of antibodies were prepared by diluting concentrated antibody stocks to twice the final desired assay concentration in PBS-based FACS buffer adjusted to pH 7.4 or pH 6.0 (2% (v/v) FBS, 4 mM EDTA in 1×PBS). Cells were harvested from culture by centrifugation at 4000 rcf for 4 min. Culture media was aspirated, and cells were washed with sterile PBS twice. Cells were then resuspended in PBS containing eFluor 450 (1:1000) for 40 min at 4C in the dark. 2×105 cells were then aliquoted into each well of a 96 well plate. Equal volumes of serially diluted antibody was added to each well. After a 1 h incubation at 4C, cells were washed twice in pH-specific FACS buffer. Strep-Tactin®XT conjugate DY-649 (IBA 2-1568-050) was diluted to 1:5000 in pH-specific FACS buffer, and washed cells were then resuspended in 80 μl of this secondary-containing buffer. After a 1 h incubation at 4C, cells were again washed. Stained cells were then fixed prior to cytometry by resuspension in 100 μl of pH-specific FACS buffer containing 0.5% (v/v) paraformaldehyde. Samples were then analyzed on a NovoCyte Penteon Cytometer, and data were analyzed with standard techniques using FlowJo and PRISM. The selectivity index of a given antibody represents the ratio of EC₅₀ at pH 7.4 vs pH 6.0.

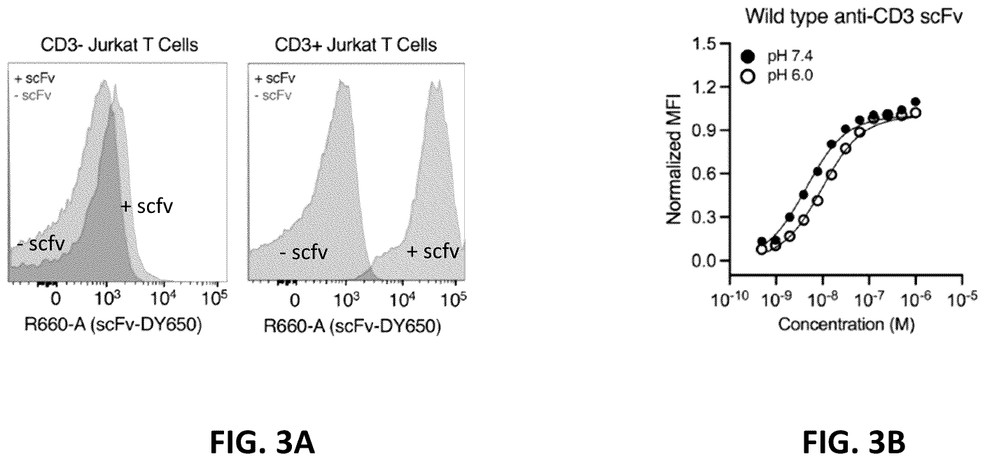

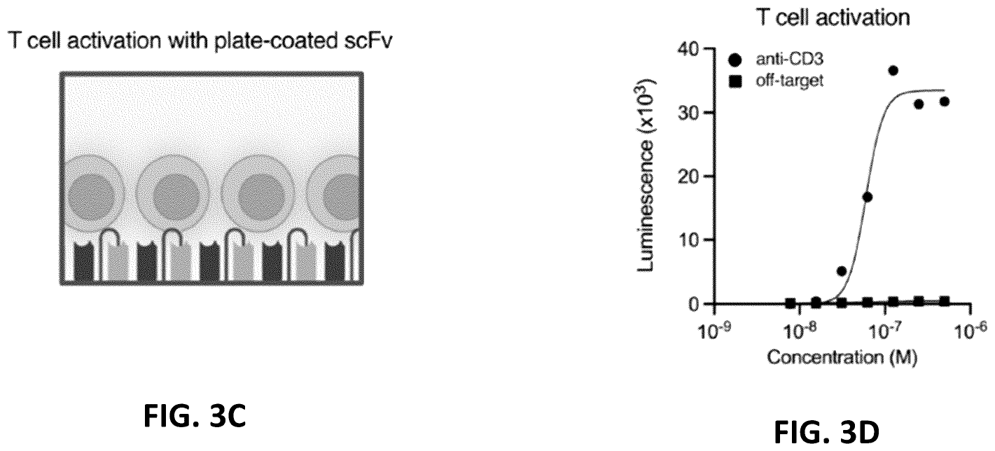

On-target binding experiments demonstrated that the wild type scFv binding is specific to hCD3 (FIG. 3A) and replicate binding experiments estimate an EC₅₀ of 14.9±7.4 nM. Subsequent analysis using cells stained in PBS pH 6.0 show very similar binding with an EC₅₀ of 12.5±4.6 nM (FIG. 3B).

To confirm the ability of the wild type anti-CD3 scFv to activate T cells via CD3 binding, Jurkat T cells modified to express a luciferase reporter gene under the control of the nuclear factor of activated T cells (NFAT) promoter were incubated overnight in wells of assay plates that had been passively coated with serial dilutions of anti-CD3 scFv or an off-target control scFv (FIG. 3C).

As shown in FIG. 3D, on-target anti-CD3 scFv robustly activated NFAT transcriptional activity in a dose-dependent manner whereas off-target scFv did not.

This example demonstrates that wild type anti-CD3 scFv specifically binds to hCD3 and shows similar binding activity at pH 6.0 and pH 7.4.

### Example 2. Cell-Free Expression of Anti-CD3 Bispecific Antibodies and Validation of Activity

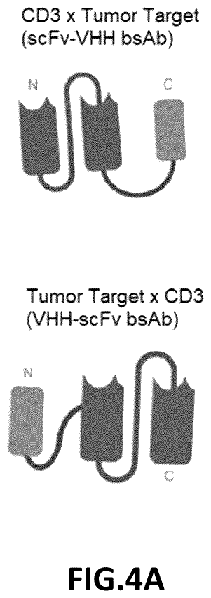

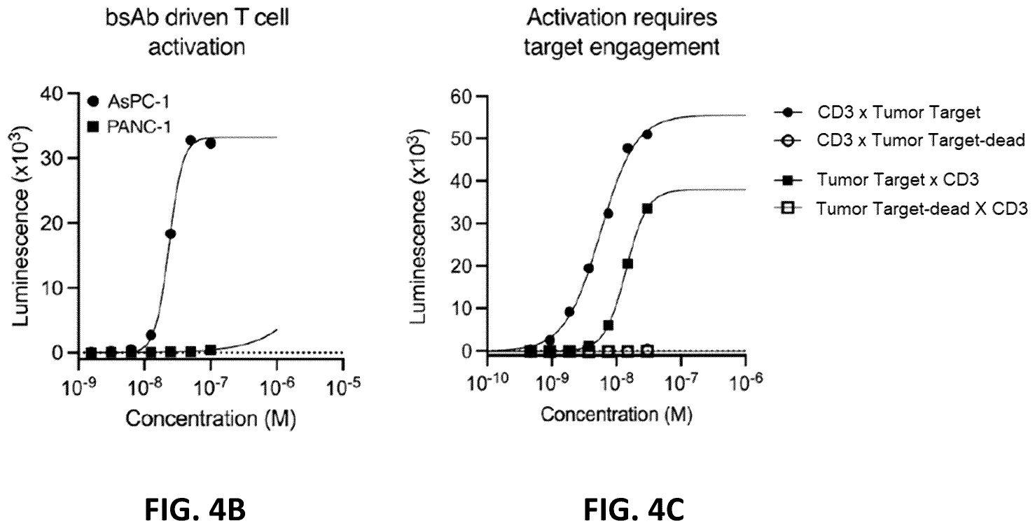

To validate the ability of the wild type anti-CD3 scFv (OKT3) to activate T cells in response to a target antigen in the minimal BiTE format, the wild type anti-CD3 scFv with a proprietary VHH targeting a tumor associated antigen overexpressed and uniquely accessible in an array of alimentary tract cancers, was engineered. Specifically scFv-VHH and VHH-scFv configurations were expressed with 1×(GGGGS) peptide linker (SEQ ID NO: 95) (FIG. 4A). These molecules were expressed overnight in the cell free expression systems and purified with single-step affinity chromatography. Average post-purification yield was lower than the scFv alone (8.2 μg vs 20.7 μg). Purity by CE-SDS demonstrated that more than 95% of sample mass occurred at the dominant peak. The addition of the single VHH domain antibody shifted the apparent molecule size by the expected molecular weight of the VHH antibody (˜15 kDa).

The ability of the wild type anti-CD3 scFv to activate T cells in the context of a minimal drug-like BiTE bsAb was assessed by co-culturing (E:T 1:1) serial dilutions of antibodies with Jurkat(NFAT-Luc) cells and AsPC-1 and PANC-1 cell lines. Serial dilutions of antibodies were prepared PBS adjusted to pH 6.2 or pH 7.4 and adsorbed overnight in 96 well microplates that were passively blocked with 1% BSA in PBST (0.1% (v/v) Tween-20 in 1×PBS, pH 7.4). Coated wells were then washed with PBST to remove excess unbound antibody. Cells were harvested by centrifugation, and 4×10⁴ cells were added to each well in media adjusted to pH 6.2 or pH 7.4. Cells were incubated for 24 h at 37C, and luminescent signal corresponding to T cell activation was measured using ONE-Glo reagent (BPS Biosciences) according to manufacturer recommendations. Luminescence was detected using a Spectramax plate reader (Molecular Devices).

AsPC-1 but not PANC-1 target cells led to robust T cell luciferase expression, consistent with target- and antibody-dependent T cell activation (FIG. 4B).

To further demonstrate BiTE specificity, a bsAb expressing a dead version of the anti-tumor target VHH with a single mutation known to ablate binding was engineered. As shown in FIG. 4C, only the bsAb containing the functional VHH activated T cells, demonstrating T cell activation is dependent on antibody-driven ligation of tumor and T cell targets, and soluble bsAb on its own is incapable of activating T cells.

This example demonstrates that the wild type anti-CD3 scFv can bind and activate T cells in drug-like bsAb TCE formats.

### Example 3. Engineering Anti-CD3 scFv Variants with Acid-Specific Binding

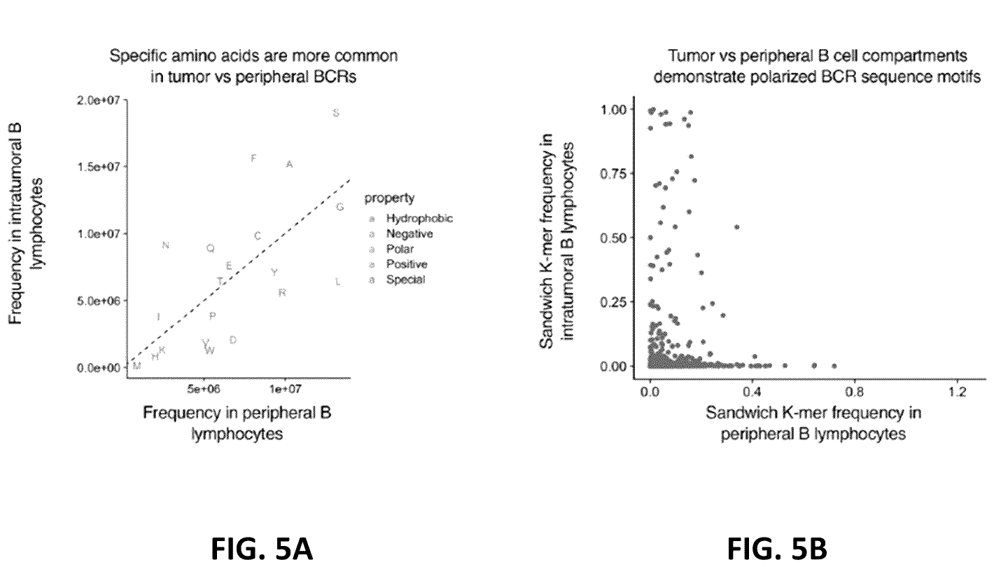

The wild type anti-CD3 scFv sequence was optimized to reduce on-target binding at pH 7.4 while preserving binding at pH 6.0. This optimization was designed to mitigate spurious T cell activation in the periphery, which drives serious off-tumor toxicities, and concentrate the bsAb TCE in the tumor by avoiding T cell binding in the periphery. To engineer an antibody capable to bind to the target in pH-dependent manner, models of antibody CDR sequence space were built by incorporating three design principles: (1) Single and combinatorial histidine scanning; (2) Single and combinatorial aspartate/glutamate scanning; (3) Sequence patterns occurring in tumor infiltrating B lymphocytes and not in peripheral circulating B cells (FIG. 5A) Frequencies of all sandwich 3-mers (e.g. XAX, XKX where A or K is the amino acid of interest and Xs are flanking amino acids, each dot is a unique 3-mer) between intratumoral and peripheral B cell repertoires show stark differences in sequence utilization. (FIG. 5B). Separately, an antibody library was designed for phage display and enriched for variants that prefer pH 6.0 over pH 7.4.

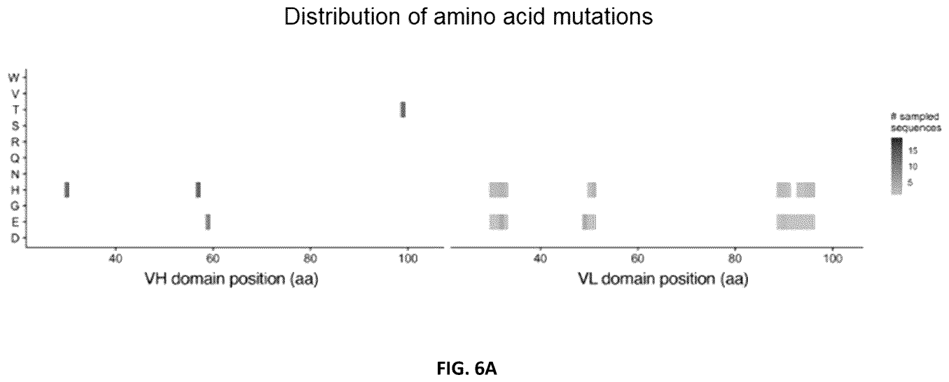

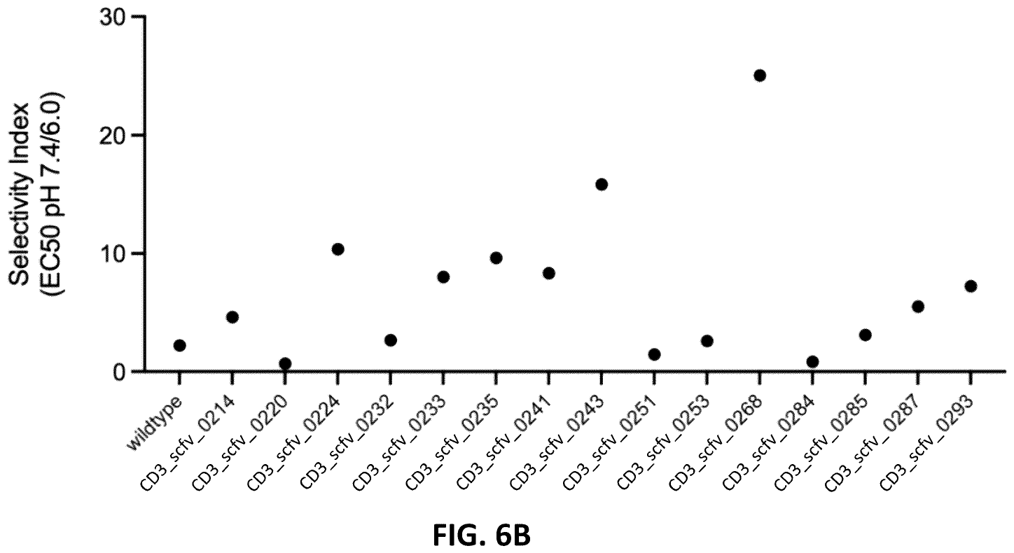

In Round 3 of the optimization, binding could be detected at pH 6.0 for 20 constructs out of the 44 design constructs. The average SI for binding constructs was 4.47 (FIG. 6B).

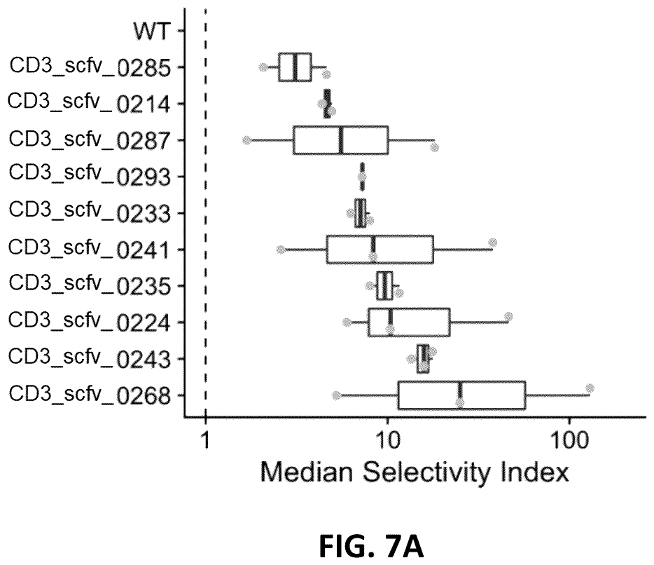

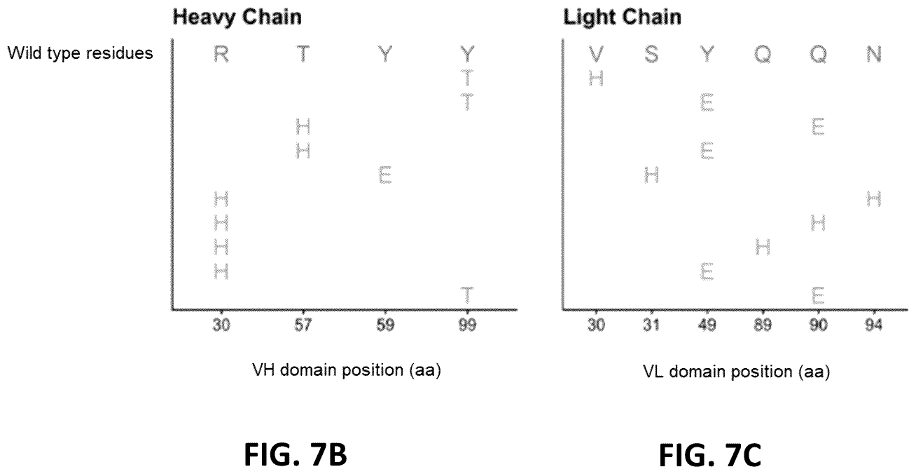

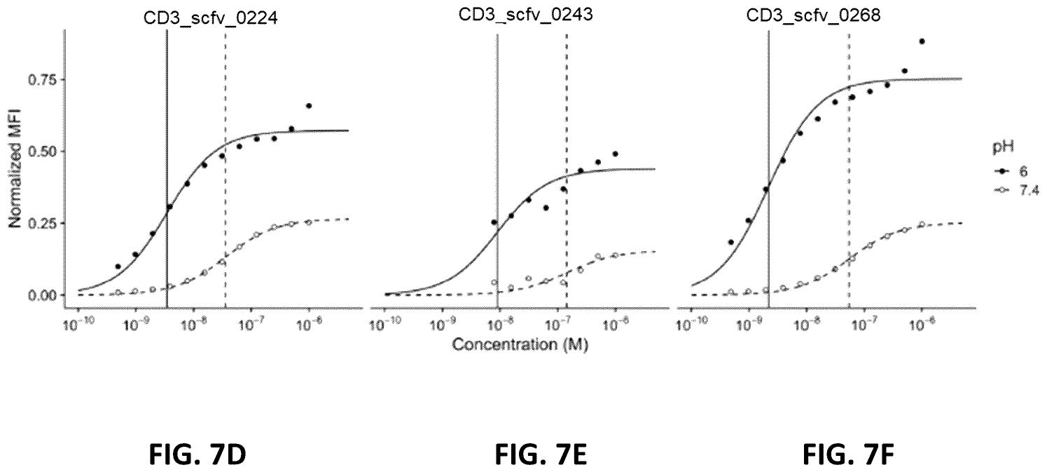

Anti-CD3 scFv variants 0268, 0243, and 0224 showed a higher selectivity index represented by the ratio of EC₅₀ estimates determined for binding at pH 7.4 over pH 6.0 (FIGS. 7D-7F). Anti-CD3 scFv variant 0268 containing Y99T in the VH domain and Q89E in the VL domain had an SI of 25.04. Anti-CD3 scFv variant 0243 containing R30H in the VH domain and Y48E in the VL domain had an SI of 15.82. Anti-CD3 scFv variant 0224 contained the mutation R30H and Q89H in the VH domain and had an SI of about 10.37.

### Example 4. Validation of Selective Anti-CD3 Variants

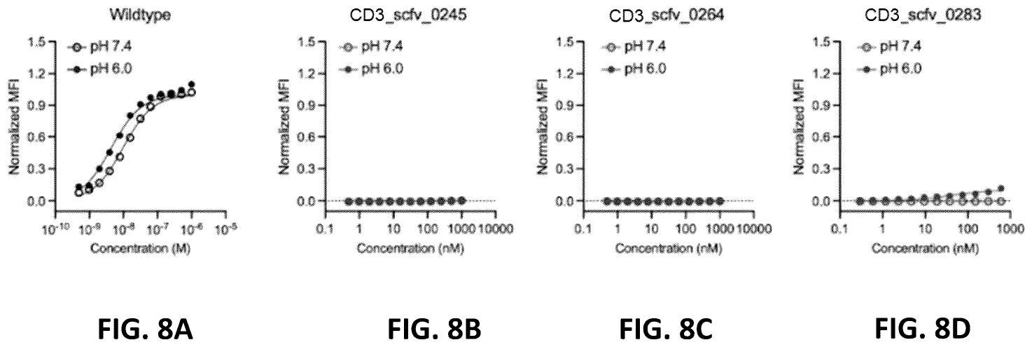

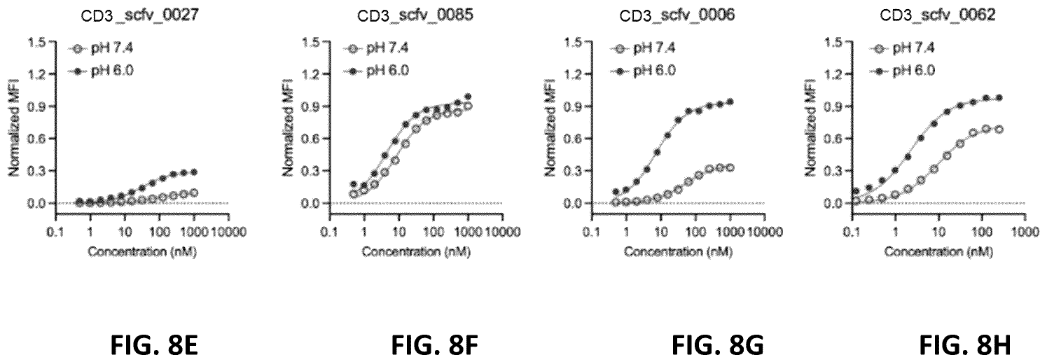

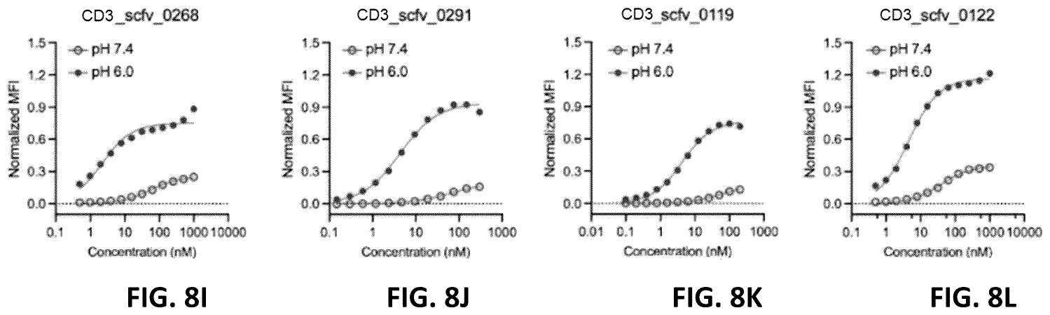

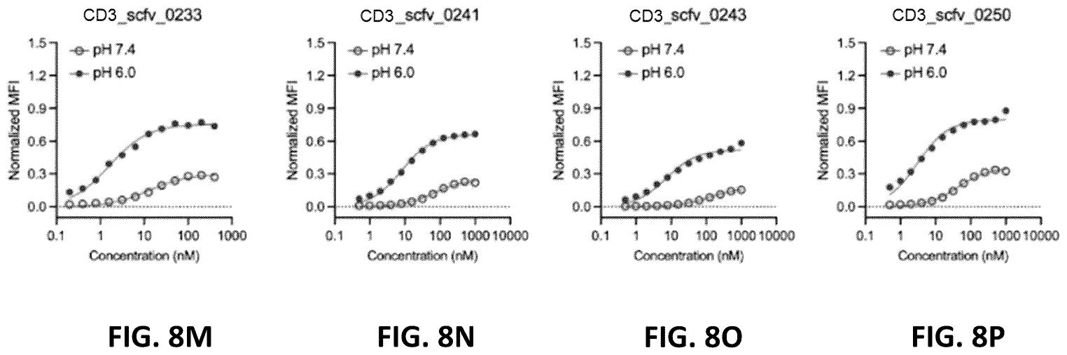

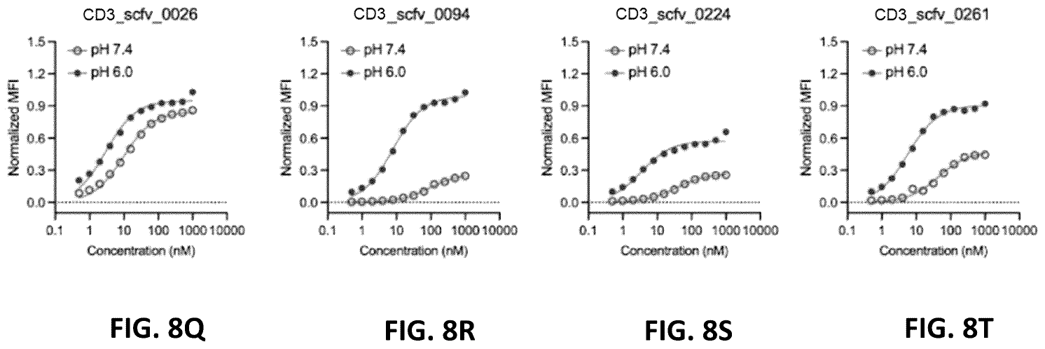

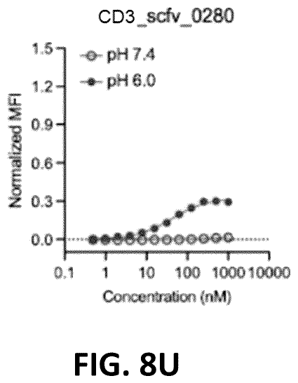

To validate forementioned 20 variants, two negative controls and many of the most apparently selective clones, utilized a 12 point serial dilution in biological duplicate. These results corroborate the previously obtained binding profiles and provide clear evidence that the constructs demonstrated a range of pH dependent binding behavior (FIGS. 8A-8U). In these validation replicates, the binding traces of all variants seem to be uniformly left shifted to higher apparent affinity at both pHs. Selectivity indices for each variant were within 5-35 fold (Table 3). Anti-CD3 scFv variant 0280 remained the most selective with a SI>30 and no detectable binding at pH 7.4.

The measured selectivity index as a ratio of maximum MFI between pH 6.0 and pH 7.4 were correlated (R=0.78).

TABLE 3
Summary of EC₅₀ and maximum MFI
of anti-CD3 constructs (Round 3)

| Construct Name | EC₅₀ pH 6.0 | EC₅₀ pH 7.4 | SI |
|---|---|---|---|
| CD3_scfv_0280 | 3.02E−08 | 1.00E−06 | 33.12 |
| CD3_scfv_0268 | 4.05E−09 | 4.53E−08 | 25.04 |
| CD3_scfv_0243 | 7.21E−09 | 1.35E−07 | 15.82 |
| CD3_scfv_0291 | 5.17E−09 | 7.82E−08 | 14.27 |
| CD3_scfv_0250 | 7.21E−09 | 4.89E−08 | 14.15 |
| CD3_scfv_0119 | 1.08E−08 | 1.00E−06 | 12.89 |
| CD3_scfv_0224 | 3.47E−09 | 2.36E−08 | 10.37 |
| CD3_scfv_0247 | 2.03E−09 | 1.44E−07 | 10.17 |
| CD3_scfv_0235 | 1.90E−09 | 1.83E−08 | 9.64 |
| CD3_scfv_0104 | 2.75E−08 | 8.05E−07 | 8.86 |
| CD3_scfv_0241 | 1.22E−08 | 5.98E−08 | 8.33 |
| CD3_scfv_0233 | 3.66E−09 | 5.37E−08 | 8.02 |
| CD3_scfv_0261 | 5.75E−09 | 3.14E−08 | 7.61 |
| CD3_scfv_0293 | 2.64E−08 | 4.38E−07 | 7.26 |
| CD3_scfv_0060 | 4.46E−08 | 3.23E−07 | 7.25 |
| CD3_scfv_0094 | 1.35E−08 | 1.03E−07 | 6.82 |
| CD3_scfv_0122 | 4.93E−09 | 5.00E−08 | 6.68 |
| CD3_scfv_0006 | 7.31E−09 | 7.63E−08 | 6.27 |
| CD3_scfv_0287 | 2.97E−08 | 1.65E−07 | 5.54 |
| CD3_scfv_0099 | 2.28E−08 | 1.16E−07 | 5.1 |

### Example 5. Effects of Acid-Specific Binding on T Cell Activation and Cytotoxicity

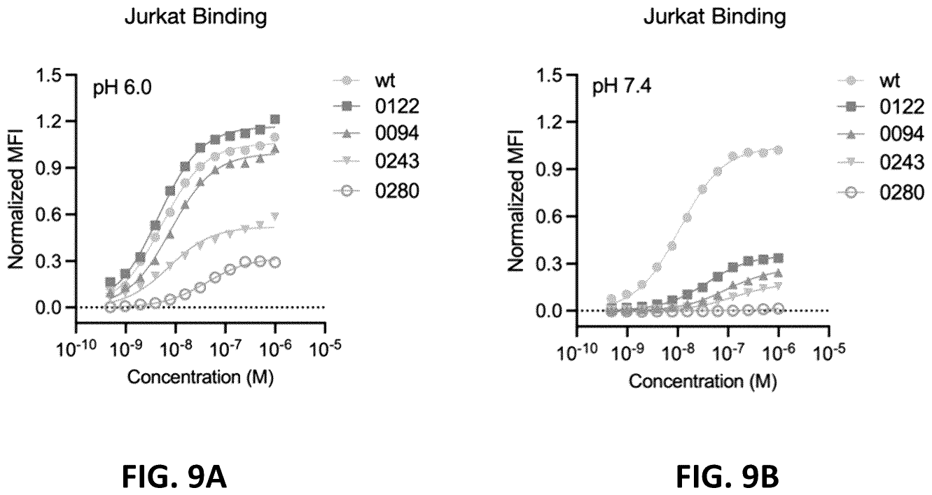

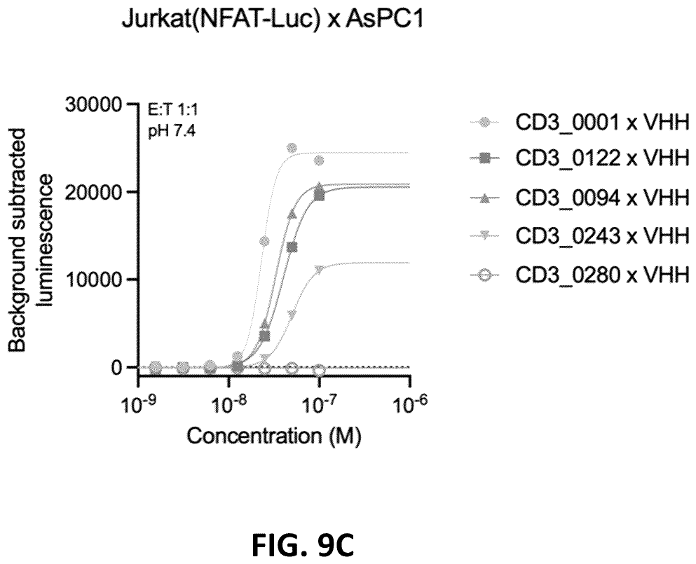

Based on this repertoire of anti-CD3 constructs demonstrating various degrees of acid-specific binding, bispecific antibodies using the minimal BiTE® format was generated with the anti-tumor target antibody. Specifically, the TCE was built using variants that displayed a range of cell binding at pH 7.4 and retained binding at pH 6.0 (FIGS. 9A and 9B), then expressed and characterized these CD3×Tumor Target bsAb TCEs using T cell activation assay involving Jurkat(NFAT-Luc) and AsPC-1 co-culture (FIG. 9C). Interestingly, despite a relatively small difference in binding at pH 7.4 between the variant clones and all selected variants displaying drastically reduced binding at pH 7.4 relative to the wild type sequence (FIG. 9B), bsAbs containing pH-dependent anti-CD3 binders display a wide range of activating functions. The bsAb containing anti-CD3 scFv variant 0280 did not activate T cells at pH 7.4 at any concentration tested, consistent with undetectable monovalent binding at pH 7.4. BsAbs containing anti-CD3 scFv variants 0122 and 0094, which display a >70% reduction in cellular binding relative to the wild type antibody at pH 7.4, are capable of fully activating T cells similar to the wildtype construct in a bsAb format. The bsAb containing anti-CD3 variant 0243 displayed about 50% reduction in T cell activation relative to the wild type TCE, and accordingly binds T cells about 50% less than the bsAb containing anti-CD3 scFv variant 0122 at pH 7.4.

Notably, all bsAbs were built in the same scFv-VHH configuration and contained the same tumor target VHH with an affinity of about 2 nM. This suggests CD3 avidity effects, driven by antigen density on the target cell and the relative affinities of the CD3 engager and the target engager determine T cell activation potential.

### Example 6. Anti-CD3 Constructs with a New VL Variant

An additional round of optimization was performed to generate anti-CD3 constructs with anew VL variant. In Round 4 of the optimization, 10 new anti-CD3 variants were selected. These variants contained Q90E, S92D, S93H, P95H, and L96V mutations in the VL domain and showed significantly higher selectivity. Selectivity indices for variants 0436, 0439, 0437, 0438, and 0443 were more than 50. Selectivity indices for variants 0436 and 0439 were more than 90. The most selective variant was 0436 with a selectivity index of 99.11. EC₅₀ and maximum MFI of the variants selected from Round 4 are summarized in Table 4 below.

TABLE 4
Summary of EC₅₀ and maximum MFI
of anti-CD3 constructs (Round 4)

| Construct Name | EC₅₀ pH 6.0 | EC₅₀ pH 7.4 | SI |
|---|---|---|---|
| CD3_scfv_0436 | 1.01E−08 | 1.00E−06 | 99.11 |
| CD3_scfv_0439 | 1.05E−08 | 1.00E−06 | 94.88 |
| CD3_scfv_0437 | 1.21E−08 | 1.00E−06 | 82.58 |
| CD3_scfv_0438 | 1.27E−08 | 1.00E−06 | 78.93 |
| CD3_scfv_0443 | 1.64E−08 | 1.00E−06 | 60.98 |
| CD3_scfv_0435 | 2.04E−08 | 1.00E−06 | 49.14 |
| CD3_scfv_0442 | 2.88E−08 | 1.00E−06 | 34.72 |
| CD3_scfv_0305 | 5.14E−09 | 5.51E−08 | 10.73 |
| CD3_scfv_0434 | 1.08E−08 | 1.08E−08 | 7.59 |
| CD3_scfv_0441 | 7.96E−09 | 5.20E−08 | 6.51 |

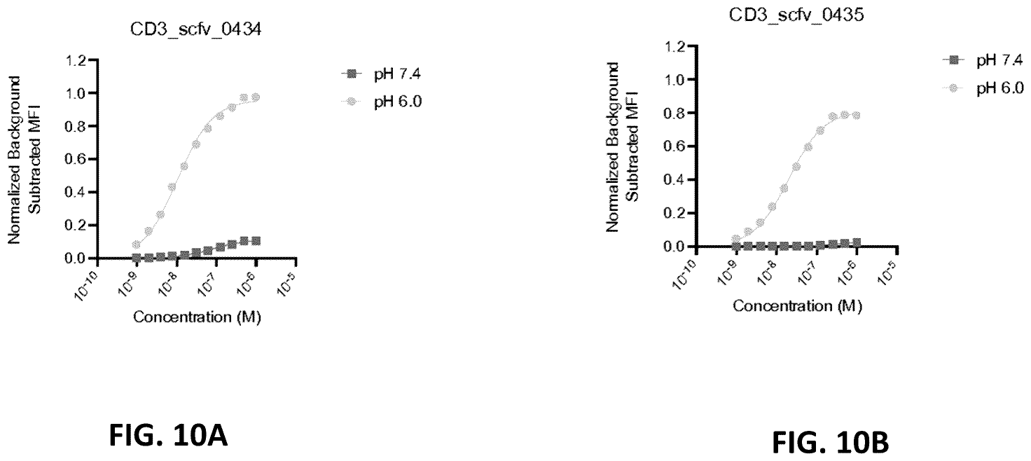

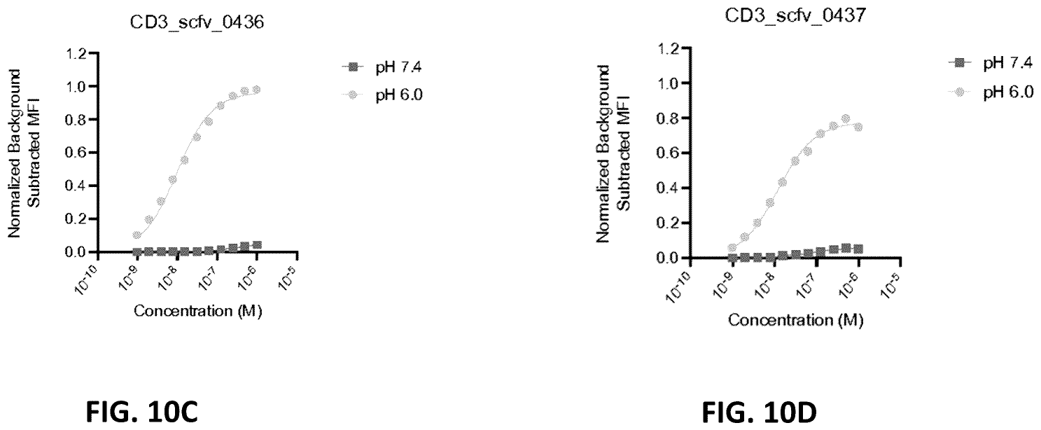

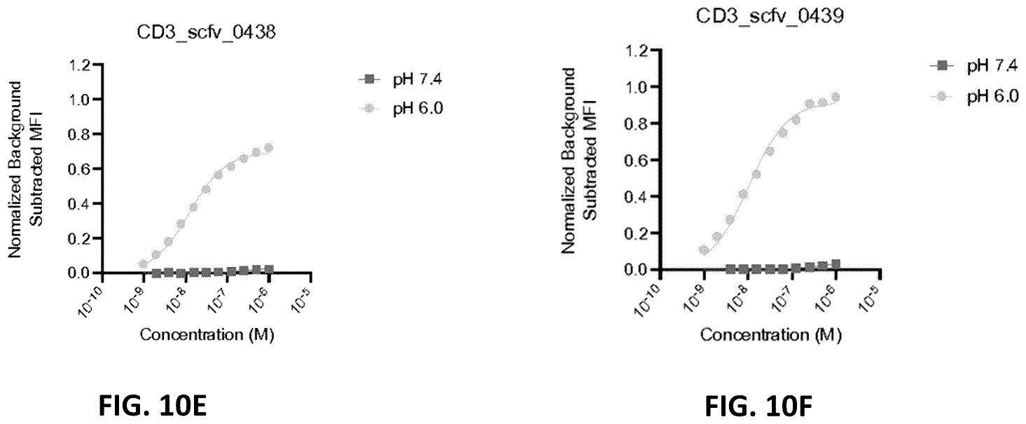

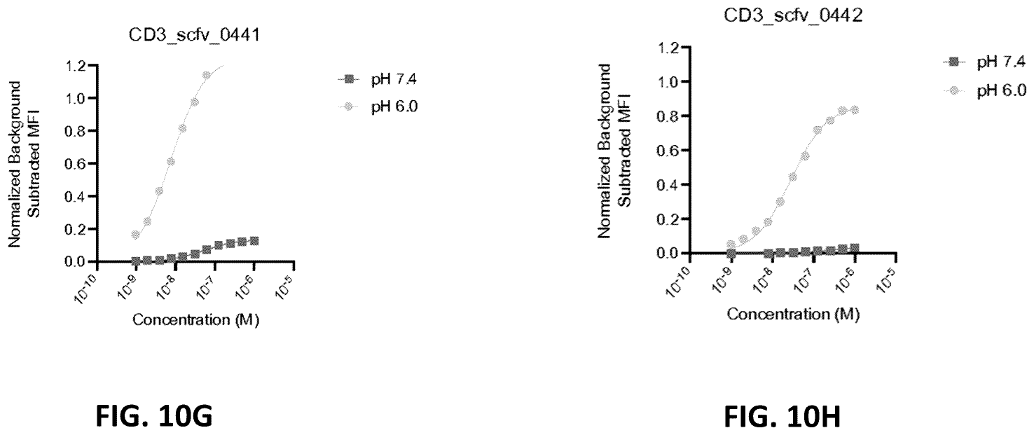

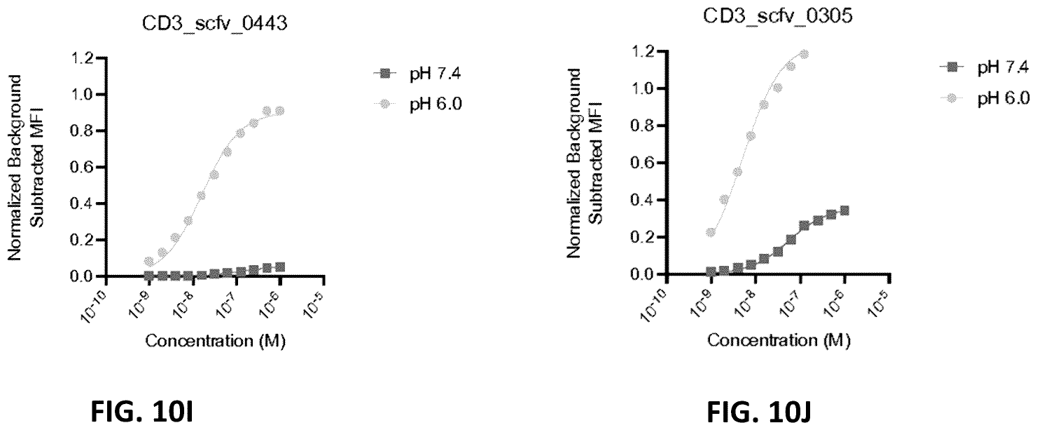

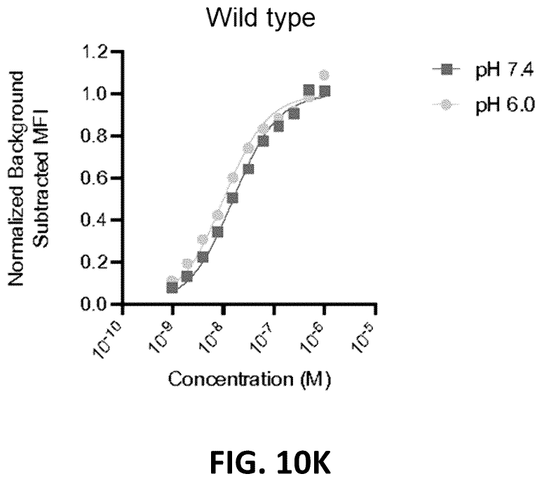

As shown in FIGS. 10A-10K, the anti-CD3 variants selected exhibited significantly increased CD3 binding affinity at pH 6.0 compared to pH 7.4. For variants 0436 (FIG. 10C), 0439 (FIG. 10F), 0437 (FIG. 10D), 0438 (FIG. 10E), and 0443 (FIG. 10I), the binding affinity at pH 6.0 is 50-fold or more greater than the binding affinity at pH 7.4, as shown in Table 4. For variants 0436 (FIG. 10C) and 0439 (FIG. 10F), the binding affinity at pH 6.0 is 90-fold or more greater than the binding affinity at pH 7.4, as shown in Table 4. It is also observed that some of the variants, 0435 (FIG. 10B), 0436 (FIG. 10C), 0437 (FIG. 10D), 0438 (FIG. 10E), 0439 (FIG. 10F), 0442 (FIG. 10H), 0443 (FIG. 10I) displayed very low or undetectable binding at pH 7.4, as shown in Table 4.

This example illustrates that the anti-CD3 variants described herein can better target tumor cells and reduce off-target toxicity in a tumor microenvironment.

### Example 7. Validation of Anti-CD3 Constructs at Various pH

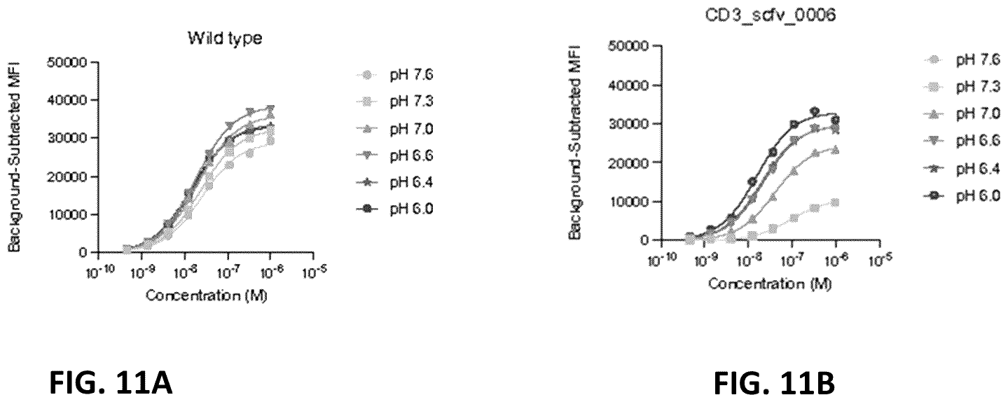

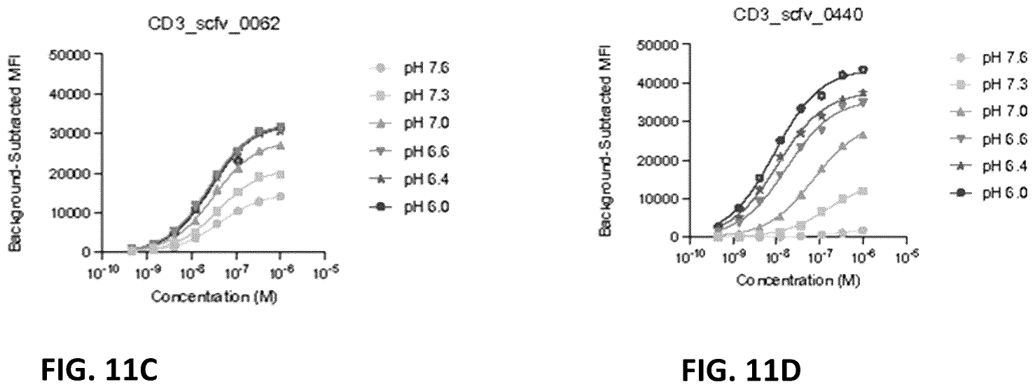

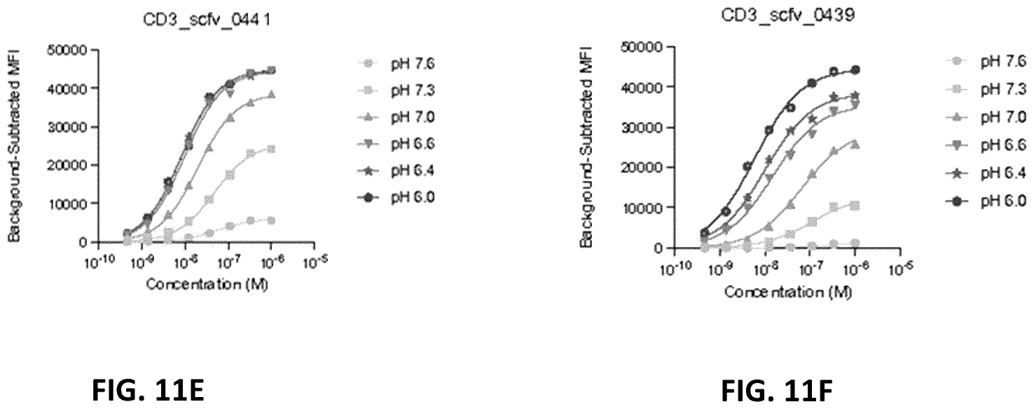

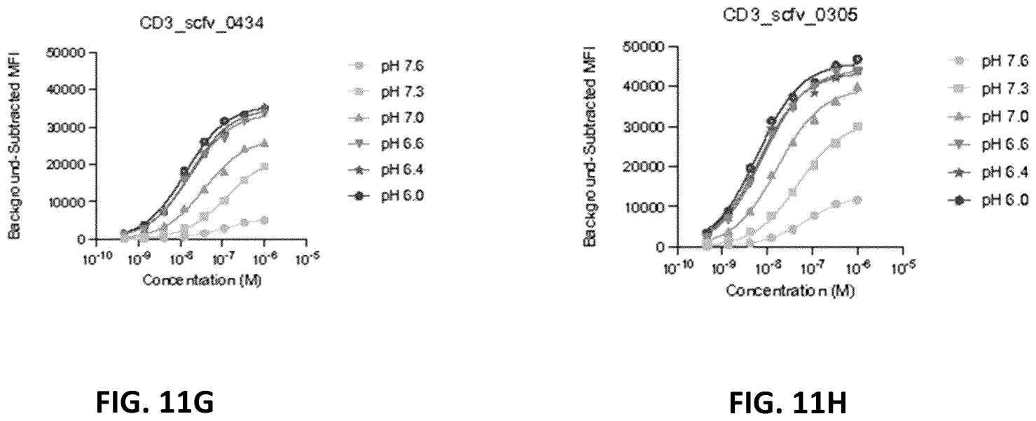

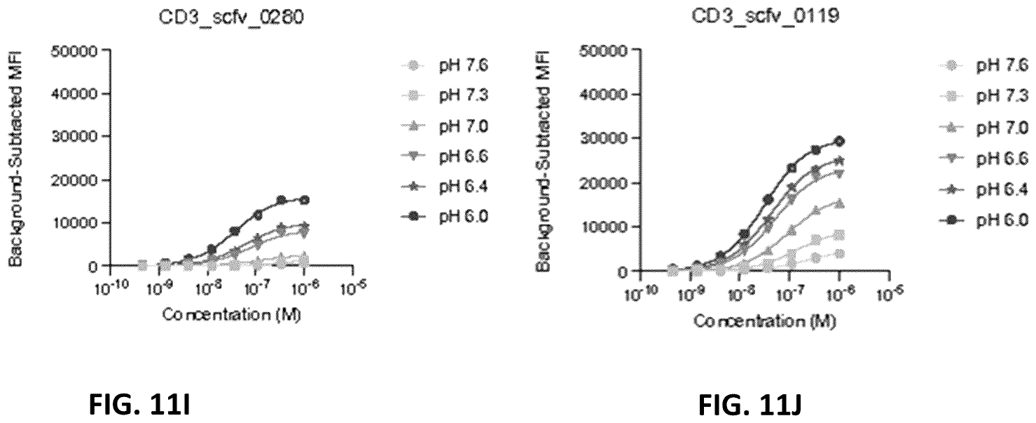

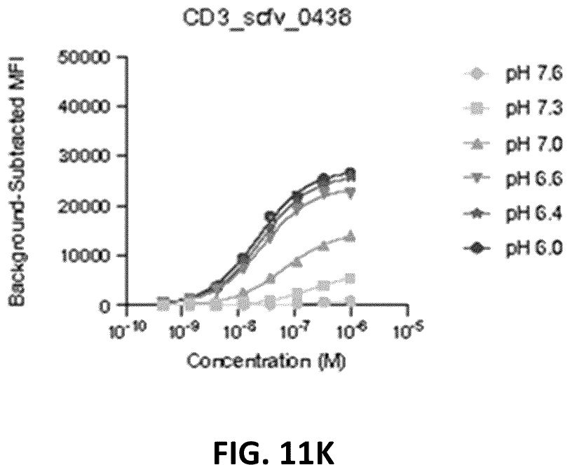

To validate anti-CD3 variants, a wildtype control and several clones utilized a 8 point serial dilution at pH 6.0, 6.4, 6.6, 7.0, 7.3 and 7.6. As shown in FIG. 11B, the anti-CD3 variant 0006 exhibited significantly increased CD3 binding affinity as pH decreased. As shown in FIG. 11C, the anti-CD3 variant 0062 exhibited significantly increased CD3 binding affinity as pH decreased. As shown in FIG. 11D, the anti-CD3 variant 0440 exhibited significantly increased CD3 binding affinity as pH decreased. As shown in FIG. 11E, the anti-CD3 variant 0441 exhibited significantly increased CD3 binding affinity as pH decreased. As shown in FIG. 11F, the anti-CD3 variant 0439 exhibited significantly increased CD3 binding affinity as pH decreased. As shown in FIG. 11G, the anti-CD3 variant 0434 exhibited significantly increased CD3 binding affinity as pH decreased. As shown in FIG. 11H, the anti-CD3 variant 0305 exhibited significantly increased CD3 binding affinity as pH decreased. As shown in FIG. 11I, the anti-CD3 variant 0280 exhibited significantly increased CD3 binding affinity as pH decreased. As shown in FIG. 11J, the anti-CD3 variant 0119 exhibited significantly increased CD3 binding affinity as pH decreased. As shown in FIG. 11K, the anti-CD3 variant 0438 exhibited significantly increased CD3 binding affinity as pH decreased. These results provide clear evidence that the constructs demonstrated a pH dependent binding behavior (FIG. 11A-11K). This example also illustrates that the anti-CD3 variants described herein can better target tumor cells and reduce off-target toxicity in a tumor microenvironment.

### Example 8. Validation of Anti-CD3 Bispecifics with a VHH Targeting a Tumor Antigen

Engineered anti-CD3 scFv variants displaying a range of pH dependent binding were reformatted into bispecifics with a VHH targeting a tumor antigen. These bispecifics were tested for binding by FACS to Jurkat E6.1 T cells at pH 7.4 and pH 6.4 to confirm that reformatting does not impact pH dependent binding using the protocol described previously. T cells were then cocultured with cancer cells expressing the tumor target antigen at a 10:1 ratio (100,000 T cells with 10,000 target cells) and incubated with 200 nM bispecific T cell engager for 72 hours in RPMI-based culture medium either at pH 7.4 or adjusted to pH 6.4. 200 nM was selected as T cell binding was saturated at this concentration for all bispecifics tested. After 72 hours, cell suspensions were removed and stained with anti-CD69-DY647 secondary antibodies (1:200 dilution), washed and analyzed for the fraction of cells expressing CD69 (activated T cells). CD69 levels for each condition were normalized to the CD69 level from the wild type bispecific condition.

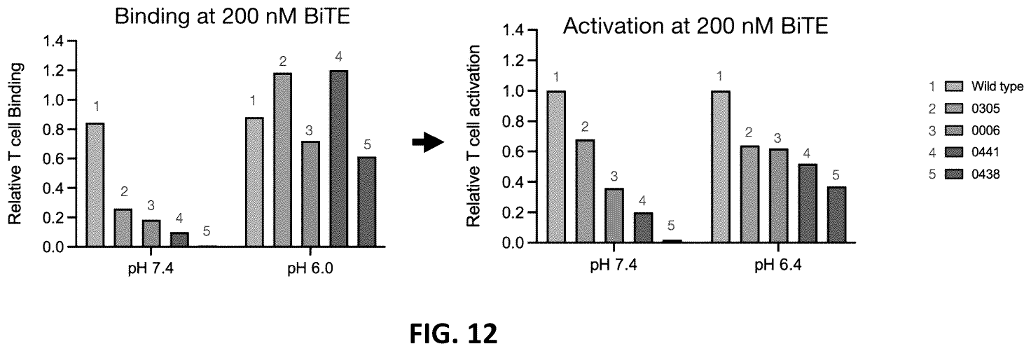

As shown in FIG. 12, these experiments demonstrate that the engineered CD3 agonist scFvs retain their pH dependent binding properties when reformatted into bispecifics.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

SEQUENCES

| | | SEQ ID | |
|---|---|---|---|
| Construct | Description | NQ: | Sequence |
| wild type | Full Length | 1 | DIKLQQSGAELARPGASVKMSCKTSGYTFTRYTMH |
| | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| anti-CD3 | VH Domain | 22 | DIKLQQSGAELARPGASVKMSCKTSGYTFTRYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| scFv | VL Domain | 38 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 47 | RYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 2 | DIKLQQSGAELARPGASVKMSCKTSGYTFTHYTMH |
| 0280 | Sequence | | WVKQRPGQGLEWIGYINPSRGHTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 23 | DIKLQQSGAELARPGASVKMSCKTSGYTFTHYTMH |
| | | | WVKQRPGQGLEWIGYINPSRGHTNYNQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 50 | YINPSRGHTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 3 | DIKLQQSGAELARPGASVKMSCKTSGYTFTRYTMH |
| 0268 | Sequence | | WVKQRPGQGLEWIGYINPSRGYTNYNQKFKDKAT |
| | | | LTTDKSSSTAYMQLSSLTSEDSAVYYCARYTDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWSSNPLTFGAGTKLELK |
| | VH Domain | 24 | DIKLQQSGAELARPGASVKMSCKTSGYTFTRYTMH |
| | | | WVKQRPGQGLEWIGYINPSRGYTNYNQKFKDKAT |
| | | | LTTDKSSSTAYMQLSSLTSEDSAVYYCARYTDDHY |
| | | | CLDYWGQGTTLTVSS |
| | VL Domain | 40 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWSSNPLTFGAGTKLELK |
| | Linker | 46 | VEGGSGGSGGSGGSGGVD |
| | HCDR1 | 47 | RYTMH |
| | HCDR2 | 49 | YINPSRGYTNYNQKFKD |
| | HCDR3 | 61 | YTDDHYCLDY |
| | LCDR1 | 62 | RASSSVSYMN |
| | LCDR2 | 64 | DTSKVAS |
| | LCDR3 | 66 | QEWSSNPLT |
| CD3_scfv_ | Full Length | 4 | DIKLQQSGAELARPGASVKMSCKTSGYTFTHYTMH |
| 0243 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIEDTSKVASGVPYRFSGSGSGTSYSLTI |
| | | | SSMEAEDAATYYCQQWSSNPLTFGAGTKLELK |
| | VH Domain | 25 | DIKLQQSGAELARPGASVKMSCKTSGYTFTHYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 41 | DIQLTQSPAIMSASPGEKVTMTCRASSSVSYMNWY |
| | | | QQKSGTSPKRWIEDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 5 | DIKLQQSGAELARPGASVKMSCKTSGYTFTHYTMH |
| 0291 | Sequence | | WVKQRPGQGLEWIGYINPSRGYTNENQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 26 | DIKLQQSGAELARPGASVKMSCKTSGYTFTHYTMH |
| | | | WVKQRPGQGLEWIGYINPSRGYTNENQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 51 | YINPSRGYTNENQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 6 | DIKLQQSGAELARPGASVKMSCKTSGYTHTHYTM |
| 0250 | Sequence | | HWVKQRPGQGLEWIGYINPSRGYTNYNQKFKDKA |
| | | | TLTTDKSSSTAYMQLSSLTSEDSAVYYCARYYDDH |
| | | | YCLDYWGQGTTLTVSSVEGGSGGSGGSGGSGGVD |
| | | | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | VH Domain | 27 | DIKLQQSGAELARPGASVKMSCKTSGYTHTHYTM |
| | | | HWVKQRPGQGLEWIGYINPSRGYTNYNQKFKDKA |
| | | | TLTTDKSSSTAYMQLSSLTSEDSAVYYCARYYDDH |
| | | | YCLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 7 | DIKLQQSGAELARPGASVKMSCKTSGYTFTHYTMH |
| 0119 | Sequence | | WVKQRPGQGLEWIGYINPSRHYTNHNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 28 | DIKLQQSGAELARPGASVKMSCKTSGYTFTHYTMH |
| | | | WVKQRPGQGLEWIGYINPSRHYTNHNQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 52 | YINPSRHYTNHNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 8 | DIKLQQSGAELARPGASVKMSCKTSGYTFTHYTMH |
| 0224 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCHQWSSNPLTFGAGTKLELK |
| | VH Domain | 25 | DIKLQQSGAELARPGASVKMSCKTSGYTFTHYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 42 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCHQWSSNPLTFGAGTKLELK |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 67 | HQWSSNPLT |
| CD3_scfv_ | Full Length | 9 | DIKLQQSGAELARPGASVKMSCKTSGYTFTHYTMH |
| 0247 | Sequence | | WVKQRPGQGLEWIGYINPSRGYTNHNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 29 | DIKLQQSGAELARPGASVKMSCKTSGYTFTHYTMH |
| | | | WVKQRPGQGLEWIGYINPSRGYTNHNQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 53 | YINPSRGYTNHNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3 scfv_ | Full Length | 10 | DIKLQQSGAELARPGASVKMSCKTSGYTFTHYTMH |
| 0235 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQHWSSNPLTFGAGTKLELK |
| | VH Domain | 25 | DIKLQQSGAELARPGASVKMSCKTSGYTFTHYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 43 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQHWSSNPLTFGAGTKLELK |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 68 | QHWSSNPLT |
| CD3_scfv_ | Full Length | 11 | DIKLQQSGAELARPGASVKMSCKTSGYTFTHYTMH |
| 0104 | Sequence | | WVKQRPGQGLEWIGYINPSRGYTNYNQKVKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 30 | DIKLQQSGAELARPGASVKMSCKTSGYTFTHYTMH |
| | | | WVKQRPGQGLEWIGYINPSRGYTNYNQKVKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 54 | YINPSRGYTNYNQKVKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 12 | DIKLQQSGAELARPGASVKMSCKTSGYTFTHYTMH |
| 0241 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSHPLTFGAGTKLELK |
| | VH Domain | 25 | DIKLQQSGAELARPGASVKMSCKTSGYTFTHYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 44 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSHPLTFGAGTKLELK |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 69 | QQWSSHPLT |
| CD3_scfv_ | Full Length | 13 | DIKLQQSGAELARPGASVKMSCKTSGYTFTRYTMH |
| 0233 | Sequence | | WVKQRPGQGLEWIGYINPSRGYTNENQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVHYMNWYQ |
| | | | QKSGTSPKRWIYDTSKVASGVPYRFSGSGSGTSYSL |
| | | | TISSMEAEDAATYYCQQWSSNPLTFGAGTKLELK |
| | VH Domain | 31 | DIKLQQSGAELARPGASVKMSCKTSGYTFTRYTMH |
| | | | WVKQRPGQGLEWIGYINPSRGYTNENQKFKDKAT |
| | | | LTTDKSSSTAYMQLSSLTSEDSAVYYCARYYDDHY |
| | | | CLDYWGQGTTLTVSS |
| | VL Domain | 45 | DIQLTQSPAIMSASPGEKVTMTCRASSSVHYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 47 | RYTMH |
| | HCDR2 | 51 | YINPSRGYTNENQKFKD |
| | HCDR3 | 60 | YYDDHYCLDY |
| | LCDR1 | 63 | RASSSVHYMN |
| | LCDR2 | 64 | DTSKVAS |
| | LCDR3 | 65 | QQWSSNPLT |
| CD3_scfv_ | Full Length | 14 | DIKLQQSGAELARPGASVKMSCKTSGYTFTHYTMH |
| 0261 | Sequence | | WVKQRPGQGLEWIGYINPSRGYTNYHQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 32 | DIKLQQSGAELARPGASVKMSCKTSGYTFTHYTMH |
| | | | WVKQRPGQGLEWIGYINPSRGYTNYHQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 55 | YINPSRGYTNYHQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 15 | DIKLQQSGAELARPGASVKMSCKTSGYTFTRYTMH |
| 0293 | Sequence | | WVKQRPGQGLEWIGYINPSRGYHNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIEDTSKVASGVPYRFSGSGSGTSYSLTI |
| | | | SSMEAEDAATYYCQQWSSNPLTFGAGTKLELK |
| | VH Domain | 33 | DIKLQQSGAELARPGASVKMSCKTSGYTFTRYTMH |
| | | | WVKQRPGQGLEWIGYINPSRGYHNYNQKFKDKAT |
| | | | LTTDKSSSTAYMQLSSLTSEDSAVYYCARYYDDHY |
| | | | CLDYWGQGTTLTVSS |
| | VL Domain | 41 | DIQLTQSPAIMSASPGEKVTMTCRASSSVSYMNWY |
| | | | QQKSGTSPKRWIEDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 47 | RYTMH |
| | HCDR2 | 56 | YINPSRGYHNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 16 | DIKLQQSGAELARPGASVKMSCKTSGYTFTRYTMH |
| 0060 | Sequence | | WVKQRPGQGLEWIGYINPSRGYENYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 34 | DIKLQQSGAELARPGASVKMSCKTSGYTFTRYTMH |
| | | | WVKQRPGQGLEWIGYINPSRGYENYNQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 47 | RYTMH |
| | HCDR2 | 57 | YINPSRGYENYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 17 | DIKLQQSGAELARPGASVKMSCKTSGYTFTHYTMH |
| 0094 | Sequence | | WVKQRPGQGLEWIGYINPSRGYTHYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 35 | DIKLQQSGAELARPGASVKMSCKTSGYTFTHYTMH |
| | | | WVKQRPGQGLEWIGYINPSRGYTHYNQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 58 | YINPSRGYTHYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 18 | DIKLQQSGAELARPGASVKMSCKTSGYTFTHYTMH |
| 0122 | Sequence | | WVKQRPGQGLEWIGYINPHRGYTNYNHKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 36 | DIKLQQSGAELARPGASVKMSCKTSGYTFTHYTMH |
| | | | WVKQRPGQGLEWIGYINPHRGYTNYNHKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 59 | YINPHRGYTNYNHKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 19 | DIKLQQSGAELARPGASVKMSCKTSGYTFTHYTMH |
| 0006 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQQWSSNPLTFGAGTKLELK |
| | VH Domain | 25 | DIKLQQSGAELARPGASVKMSCKTSGYTFTHYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 20 | DIKLQQSGAELARPGASVKMSCKTSGYTFTRYTMH |
| 0287 | Sequence | | WVKQRPGQGLEWIGYINPSRGYHNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWSSNPLTFGAGTKLELK |
| | VH Domain | 33 | DIKLQQSGAELARPGASVKMSCKTSGYTFTRYTMH |
| | | | WVKQRPGQGLEWIGYINPSRGYHNYNQKFKDKAT |
| | | | LTTDKSSSTAYMQLSSLTSEDSAVYYCARYYDDHY |
| | | | CLDYWGQGTTLTVSS |
| | VL Domain | 40 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWSSNPLTFGAGTKLELK |
| | Linker | 46 | VEGGSGGSGGSGGSGGVD |
| | HCDR1 | 47 | RYTMH |
| | HCDR2 | 56 | YINPSRGYHNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 66 | QEWSSNPLT |
| CD3_scfv_ | Full Length | 21 | DIKLQQSGAELARPGASVKMSCKTSGYTFWRYTM |
| 0099 | Sequence | | HWVKQRPGQGLEWIGYINPSRGYTNYNQKFKDKA |
| | | | TLTTDKSSSTAYMQLSSLTSEDSAVYYCARYTDDH |
| | | | YCLDYWGQGTTLTVSSVEGGSGGSGGSGGSGGVD |
| | | | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | VH Domain | 37 | DIKLQQSGAELARPGASVKMSCKTSGYTFWRYTM |
| | | | HWVKQRPGQGLEWIGYINPSRGYTNYNQKFKDKA |
| | | | TLTTDKSSSTAYMQLSSLTSEDSAVYYCARYTDDH |
| | | | YCLDYWGQGTTLTVSS |
| | VL Domain | 39 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQQWSSNPLTFGAGTKLELK |
| | Linker | 46 | VEGGSGGSGGSGGSGGVD |
| | HCDR1 | 47 | RYTMH |
| | HCDR2 | 49 | YINPSRGYTNYNQKFKD |
| | HCDR3 | 61 | YTDDHYCLDY |
| | LCDR1 | 62 | RASSSVSYMN |
| | LCDR2 | 64 | DTSKVAS |
| | LCDR3 | 65 | QQWSSNPLT |
| CD3_scfv_ | Full Length | 605 | DIKLQQSGAELARPGASVKMSCKTSGYTFTHYTMH |
| 0436 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 25 | DIKLQQSGAELARPGASVKMSCKTSGYTFTHYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 48 | HYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 606 | DIKLQQSGAELARPGASVKMSCKTSGYTFTHYTMH |
| 0439 | Sequence | | WVKQRPGQGLEWIGYINPSRGYTNYNHKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 615 | DIKLQQSGAELARPGASVKMSCKTSGYTFTHYTMH |
| | | | WVKQRPGQGLEWIGYINPSRGYTNYNHKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 48 | HYTMH |
| | HCDR2 | 529 | YINPSRGYTNYNHKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 607 | DIKLQQSGAELARPGASVKMSCKTSGYTFTRYTMH |
| 0437 | Sequence | | WVKQRPGQGLEWIGYINPSRGYTNYNQKVKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 359 | DIKLQQSGAELARPGASVKMSCKTSGYTFTRYTMH |
| | | | WVKQRPGQGLEWIGYINPSRGYTNYNQKVKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 54 | YINPSRGYTNYNQKVKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 608 | DIKLQQSGAELARPGASVKMSCKTSGYTFTRYTMH |
| 0438 | Sequence | | WVKQRPGQGLEWIGYINPSRGYTNENQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 31 | DIKLQQSGAELARPGASVKMSCKTSGYTFTRYTMH |
| | | | WVKQRPGQGLEWIGYINPSRGYTNENQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 51 | YINPSRGYTNENQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 609 | DIKLQQSGAELARPGASVKMSCKTSGYTHTRYTMH |
| 0443 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 318 | DIKLQQSGAELARPGASVKMSCKTSGYTHTRYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 610 | DIKLQQSGAELARPGASVKMSCKTSGYTFTHYTMH |
| 0435 | Sequence | | WVKQRPGQGLEWIGYINPSRHYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 616 | DIKLQQSGAELARPGASVKMSCKTSGYTFTHYTMH |
| | | | WVKQRPGQGLEWIGYINPSRHYTNYNQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 48 | HYTMH |
| | HCDR2 | 516 | YINPSRHYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 611 | DIKLQQSGAELARPGASVKMSCKTSGYTFTRYTMH |
| 0442 | Sequence | | WVKQRPGQGLEWIGYINPSRGHTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 368 | DIKLQQSGAELARPGASVKMSCKTSGYTFTRYTMH |
| | | | WVKQRPGQGLEWIGYINPSRGHTNYNQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 50 | YINPSRGHTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 612 | DIKLQQSGAELARPGASVKMSCKTSGYTFTRYTMH |
| 0305 | Sequence | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 22 | DIKLQQSGAELARPGASVKMSCKTSGYTFTRYTMH |
| | | | WVKQRPGQGLEWIGYINPSRGYTNYNQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 49 | YINPSRGYTNYNQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 613 | DIKLQQSGAELARPGASVKMSCKTSGYTFTRYTMH |
| 0434 | Sequence | | WVKQRPGQGLEWIGYINPSRGYTNHNQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 322 | DIKLQQSGAELARPGASVKMSCKTSGYTFTRYTMH |
| | | | WVKQRPGQGLEWIGYINPSRGYTNHNQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 53 | YINPSRGYTNHNQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |
| CD3_scfv_ | Full Length | 614 | DIKLQQSGAELARPGASVKMSCKTSGYTFTRYTMH |
| 0441 | Sequence | | WVKQRPGQGLEWIGYINPSRGYTNYHQKFKDKAT |
| | | | LTTDKSSSTAYMQLSSLTSEDSAVYYCARYYDDHY |
| | | | CLDYWGQGTTLTVSSVEGGSGGSGGSGGSGGVDDI |
| | | | QLTQSPAIMSASPGEKVTMTCRASSSVSYMNWYQQ |
| | | | KSGTSPKRWIYDTSKVASGVPYRFSGSGSGTSYSLT |
| | | | ISSMEAEDAATYYCQEWDHNHVTFGAGTKLELK |
| | VH Domain | 316 | DIKLQQSGAELARPGASVKMSCKTSGYTFTRYTMH |
| | | | WVKQRPGQGLEWIGYINPSRGYTNYHQKFKDKAT |
| | | | LTTDKSSSTAYMQLSSLTSEDSAVYYCARYYDDHY |
| | | | CLDYWGQGTTLTVSS |
| | VL Domain | 617 | DIQLTQSPAIMSASPGEKVTMTCRASSSVSYMNWY |
| | | | QQKSGTSPKRWIYDTSKVASGVPYRFSGSGSGTSYS |
| | | | LTISSMEAEDAATYYCQEWDHNHVTFGAGTKLEL |
| | | | K |
| | Linker | 46 | VEGGSGGSGGSGGSGGVD |
| | HCDR1 | 47 | RYTMH |
| | HCDR2 | 55 | YINPSRGYTNYHQKFKD |
| | HCDR3 | 60 | YYDDHYCLDY |
| | LCDR1 | 62 | RASSSVSYMN |
| | LCDR2 | 64 | DTSKVAS |
| | LCDR3 | 618 | QEWDHNHVT |

| Sequence | SEQ ID NO: |
|---|---|
| X₁YTMH | 70 |
| X₂INPSRX₃X₄TNX₅NQKFKD | 71 |
| YX₆DDHYCLDY | 72 |
| QX₇WSSNPLT | 73 |
| X₈INPX₉RX₁₀X₁₁X₁₂X₁₃X₁₄X₁₅X₁₆KX₁₇KD | 74 |
| RASSSVX₁₈YMN | 75 |
| X₁₉X₂₀WSSX₂₁PLT | 76 |
| EINPSRX₃X₄TNX₅NQKFKD | 77 |
| X₂INPSRHX₄TNX₅NQKFKD | 78 |
| X₂INPSRX3HTNX₅NQKFKD | 79 |
| EINPX₉RX₁₀X₁₁X₁₂X₁₃X₁₄X₁₅X₁₆KX₁₇KD | 80 |
| X₈INPRRX₁₀X₁₁X₁₂X₁₃X₁₄X₁₅X₁₆KX₁₇KD | 81 |
| X₈INPX₉RHX₁₁X₁₂X₁₃X₁₄X₁₅X₁₆KX₁₇KD | 82 |
| X₈INPX₉RX₁₀HX₁₂X₁₃X₁₄X₁₅X₁₆KX₁₇KD | 83 |
| X₈INPX₉RX₁₀X₁₁X₁₂HX₁₄X₁₅X₁₆KX₁₇KD | 84 |
| X₈INPX₉RX₁₀X₁₁X₁₂X₁₃X₁₄HX₁₆KX₁₇KD | 85 |
| X₈INPX₉RX₁₀X₁₁X₁₂X₁₃X₁₄X₁₅HKX₁₇KD | 86 |
| X₈INPX₉RX₁₀X₁₁X₁₂X₁₃X₁₄X₁₅X₁₆KVKD | 87 |
| HX₂₀WSSX₂₁PLT | 88 |
| X₁₉EWSSX₂₁PLT | 89 |
| X₁₉X₂₀WSSHPLT | 90 |

1. An anti-CD3 antibody or antigen-binding fragment thereof comprising (a) a HCDR1 having the amino acid sequence set out in any one of SEQ ID NOs: 47-48, (b) a HCDR2 having the amino acid sequence set out in any one of SEQ ID NOs: 49-59, and (c) a HCDR3 having the amino acid sequence set out in any one of SEQ ID NOs: 60-61, wherein the anti-CD3 antibody or antigen-binding fragment thereof has a binding affinity for CD3 that is 10-fold or more greater at an acidic pH than at a non-acidic pH.

2.-3. (canceled)

4. The anti-CD3 antibody or antigen-binding fragment thereof of claim 1, further comprising (a) a LCDR1 having the amino acid sequence set out in any one of SEQ ID NOs: 62-63, (b) a LCDR2 having the amino acid sequence set out in SEQ ID NO: 64, and (c) a LCDR3 having the amino acid sequence set out in any one of SEQ ID NOs: 65-69 or 618.

5.-7. (canceled)

8. The anti-CD3 antibody or antigen-binding fragment thereof of claim 1, wherein the anti-CD3 antibody or antigen-binding fragment thereof comprises a heavy chain variable domain having at least 90% sequence identity to the amino acid sequence set out in any one of SEQ ID NOs: 23-37, 615 or 616.

9. (canceled)

10. The anti-CD3 antibody or antigen-binding fragment thereof of claim 1, wherein the anti-CD3 antibody or antigen-binding fragment thereof comprises a light chain variable domain having at least 90% sequence identity to the amino acid sequence set out in any one of SEQ ID NOs: 39-45 or 617.

11.-14. (canceled)

15. An anti-CD3 antibody or antigen-binding fragment thereof comprising: (i) a HCDR1 as set out in SEQ ID NO: 48, a HCDR2 as set out in SEQ ID NO: 50, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 65; (ii) a HCDR1 as set out in SEQ ID NO: 47, a HCDR2 as set out in SEQ ID NO: 49, a HCDR3 as set out in SEQ ID NO: 61, a LCDR1 as set out in SEQ ID NO: 62, a LCDR2 as set out in SEQ ID NO: 64, and a LCDR3 as set out in SEQ ID NO: 66; (iii) a HCDR1 as set out in SEQ ID NO: 48, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 65; (iv) a HCDR1 as set out in SEQ ID NO: 48, a HCDR2 as set out in SEQ ID NO: 51, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 65; (v) a HCDR1 as set out in SEQ ID NO: 48, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 65; (vi) a HCDR1 as set out in SEQ ID NO: 48, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 61; (vii) a HCDR1 as set out in SEQ ID NO: 48, a HCDR2 as set out in SEQ ID NO: 529, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (viii) a HCDR1 as set out in SEQ ID NO: 47, a HCDR2 as set out in SEQ ID NO: 54, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (ix) a HCDR1 as set out in SEQ ID NO: 47, a HCDR2 as set out in SEQ ID NO: 51, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (x) a HCDR1 as set out in SEQ ID NO: 47, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (xi) a HCDR1 as set out in SEQ ID NO: 48, a HCDR2 as set out in SEQ ID NO: 516, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (xii) a HCDR1 as set out in SEQ ID NO: 47, a HCDR2 as set out in SEQ ID NO: 50, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (xiii) a HCDR1 as set out in SEQ ID NO: 47, a HCDR2 as set out in SEQ ID NO: 49, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; (xiv) a HCDR1 as set out in SEQ ID NO: 47, a HCDR2 as set out in SEQ ID NO: 53, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618; or (xv) a HCDR1 as set out in SEQ ID NO: 47, a HCDR2 as set out in SEQ ID NO: 55, a HCDR3 as set out in SEQ ID NO: 60, a LCDR1 as set out in SEQ ID NO: 62, a LCDR2 as set out in SEQ ID NO: 64, and a LCDR3 as set out in SEQ ID NO: 618.

16.-66. (canceled)

67. An anti-CD3 antibody or antigen-binding fragment thereof comprising: (i)(a) a HCDR1 having an amino acid sequence HYTMH (SEQ ID NO: 48); (b) a HCDR2 having an amino acid sequence X₂INPSRX₃X₄TNX₅NQKFKD (SEQ ID NO: 71), wherein X₂ is Y or E, X₃ is G or H, X₄ is Y or H, and X₅ is Y, E, or H; and (c) a HCDR3 having an amino acid sequence YX₆DDHYCLDY (SEQ ID NO: 72), wherein X₆ is Y or T; (ii) (a) a HCDR1 having an amino acid sequence HYTMH (SEQ ID NO: 48); (b) a HCDR2 having an amino acid sequence X₈INPX₉RX₁₀X₁₁X₁₂X₁₃X₁₄X₁₅X₁₆KX₁₇KD (SEQ ID NO: 74), wherein X₈ is Y or E, X₉ is S or R, X₁₀ is G or H, X₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (iii) (a) a HCDR1 having an amino acid sequence X₁YTMH (SEQ ID NO: 70), wherein X₁ is R or H; (b) a HCDR2 having an amino acid sequence EINPSRX₃X₄TNX₅NQKFKD (SEQ ID NO: 77), wherein X₃ is G or H, X₄ is Y or H, and X₅ is Y, E, or H; and (c) a HCDR3 having an amino acid sequence YX₆DDHYCLDY (SEQ ID NO: 72), wherein X₆ is Y or T; (iv) (a) a HCDR1 having an amino acid sequence X₁YTMH (SEQ ID NO: 70), wherein X₁ is R or H; (b) a HCDR2 having an amino acid sequence X₂INPSRHX₄TNX₅NQKFKD (SEQ ID NO: 78), wherein X₂ is Y or E, X₄ is Y or H, and X₅ is Y, E, or H; and (c) a HCDR3 having an amino acid sequence YX₆DDHYCLDY (SEQ ID NO: 72), wherein X₆ is Y or T; (v) (a) a HCDR1 having an amino acid sequence X₁YTMH (SEQ ID NO: 70), wherein X₁ is R or H; (b) a HCDR2 having an amino acid sequence X₂INPSRX₃HTNX₅NQKFKD (SEQ ID NO: 79), wherein X₂ is Y or E, X₃ is G or H, and X₅ is Y, E, or H; and (c) a HCDR3 having an amino acid sequence YX₆DDHYCLDY (SEQ ID NO: 72), wherein X₆ is Y or T; (vi) (a) a HCDR1 having an amino acid sequence X₁YTMH (SEQ ID NO: 70), wherein X₁ is R or H; (b) a HCDR2 having an amino acid sequence X₂INPSRX₃X₄TNX₅NQKFKD (SEQ ID NO: 71), wherein X₂ is Y or E, X₃ is G or H, X₄ is Y or H, and X₅ is E, or H; and (c) a HCDR3 having an amino acid sequence YX₆DDHYCLDY (SEQ ID NO: 72), wherein X₆ is Y or T; (vii)(a) a HCDR1 having an amino acid sequence X₁YTMH (SEQ ID NO: 70), wherein X₁ is R or H; (b) a HCDR2 having an amino acid sequence EINPX₉RX₁₀X₁₁X₁₂X₁₃X₁₄X₁₅X₁₆KX₁₇KD (SEQ ID NO: 80), X₉ is S or R, X₁₀ is G or H, X₁₁ is Y or H, X₁₂ is T, H, or E, X₁₃ is N or H, X₁₄ is Y, E, or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (viii)(a) a HCDR1 having an amino acid sequence X₁YTMH (SEQ ID NO: 70), wherein X₁ is R or H; (b) a HCDR2 having an amino acid sequence X₈INPRRX₁₀X₁₁X₁₂X₁₃X₁₄X₁₅X₁₆KX₁₇KD (SEQ ID NO: 81), wherein X₈ is Y or E, X₁₀ is G or H, X₁ is Y or H, X₁₂ is T, H, or E, X₁₃ is N or H, X₁₄ is Y, E, or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (ix) (a) a HCDR1 having an amino acid sequence X₁YTMH (SEQ ID NO: 70), wherein X₁ is R or H; (b) a HCDR2 having an amino acid sequence X₈INPX₉RHX₁₁X₁₂X₁₃X₁₄X₁₅X₁₆KX₁₇KD (SEQ ID NO: 82), wherein X₈ is Y or E, X₉ is S or R, X₁₁ is Y or H, X₁₂ is T, H, or E, X₁₃ is N or H, X₁₄ is Y, E, or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (x) (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅ is X₁₆KX₁₇KD (SEQ ID NO: 83), wherein X₈ is Y or E, X₉ is S or R, X₁₀ is G or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (xi) (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KX₁₇KD (SEQ ID NO: 603), wherein X₈ is Y or E, X₉ is S or R, X₁₀ is G or H, X₁₁ is Y or H, X₁₂ is H or E, X₁₃ is N or H, X₁₄ is Y, E, or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (xii) (a) a HCDR1 having an amino acid sequence X₁YTNM (SEQ ID NO: 70), wherein X₁ is R or H; (b) a HCDR2 having an amino acid sequence X₈INPX₉RX₁₀X₁₁X₁₂HX₁₄X₁₅X₁₆KX₁₇KD (SEQ ID NO: 84), wherein X₈ is Y or E, X₉ is S or R, X₁₀ is G or H, X₁₁ is Y or H, X₂ is T, H, or E, X₁₄ is Y, E, or H, X is or H, X₁₆ is Q or H, and X₁₇ is F or V; and (c) a HCDR3 having an amino acid sequence YX₆DDHYCLDY (SEQ ID NO: 72), wherein X₆ is Y or T; (xiii)(a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KX₁₇KD (SEQ ID NO: 604), wherein X₈ is Y or E, X₉ is S or R, X₁₀ is G or H, X₁ is Y or H, X₁₂ is T, H, or E, X₁₃ is N or H, X₁₄ is E or H, X₅ is N or H, X₁₆ is Q or H, and X₁₇ is F or V; and (c) a HCDR3 having an amino acid sequence YX₆DDHYCLDY (SEQ ID NO: 72), wherein X₆ is Y or T; (xiv)(a) a HCDR1 having an amino acid sequence X₁YTNM (SEQ ID NO: 70), wherein X₁ is R or H; (b) a HCDR2 having an amino acid sequence X₈INPX₉RX₁₀X₁₁X₁₂X₁₃X₁₄HX₁₆KX₁₇KD (SEQ ID NO: 85), wherein X₈ is Y or E, X₉ is S or R, X₁₀ is G or H, X₁₁ is Y or H, X₁₂ is T, H, or E, X₁₃ is N or H, X₁₄ is Y, E, or H, X₁₆ is Q or H, and X₁₇ is F or V; and (c) a HCDR3 having an amino acid sequence YX₆DDHYCLDY (SEQ ID NO: 72), wherein X₆ is Y or T; (xv)(a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅HKX₁₇KD (SEQ ID NO: 86), wherein X₈ is Y or E, X₉ is S or R, X₁₀ is G or H, X₁₁ is Y or H, X₁₂ is T, H, or E, X₁₃ is N or H, X₁₄ is Y, E, or H, X₁₅ is N or H, and X₁₇ is F or V; and (c) a HCDR3 having an amino acid sequence YX₆DDHYCLDY (SEQ ID NO: 72), wherein X₆ is Y or T; (xvi)(a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KVKD (SEQ ID NO: 87), wherein X₈ is Y or E, X₉ is S or R, X₁₀ is G or H, X₁₁ is Y or H, X₁₂ is T, H, or E, X₃ is or H, X₁₄ is Y, E, or H, X₁₅ is N or H, and X₁₆ is Q or H; and (c) a HCDR3 having an amino acid sequence YX₆DDHYCLDY (SEQ ID NO: 72), wherein X₆ is Y or T; (xvii) (a) a HCDR1 having an amino acid sequence X₁YTMH (SEQ ID NO: 70), wherein X₁ is R or H; (b) a HCDR2 having an amino acid sequence X₂INPSRX₃X₄TNX₅NQKFKD (SEQ ID NO: 71), wherein X₂ is Y or E, X₃ is G or H, X₄ is Y or H, X₅ is Y, E, or H; and (c) a HCDR3 having an amino acid sequence YTDDHYCLDY (SEQ ID NO: 61): or (xviii) (a) a HCDR1 having an amino acid sequence X₁YTMH (SEQ ID NO: 70), wherein X₁ is R or H; (b) a HCDR2 having an amino acid sequence X₈INPX₉RX₁₀X₁₁X₁₂X₁₃X₁₄X₁₅X₁₆KX₁₇KD (SEQ ID NO: 74), wherein X₈ is Y or E, X₉ is S or R, X₁₀ is G or H, X₁₁ is Y or H, X₁₂ is T, H, or E, X₁₃ is N or H, X₁₄ is Y, E, or H, X₁₅ is N or H, X₁₆ is Q or H, and X₁₇ is F or V; and (c) a HCDR3 having an amino acid sequence YTDDHYCLDY (SEQ ID NO: 61), wherein the anti-CD3 antibody or antigen-binding fragment thereof has a binding affinity for CD3 that is 10-fold or more greater at an acidic pH than at a non-acidic pH.

68.-87. (canceled)

88. The anti-CD3 antibody or antigen-binding fragment thereof of claim 67, further comprising: (d) a LCDR1 having an amino acid sequence RASSSVX₁₈YMN (SEQ ID NO: 75), wherein X₁₈ is S or H; (e) a LCDR2 having an amino acid sequence DTSKVAS (SEQ ID NO: 64); and (f) a LCDR3 having an amino acid sequence HX₂₀WSSX₂₁PLT (SEQ ID NO: 88), wherein X₂₀ is Q or E, and X₂₁ is N or H, or a LCDR3 having an amino acid sequence X₁₉EWSSX₂₁PLT (SEQ ID NO: 89), wherein X₁₉ is Q or H, and X₂₁ is N or H, or a LCDR3 having an amino acid sequence X₁₉X₂₀WSSHPLT (SEQ ID NO: 90), wherein X₁₉ is Q or H, and X₂₀ is Q or E.

89.-106. (canceled)

107. The anti-CD3 antibody or antigen-binding fragment thereof of claim 1, wherein the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fv (scFv) antibody, a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof.

108. The anti-CD3 antibody or antigen-binding fragment thereof of claim 107, wherein the anti-CD3 antibody or antigen-binding fragment thereof is an scFv antibody.

109. The anti-CD3 antibody or antigen-binding fragment thereof of claim 107, wherein the anti-CD3 antibody or antigen-binding fragment thereof is a VHH antibody.

110.-132. (canceled)

133. The anti-CD3 antibody or antigen-binding fragment thereof of claim 15, wherein the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fv (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof.

134. The anti-CD3 antibody or antigen-binding fragment thereof of claim 133, wherein the anti-CD3 antibody or antigen-binding fragment thereof is an scFv antibody.

135. The anti-CD3 antibody or antigen-binding fragment thereof of claim 67, wherein the anti-CD3 antibody or antigen-binding fragment thereof is a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fv (scFv) antibody, a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragments thereof.

136. The anti-CD3 antibody or antigen-binding fragment thereof of claim 1, wherein the anti-CD3 antibody or antigen-binding fragment thereof does not bind to CD3 at a non-acidic pH.

137. The anti-CD3 antibody or antigen-binding fragment thereof of claim 15, wherein the anti-CD3 antibody or antigen-binding fragment thereof has a binding affinity for CD3 that is 10-fold or more greater at an acidic pH than at a non-acidic pH or wherein the anti-CD3 antibody or antigen-binding fragment thereof does not bind to CD3 at a non-acidic pH.

138. The anti-CD3 antibody or antigen-binding fragment thereof of claim 67, wherein the anti-CD3 antibody or antigen-binding fragment thereof does not bind to CD3 at a non-acidic pH.

139. A method for treating cancer in a subject in need thereof comprising administering to the subject a therapeutically effective amount of the anti-CD3 antibody or antigen-binding fragment thereof of claim 1.

140. A nucleic acid encoding the anti-CD3 antibody or antigen-binding fragment thereof of claim 1.

141. A host cell comprising the nucleic acid of claim 140.

142. A method of producing an anti-CD3 antibody or antigen-binding fragment thereof comprising (a) maintaining the host cell of claim 141 in a medium to produce the anti-CD3 antibody or antigen-binding fragment thereof, and (b) isolating or purifying the anti-CD3 antibody or antigen-binding fragment thereof produced by the host cell.
